(19) United States
(12) Patent Application Publication
(10) Pub. No.: US 2023/0235085 A1
(43) Pub. Date: Jul. 27, 2023

(54) PCSK9 INHIBITORS AND METHODS OF USE THEREOF TO TREAT CHOLESTEROL-RELATED DISORDERS

(71) Applicant: Amgen Inc., Thousand Oaks, CA (US)

(72) Inventors: Andrew W. Hamer, Thousand Oaks, CA (US); Ian Matthew Bridges, Thousand Oaks, CA (US); Huei Wang, Thousand Oaks, CA (US); Andrea Ruzza, Thousand Oaks, CA (US); Christopher Kurtz, Thousand Oaks, CA (US)

(21) Appl. No.: 17/999,373

(22) PCT Filed: May 27, 2021
(86) PCT No.: PCT/US2021/034489

§ 371 Date: Nov. 18, 2022

Related U.S. Application Data
(60) Provisional application No. 63/032,451, filed on May 29, 2020.

(51) Int. Cl. C07K 16/40; A61P 3/06

(52) U.S. Cl. CPC C07K 16/40; A61P 3/06; A61K 2039/545

(57) ABSTRACT

Methods of lowering LDL cholesterol in a pediatric subject having a cholesterol-related disorder, e.g., heterozygous familial hypercholesterolemia (HeFH), by administering a PCSK9 inhibitor are provided.

### REFERENCE TO RELATED APPLICATIONS

This application claims priority to U.S. Provisional Application No. 63/032,451, filed May 29, 2020, the entirety of which is incorporated herein by reference.

### REFERENCE TO SEQUENCE LISTING

The present application is being filed along with a Sequence Listing in electronic format. The Sequence Listing is provided as a file entitled APMOL025WO.txt created on May 5, 2021, which is 149,410 bytes in size. The information in the electronic format of the Sequence Listing is incorporated herein by reference in its entirety.

### BACKGROUND

### Field

The present disclosure relates to Proprotein Convertase Subtilisin Kexin Type 9 (PCSK9) inhibitors, and to therapies targeting PCSK9 to treat cholesterol-related disorders such as heterozygous familial hypercholesterolemia (HeFH).

### SUMMARY

Provided herein is a method of lowering serum LDL cholesterol (LDL-C) in a pediatric subject, comprising: identifying a pediatric subject having heterozygous familial hypercholesterolemia (HeFH), wherein the subject has a baseline LDL-C of about 200 mg/dL or greater; and administering to the subject an anti-PCSK9 antibody at a dose from about 350 to about 500 mg, to thereby lower the subject's LDL-C. Optionally, the baseline LDL-C is from about 200 mg/dL to about 550 mg/dL. In some embodiments, the baseline LDL-C is 208 mg/dL or more. In some embodiments, the subject's LDL-C is lowered by at least 20%, at least 30%, at least 40%, about 30% to about 50%, about 20% to about 50%, about 20% to about 80%, about 30% to about 50%, or about 30% to about 80%. In some embodiments, the subject's LDL-C is lowered by at least 30%. In some embodiments, the subject's LDL-C is lowered by about 30% to about 80%. In some embodiments, the anti-PCSK9 antibody is administered every two weeks to every four weeks, every two weeks, or every four weeks. Unless explicitly stated otherwise herein, “every four weeks,” “monthly,” and “QM” as used herein shall be interchangeable. As such, administration “every four weeks” shall encompass “monthly” and “QM” administration. In some embodiments, the anti-PCSK9 antibody is administered every four weeks, and the subject's LDL-C is lowered by at least 20%. In some embodiments, the anti-PCSK9 antibody is administered every two weeks, and the subject's LDL-C is lowered by at least 30%.

Also provided herein is a method of lowering serum LDL cholesterol (LDL-C) in a pediatric subject, comprising: identifying a pediatric subject having heterozygous familial hypercholesterolemia (HeFH), wherein the subject has a baseline LDL-C of about 210 mg/dL or less, and administering to the subject a PCSK9 antibody, at a dose of about 350 to about 500 mg, to thereby lower the subject's LDL-C, wherein the subject's LDL-C is lowered by at least 40%. Optionally, the baseline LDL-C is less than 208 mg/dL. In some embodiments, the subject's LDL-C is lowered by at least 40%, at least 50%, at least 60%, about 40% to about 60%, about 40% to about 80%, about 50% to about 60%, or about 50% to about 80%. In some embodiments, the subject's LDL-C is lowered by at least 45%. In some embodiments, the anti-PCSK9 antibody is administered every two weeks to every four weeks, every two weeks, or every four weeks. In some embodiments, the anti-PCSK9 antibody is administered every four weeks, and the subject's LDL-C is lowered by at least 40%. In some embodiments, the anti-PCSK9 antibody is administered every two weeks, and the subject's LDL-C is lowered by at least 50%.

In some embodiments, the anti-PCSK9 antibody comprises: a heavy chain variable region (VH) comprising: a CDRH1, CDRH2, and a CDRH3 of a CDRH1, CDRH2, and a CDRH3, respectively, of a VH of evolocumab; and a light chain variable region (VL) comprising: a CDRL1, CDRL2, and a CDRL3 of a CDRL1, CDRL2, and a CDRL3, respectively, of a VL of evolocumab. In some embodiments, the anti-PCSK9 antibody comprises: a heavy chain variable region (VH) comprising an amino acid sequence at least 90% identical to the VH of evolocumab; and a light chain variable region (VL) comprising an amino acid sequence at least 90% identical to the VL of evolocumab. In some embodiments, the anti-PCSK9 antibody comprises: a heavy chain variable region (VH) comprising an amino acid sequence at least 95% identical to the VH of evolocumab; and a light chain variable region (VL) comprising an amino acid sequence at least 95% identical to the VL of evolocumab. In some embodiments, the anti-PCSK9 antibody comprises: a heavy chain variable region (VH) comprising: a CDRH1, CDRH2, and a CDRH3 of a CDRH1, CDRH2, and a CDRH3, respectively, of a VH of evolocumab; and an amino acid sequence at least 90% identical to the VH of evolocumab; and a light chain variable region (VL) comprising: a CDRL1, CDRL2, and a CDRL3 of a CDRL1, CDRL2, and a CDRL3, respectively, of a VL of evolocumab; and an amino acid sequence at least 90% identical to the VL of evolocumab. Optionally, the anti-PCSK9 antibody is evolocumab.

In some embodiments, the dose is about 420 mg. In some embodiments, the dose is about 490 mg.

In some embodiments, the subject's LDL-C is lowered by at least week 20 of administration of the PCSK9 antibody.

Provided herein is a method of lowering serum LDL cholesterol (LDL-C) in a pediatric subject, comprising: identifying a pediatric subject having heterozygous familial hypercholesterolemia (HeFH), wherein the subject has a baseline LDL-C at or above an upper quartile of baseline LDL-C values among a pediatric HeFH patient cohort; and administering to the subject an enhanced dosage regimen of a PCSK9 inhibitor, wherein the enhanced dosage regimen comprises an amount and/or dosing frequency that is each independently about 20% to about 500% greater than an average amount and/or average dosing frequency of the PCSK9 inhibitor for pediatric HeFH patients having a baseline LDL-C value that is less than the upper quartile, whereby the subject's LDL-C is lowered. Optionally, the amount of the PCSK9 inhibitor is about 5% to about 100% greater than the average amount. In some embodiments, the dosing frequency of the PCSK9 inhibitor is about 15% to about 40% greater than the average dosing frequency. In some embodiments, the average dosing frequency is a dosing frequency of the PCSK9 inhibitor for pediatric HeFH patients having a baseline LDL-C value that is less than a median of baseline LDL-C values among the cohort. In some embodiments, the average amount is an amount of the PCSK9 inhibitor for pediatric HeFH patients having a baseline LDL-C value that is less than a median of baseline LDL-C values among the cohort. In some embodiments, the subject's LDL-C is lowered by at least 30%. In some embodiments, the subject's LDL-C is lowered by from about 30% to about 80%. In some embodiments, the reduction in the subject's LDL-C is at least 70% of the average reduction in LDL-C achieved in pediatric HeFH patients having a baseline LDL-C value that is less than the upper quartile and receiving the PCSK9 inhibitor at the average frequency of administration. In some embodiments, the subject's LDL-C is lowered by at least week 20 of administration of the PCSK9 inhibitor.

Also provided is a method of treating or preventing heterozygous familial hypercholesterolemia (HeFH) or symptoms thereof, comprising: identifying a pediatric subject in need of treatment or prevention of HeFH or symptoms thereof, wherein the subject has a baseline LDL-C at or above an upper quartile of baseline LDL-C values among a pediatric HeFH patient cohort; and administering to the subject an enhanced dosage regimen of a PCSK9 inhibitor, to thereby treat or prevent HeFH or symptoms thereof, wherein the enhanced dosage regimen comprises administering the PCSK9 inhibitor at a mean dose that is about 20% to about 500% greater than a reference mean dose of the PCSK9 inhibitor for treating or preventing HeFH or symptoms thereof in pediatric HeFH patients having a baseline LDL-C value that is less than the upper quartile. Optionally, the reference mean dose is a dose of the PCSK9 inhibitor for pediatric HeFH patients having a baseline LDL-C value that is less than a median of baseline LDL-C values among the cohort. In some embodiments, the enhanced dosage regimen comprises an increase in a dosing frequency and/or an amount of the PCSK9 inhibitor administered to the subject. In some embodiments, the upper quartile is in a range of about 190 mg/dL to about 220 mg/dL. In some embodiments, the upper quartile is about 200 mg/dL. In some embodiments, the subject's baseline LDL-C is about 200 mg/dL or greater. In some embodiments, the baseline LDL-C is from about 200 mg/dL to about 550 mg/dL. In some embodiments, the baseline LDL-C is 208 mg/dL or greater. In some embodiments, the PCSK9 inhibitor is approved by a government regulatory agency for lowering serum LDL cholesterol levels in a human patient. In some embodiments, the average dosing frequency is a dosing frequency of the PCSK9 inhibitor for pediatric HeFH patients having a baseline LDL-C value that is less than a median of baseline LDL-C values among the cohort. In some embodiments, the PCSK9 inhibitor is an antibody, small-molecule inhibitor, or an inhibitory nucleic acid. Optionally, the PCSK9 inhibitor is an anti-PCSK9 antibody, a siRNA or shRNA. Optionally, the PCSK9 inhibitor comprises one or more of evolocumab, alirocumab, bococizumab, 1D05-IgG2, RG-7652, LGT209, REGN728, LY3015014, 1B20, inclisiran, ISIS 394814, ALN-PCS02, SX-PCK9, and BMS-962476. In some embodiments, the average dosing frequency is in a range from about once every 2 weeks to about once every 12 weeks. In some embodiments, the method further comprises determining quartiles of the baseline LDL-C values of the cohort. In some embodiments, the cohort comprises at least 25 pediatric HeFH patients. In some embodiments, the baseline LDL-C values among the cohort is at least 130 mg/dL.

In some embodiments, the method further comprises measuring the baseline LDL-C of the subject. In some embodiments, the identifying comprises diagnosing and/or genotyping the subject for HeFH. In some embodiments, the identifying comprises diagnosing and/or genotyping the patient for compound HeFH.

Also provided herein is a method of lowering serum LDL-cholesterol (LDL-C) in a pediatric subject, the method comprising: administering to a pediatric subject an enhanced dosage regimen of a PCSK9 inhibitor, wherein the subject has heterozygous familial hypercholesterolemia (HeFH) or symptoms thereof, wherein the enhanced dosage regimen of the PCSK9 inhibitor comprises an amount of the PCSK9 inhibitor that is about 20% to about 500% greater than a standard-of-care average amount for adults having HeFH, and/or a dosing frequency of the PCSK9 inhibitor that is about 20% to about 500% greater than a standard-of-care average frequency for adults having HeFH, whereby the subject's LDL-C is lowered. Optionally, the enhanced dosage regimen lowers LDL-C in the subject by at least 30%. In some embodiments, the enhanced dosage regimen lowers LDL-C in the subject by 30%-80%. In some embodiments, the amount of the PCSK9 inhibitor is increased by about 5% to about 100% than the standard-of-care amount. In some embodiments, the dosing frequency of the PCSK9 inhibitor is increased by about 15% to about 400% than the standard-of-care dosing frequency. In some embodiments, the enhanced dosage regimen is continued until a therapeutically acceptable end point for HeFH is achieved. In some embodiments, the PCSK9 inhibitor is approved by government regulatory agency for lowering LDL-C in a human patient. In some embodiments, the standard-of-care dosing frequency is between once every 2 weeks to once every 12 weeks. In some embodiments, the PCSK9 inhibitor is an anti-PCSK9 antibody. Optionally, the anti-PCSK9 antibody comprises: a heavy chain variable region (VH) comprising: a CDRH1, CDRH2, and a CDRH3 of a CDRH1, CDRH2, and a CDRH3, respectively, of a VH of evolocumab; and an amino acid sequence at least 90% identical to the VH of evolocumab; and a light chain variable region (VL) comprising: a CDRL1, CDRL2, and a CDRL3 of a CDRL1, CDRL2, and a CDRL3, respectively, of a VL of evolocumab; and an amino acid sequence at least 90% identical to the VL of evolocumab. In some embodiments, the anti-PCSK9 antibody is evolocumab.

In some embodiments, the standard-of-care amount is between 400 and 500 mg/dose. In some embodiments, the standard-of-care amount and/or frequency is about 420 mg/month.

In some embodiments, the subject's LDL-C is lowered by at least week 20 of administration of the PCSK9 inhibitor.

Provided herein is a method of treating or preventing heterozygous familial hypercholesterolemia (HeFH) or symptoms thereof, comprising: identifying a pediatric subject in need of treatment or prevention of HeFH or symptoms thereof; and administering to the subject an enhanced dosage regimen of a PCSK9 inhibitor, to thereby treat or prevent HeFH or symptoms thereof, wherein the enhanced dosage regimen comprises administering the PCSK9 inhibitor at an mean dose that is about 20% to about 500% greater than a standard-of-care mean dose of the PCSK9 inhibitor to treat or prevent HeFH or symptoms thereof in an adult patient. Optionally, the enhanced dosage regimen comprises a higher dosing frequency of the PCSK9 inhibitor than a standard-of-care dosing frequency. In some embodiments, the enhanced dosage regimen comprises a higher amount of the PCSK9 inhibitor than a standard-of-care amount. In some embodiments, the PCSK9 inhibitor is an antibody, small-molecule inhibitor, or an inhibitory nucleic acid. Optionally, the PCSK9 inhibitor is an anti-PCSK9 antibody. Optionally, the PCSK9 inhibitor is a siRNA or shRNA. Optionally, the PCSK9 inhibitor comprises one or more of evolocumab, alirocumab, bococizumab, 1D05-IgG2, RG-7652, LGT209, inclisiran, ISIS 394814, SX-PCK9, and BMS-962476.

In some embodiments, the method further comprises administering one or more other LDL cholesterol-lowering therapy to the subject. Optionally, the other LDL cholesterol-lowering therapy comprises a statin, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IB AT inhibitor, a squalene synthase inhibitor, or a monocyte chemoattractant protein (MCP)-I inhibitor.

Also provided is a method of lowering serum LDL cholesterol (LDL-C) in a pediatric subject, comprising: administering to a pediatric subject having HeFH, wherein the subject has a baseline LDL-C of about 200 mg/dL or greater: a PCSK9 antibody at a dosing frequency of about once a month, and at an amount from about 400 mg to about 450 mg; at least one statin; and at least one other LDL cholesterol-lowering therapy that is different from the PCSK9 antibody and the at least one statin, to thereby lower the subject's LDL-C by at least 30%. Optionally, the anti-PCSK9 antibody comprises: a heavy chain variable region (VH) comprising: a CDRH1, CDRH2, and a CDRH3 of a CDRH1, CDRH2, and a CDRH3, respectively, of a VH of evolocumab; and an amino acid sequence at least 90°/% identical to the VH of evolocumab; and a light chain variable region (VL) comprising: a CDRL1, CDRL2, and a CDRL3 of a CDRL1, CDRL2, and a CDRL3, respectively, of a VL of evolocumab; and an amino acid sequence at least 90% identical to the VL of evolocumab. Optionally, the anti-PCSK9 antibody is evolocumab. In some embodiments, the amount is about 420 mg.

Also provided herein is a method of treating or preventing heterozygous familial hypercholesterolemia (HeFH) or symptoms thereof, comprising: administering to a pediatric subject having HeFH and a baseline serum LDL cholesterol (LDL-C) at or above an upper quartile of baseline LDL-C values among a pediatric HeFH patient cohort: a PCSK9 inhibitor; at least one statin; and at least one other LDL cholesterol-lowering therapy that is different from the PCSK9 inhibitor and the at least one statin, to thereby treat or prevent HeFH or symptoms thereof, wherein the PCSK9 inhibitor is administered according to a dosage regimen of the PCSK9 inhibitor for pediatric HeFH patients having a baseline LDL-C value that is less than the upper quartile. Optionally, the upper quartile is in a range of about 190 mg/dL to about 220 mg/dL. In some embodiments, the baseline LDL-C is about 200 mg/dL or greater. In some embodiments, the PCSK9 inhibitor is administered according to a dosage regimen of the PCSK9 inhibitor for pediatric HeFH patients having a baseline LDL-C value that is less than a median of baseline LDL-C values among the cohort.

Provided herein is a method of treating or preventing heterozygous familial hypercholesterolemia (HeFH) or symptoms thereof, comprising: administering to a pediatric subject having HeFH: a PCSK9 inhibitor, wherein the PCSK9 inhibitor is administered according to a standard-of-care dosage regimen to treat or prevent HeFH or symptoms thereof in an adult patient; at least one statin; and at least one other LDL cholesterol-lowering therapy that is different from the PCSK9 inhibitor and the at least one statin, to thereby treat or prevent HeFH or symptoms thereof. Optionally, the at least one other LDL cholesterol-lowering therapy is administered according to an enhanced dosage regimen comprising an mean dose of the at least one other LDL cholesterol-lowering therapy that is about 20% to about 500% greater than a standard-of-care mean dose of the at least one other LDL cholesterol-lowering therapy to treat or prevent HeFH or symptoms thereof in a pediatric patient. Optionally, the enhanced dosage regiment comprises an increase in a dosing frequency and/or an increase in an amount of the PCSK9 inhibitor.

In some embodiments, the PCSK9 inhibitor is an antibody, small-molecule inhibitor, or an inhibitory nucleic acid. Optionally, the PCSK9 inhibitor comprises one or more of evolocumab, alirocumab, bococizumab, 1D05-IgG2, RG-7652, LGT209, REGN728, LY3015014, 1B20, inclisiran, ISIS 394814, SX-PCK9, and BMS-962476. In some embodiments, the at least one other LDL cholesterol-lowering therapy comprises a second PCSK9 inhibitor. Optionally, the second PCSK9 inhibitor is a small-molecule inhibitor, or an inhibitory nucleic acid. Optionally, the second PCSK9 inhibitor comprises one or more of evolocumab, alirocumab, bococizumab, 1D05-IgG2, RG-7652, LGT209, REGN728, LY3015014, 1B20, inclisiran, ISIS 394814, SX-PCK9, and BMS-962476.

In some embodiments, the at least one other LDL cholesterol-lowering therapy comprises a statin, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IB AT inhibitor, a squalene synthase inhibitor, or a monocyte chemoattractant protein (MCP)-I inhibitor.

In some embodiments, the age of the subject is 17 years old or younger. In some embodiments, the age of the subject is between 10 and 17 years old. In some embodiments, the subject has compound HeFH. In some embodiments, the subject is receiving at least one other LDL cholesterol-lowering therapy. In some embodiments, the PCSK9 inhibitor or anti-PCSK9 antibody is administered subcutaneously or intravenously.

Provided herein is a kit for treating a pediatric subject in need of treating or preventing heterozygous familial hypercholesterolemia (HeFH) or symptoms thereof, comprising: a dosage form comprising a PCSK9 inhibitor in an amount sufficient to administer the PCSK9 inhibitor to a pediatric subject having HeFH an enhanced dosage regimen comprising administering to the subject the PCSK9 inhibitor at a dosing frequency that is at least 2 fold greater than an average dosing frequency of the PCSK9 inhibitor for pediatric HeFH patients having a baseline LDL-C value that is less than the upper quartile. Optionally, the average dosing frequency is a dosing frequency of the PCSK9 inhibitor for pediatric HeFH patients having a baseline LDL-C value that is less than a median of baseline LDL-C values among the cohort.

Also provided is a kit for treating a pediatric subject in need of treating or preventing heterozygous familial hypercholesterolemia (HeFH) or symptoms thereof, comprising: a dosage form comprising a PCSK9 inhibitor in an amount sufficient to administer the PCSK9 inhibitor to a pediatric subject an enhanced dosage regimen comprising administering to the subject the PCSK9 inhibitor at a dosage that is about 20% to about 500% greater than a standard-of-care dosage of the PCSK9 inhibitor to treat or prevent the cholesterol-related disorder in an adult HeFH patient.

Also provided herein is a method of lowering serum LDL cholesterol (LDL-C), comprising: administering to a subject a PCSK9 inhibitor, wherein the subject has heterozygous familial hypercholesterolemia, wherein the subject is a pediatric subject, wherein the PCSK9 inhibitor is administered in an amount that is at least as effective as 420 mg of evolocumab, wherein the PCSK9 inhibitor is administered at a frequency of every two weeks or more, whereby the subject's LDL-C is reduced by more than 30%.

Provided herein is a method of lowering serum LDL cholesterol (LDL-C) in a subject, comprising: identifying a pediatric subject having heterozygous familial hypercholesterolemia (HeFH), wherein the subject has a baseline LDL-C above an upper quartile of baseline LDL-C values among a pediatric HeFH patient cohort; and administering to the subject an enhanced dosage regimen of a PCSK9 inhibitor, wherein the enhanced dosage regimen comprises a dosing frequency and/or an amount that is from 20% to 500% greater than an average dosing frequency and/or average amount in a government regulatory agency-approved label for the PCSK9 inhibitor, whereby the subject's LDL-C is lowered by at least 30%. Optionally, the enhanced dosage regimen comprises a dosing frequency that is at least 2 fold greater than the average dosing frequency.

In some embodiments, the PCSK9 inhibitor comprises one or more of evolocumab, alirocumab, bococizumab, 1D05-IgG2, RG-7652, LGT209, REGN728, LY3015014, 1B20, inclisiran, ISIS 394814, ALN-PCS02, SX-PCK9, and BMS-962476.

### BRIEF DESCRIPTION OF THE DRAWINGS

FIG. 1 shows a schematic diagram of a study design for evolocumab for low density lipoprotein-cholesterol (LDL-C) reduction in pediatric subjects with heterozygous familial hypercholesterolemia (HeFH).

FIGS. 2A and 2B show schematic diagrams of methods of lowering serum LDL cholesterol (LDL-C) in a pediatric subject, according to some embodiments of the present disclosure.

FIG. 3 shows a schematic diagram of a method of lowering serum LDL cholesterol (LDL-C) in a pediatric subject, according to some embodiments of the present disclosure.

FIG. 4 shows a schematic diagram of a method of treating or preventing heterozygous familial hypercholesterolemia (HeFH) or symptoms thereof, according to some embodiments of the present disclosure.

FIG. 5 shows a schematic diagram of a method of lowering serum LDL cholesterol (LDL-C) in a pediatric subject, according to some embodiments of the present disclosure.

FIG. 6 shows a schematic diagram of a method of treating or preventing heterozygous familial hypercholesterolemia (HeFH) or symptoms thereof, according to some embodiments of the present disclosure.

FIG. 7 shows a schematic diagram of a method of lowering serum LDL cholesterol (LDL-C) in a pediatric subject, according to some embodiments of the present disclosure.

FIG. 8 shows a schematic diagram of a method of treating or preventing heterozygous familial hypercholesterolemia (HeFH) or symptoms thereof, according to some embodiments of the present disclosure.

FIG. 9 shows a schematic diagram of a method of treating or preventing heterozygous familial hypercholesterolemia (HeFH) or symptoms thereof, according to some embodiments of the present disclosure.

FIGS. 10A and 10B show amino acid sequences of the mature form of PCSK9 with the pro-domain underlined.

FIG. 11 shows a nucleic acid sequences of human PCSK9 with the sequence encoding the signal sequence in bold.

FIG. 12 shows an amino acid of human PCSK9 with the pro-domain underlined and the signal sequence in bold.

FIG. 13 shows the amino acid and nucleic acid sequences of human PCSK9 with the pro-domain underlined and the signal sequence in bold.

FIG. 14 shows some sequence aspects of some embodiments of PCSK9 inhibitors.

FIGS. 15A and 15B show some sequence aspects of some embodiments of PCSK9 inhibitors. The highlighted regions denote the variable regions.

FIG. 16 shows some sequence aspects of some embodiments of PCSK9 inhibitors.

FIG. 17 shows some sequence aspects of some embodiments of PCSK9 inhibitors.

FIG. 18 shows some sequence aspects of some embodiments of PCSK9 inhibitors.

FIG. 19 shows some sequence aspects of some embodiments of PCSK9 inhibitors.

FIG. 20 shows some constant domain sequence aspects of some embodiments of PCSK9 inhibitors.

### DETAILED DESCRIPTION

Methods of treating a subject, e.g., a pediatric subject, having a cholesterol-related disorder, e.g., familial hypercholesterolemia (FH), such as heterozygous FH (HeFH), using a PCSK9 inhibitor is provided. As disclosed herein, the efficacy of a PCSK9 inhibitor for the treatment or prevention of a cholesterol-related disorder, e.g., HeFH, can depend on a variety of factors, such as age of the subject, severity of the subject's disorder as measured by baseline serum LDL cholesterol (LDL-C) levels, and/or the subject's genotype. For example, HeFH can comprise compound heterozygous FH, which can be relatively severe compared to heterozygotes that comprise a wild-type allele of the subject gene. Among a cohort of pediatric HeFH patients, each patient's baseline LDL-C can vary. Without being bound by theory, in line with the results presented herein, the response to a PCSK9 inhibitor therapy, e.g., an anti-PCSK9 antibody therapy, may be blunted when the pediatric HeFH subject receiving treatment has a more severe form of the cholesterol-related disorder reflected, for example, in the subject's baseline LDL-C level being at or above an upper quartile of baseline LDL-C levels among a cohort of pediatric HeFH patients. As disclosed herein, the reduction in LDL-C in response to a PCSK9 inhibitor therapy in a pediatric HeFH subject having a baseline LDL-C of about 200 mg/dL or greater, e.g., a baseline LDL-C of 208 mg/dL or greater, can be attenuated compared to the reduction achieved by the same PCSK9 inhibitor therapy in a pediatric HeFH patient having a baseline LDL-C lower than about 200 mg/dL, e.g., a baseline LDL-C lower than 208 mg/dL. A pediatric HeFH subject having severe HeFH, e.g., having a baseline LDL-C level at or above the upper quartile, can benefit from an enhanced dosage regimen to compensate for the blunted response to the PCSK9 inhibitor therapy. In some embodiments, the enhanced dosage regimen includes an increased frequency and/or dosage amount of administration compared to the frequency and/or dosage amount of administration of a dosage regimen for pediatric HeFH patients having a baseline LDL-C that is less than the upper quartile.

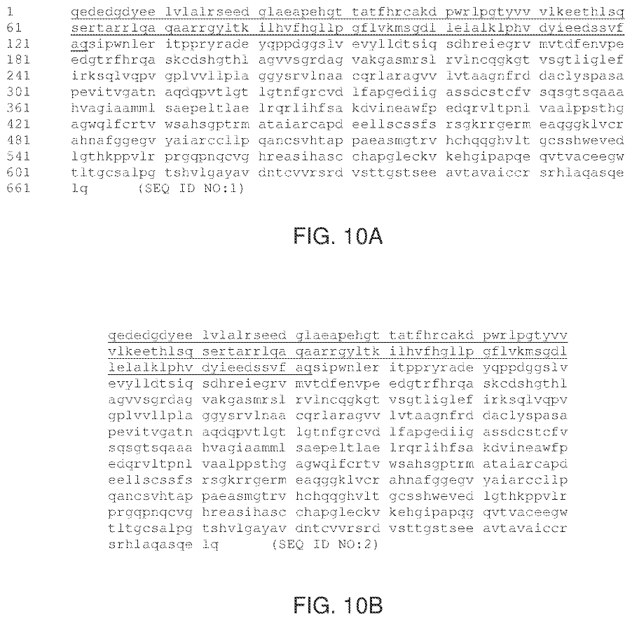

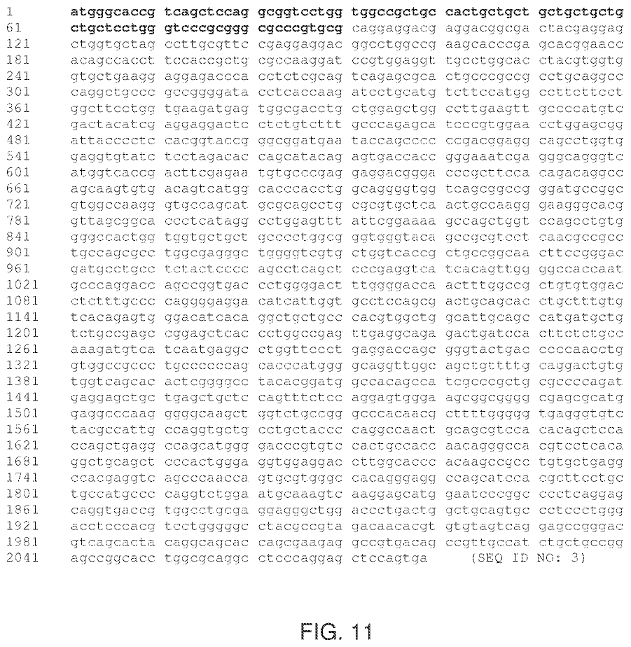

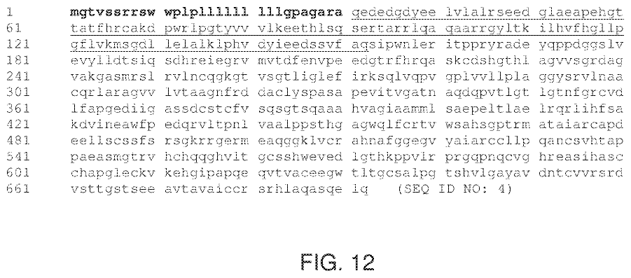

The term “proprotein convertase subtilisin kexin type 9” or “PCSK9” refers to a polypeptide as set forth in SEQ ID NO: 1, 2, 4 and/or 6 in FIGS. 10A, 10B, 12, and 13. “PCSK9” has also been referred to as FH3, NARC1, HCHOLA3, proprotein convertase subtilisin/kexin type 9, and neural apoptosis regulated convertase 1. The PCSK9 gene encodes a proprotein convertase protein that belongs to the proteinase K subfamily of the secretory subtilase family. The term “PCSK9” denotes both the proprotein and the product generated following autocatalysis of the proprotein. When only the autocatalyzed product is being referred to (such as for an antibody that selectively binds to the cleaved PCSK9), the protein can be referred to as the “mature,” “cleaved”, “processed” or “active” PCSK9. When only the inactive form is being referred to, the protein can be referred to as the “inactive”, “pro-form”, or “unprocessed” form of PCSK9.

“PCSK9 inhibitor” denotes a molecule or therapy that inhibits PCSK9 activity to thereby lower LDL-C (and/or other lipids, such as non-HDL-C, ApoB, Lp(a), etc.) levels. This can include neutralizing antibodies to PCSK9 and anti-sense molecules to PCSK9, for example. A PCSK9 inhibitor therapy denotes a method that uses a PCSK9 inhibitor agent.

“Baseline” as used herein with reference to serum LDL cholesterol (LDL-C) refers to the level of serum LDL-C in a subject who has not been administered a PCSK9 inhibitor for treatment of a cholesterol-related disorder, e.g., HeFH, or for prevention of symptoms thereof. Generally, the baseline is a fasting LDL-C. In some embodiments, a subject is taking a (non-PCSK9 inhibitor) LDL-C-lowering therapy, such as a statin, when the baseline LDL-C is established.

“Median” as used herein in reference to LDL-C levels in a patient cohort, is the LDL-C value separating the higher half from the lower half of all the LDL-C levels in the cohort. Half the LDL-C values in the cohort are below the median, and half are above. An “upper quartile” is the LDL-C value separating the top 25% from the lowest 75% of all the LDL-C levels in the cohort. A “lower quartile” is the LDL-C value separating the bottom 25% from the highest 75% of all the LDL-C levels in the cohort.

“Standard-or-care” as used herein has its customary and plain meaning as understood by one of ordinary skill in the art, in view of the present disclosure. In some embodiments, the standard-of-care includes guidelines for a course of action, e.g., administration of a therapeutic agent to treat a disorder, that are generally accepted by practitioners to be safe and effective for achieving the intended purpose. In some embodiments, the standard-of-care includes a government-approved guideline for using a therapeutic agent to treat a patient. In some embodiments, the standard-of-care includes a guideline accepted by a government regulatory agency (e.g., the U.S. Food and Drug Administration (FDA) or the European Medicines Agency (EMA)). In some embodiments, the standard-of-care for a therapeutic agent applies to a specific patient population, e.g., adult patients.

“Government regulatory agency” as used herein refers to a national, international or local governmental organization that is tasked with approving therapies for treating a disease or disorder in patients, e.g., human patients. Suitable government regulatory agencies include, without limitation, the FDA, European Medicines Agency (EMA), Pharmaceuticals and Medical Devices Agency (Japan), National Medical Products Administration (China), Health Canada, Medicines and Healthcare Products Regulatory Agency (UK), Central Drug Standard Control Organization (India), and Therapeutic Goods Administration (Australia).

An “antibody” refers to an immunoglobulin of any isotype, and includes, for instance, chimeric, humanized, fully human, and monoclonal antibodies. An “antibody” as such is a subgenus of an antigen binding protein. For example, human antibodies can be of any isotype, including IgG (including IgG1, IgG2, IgG3 and IgG4 subtypes), IgA (including IgA1 and IgA2 subtypes), IgM and IgE. A human IgG antibody generally will comprise two full-length heavy chains and two full-length light chains. Antibodies may be derived solely from a single source, or may be “chimeric,” that is, different portions of the antibody may be derived from two or more different antibodies from the same or different species. For the purposes of illustration, PCSK9 protein (e.g., having the amino acid sequence of any one of SEQ ID NOs: 1, 2, 4, or 6) or a fragment thereof is an example of an antigen for an anti-PCSK9 antibody. A canonical immunoglobulin is a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

By way of example, antibodies having 1, 2, 3, 4, or 5 amino acid residue substitutions, insertions or deletions at the N-terminus and/or C-terminus of the heavy and/or light chains are included in the definition provided that the antibodies retain the same or similar binding and/or function as the antibodies comprising two full length heavy chains and two full length light chains.

Antigen binding proteins that bind to PCSK9 are also described herein. An antigen binding protein may comprise, consist essentially of, or consist of a fragment of an anti-PCSK9 antibody. In some embodiments, an antigen binding protein to PCSK9 may be substituted for an anti-PCSK9 antibody as described herein. Antigen binding proteins can include antibody fragments (e.g., an antigen binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)2 fragment, a Fv fragment, a Fd fragment, and a complementarity determining region (CDR) fragment. These molecules can be derived from any mammalian source, such as human, mouse, rat, rabbit, or pig, dog, or camelid. Antibody fragments may compete for binding of a target antigen with an intact antibody and the fragments may be produced by the modification of intact antibodies (e.g. enzymatic or chemical cleavage) or synthesized de novo using recombinant DNA technologies or peptide synthesis. The antibody can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129 (2003); Roque et al., Biotechnol. Prog. 20:639-654 (2004). In addition, peptide antibody mimetics (“PAMs”) can be used, as well as scaffolds based on antibody mimetics utilizing fibronectin components as a scaffold. In some embodiments, an antigen binding fragment of an antibody comprises at least one CDR from an antibody that binds to the antigen, and in some embodiments comprises the heavy chain CDR3 from an antibody that binds to the antigen. In some embodiments, the antigen binding fragment comprises all three CDRs from the heavy chain of an antibody that binds to the antigen or from the light chain of an antibody that binds to the antigen. In some embodiments, the antigen binding fragment comprises all six CDRs from an antibody that binds to the antigen (three from the heavy chain and three from the light chain). The antigen binding fragment in certain embodiments is an antibody fragment.

An antigen binding protein can also include a protein comprising one or more antibody fragments incorporated into a single polypeptide chain or into multiple polypeptide chains. For instance, antigen binding protein can include, but are not limited to, a diabody (see, e.g., EP 404,097; WO 93/11161; and Hollinger et al., Proc. Natl. Acad. Sci. USA, Vol. 90:6444-6448, 1993); an intrabody; a domain antibody (single VL or VH domain or two or more VH domains joined by a peptide linker; see Ward et al., Nature, Vol. 341:544-546, 1989); a maxibody (2 scFvs fused to Fc region, see Fredericks et al., Protein Engineering, Design & Selection, Vol. 17:95-106, 2004 and Powers et al., Journal of Immunological Methods, Vol. 251:123-135, 2001); a triabody; a tetrabody; a minibody (scFv fused to CH3 domain; see Olafsen et al., Protein Eng Des Sel., Vol. 17:315-23, 2004); a peptibody (one or more peptides attached to an Fc region, see WO 00/24782); a linear antibody (a pair of tandem Fd segments (VH-CH1-VH-CH1) which, together with complementary light chain polypeptides, form a pair of antigen binding regions, see Zapata et al., Protein Eng., Vol. 8:1057-1062, 1995); a small modular immunopharmaceutical (see U.S. Patent Publication No. 20030133939); and immunoglobulin fusion proteins (e.g. IgG-scFv, IgG-Fab, 2scFv-IgG, 4scFv-IgG, VH-IgG, IgG-VH, and Fab-scFv-Fc).

Within light and heavy chains, the variable (V) and constant regions (C) are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

Human light chains are classified as kappa and lambda light chains. The term “light chain” refers to a polypeptide comprising, from amino terminus to carboxyl terminus, a single immunoglobulin light chain variable region (VL) and a single immunoglobulin light chain constant domain (CL). Heavy chains are classified as mu (μ), delta (Δ), gamma (γ), alpha (α), and epsilon (ε), and define the antibody's isotype as IgM, IgD, IgG, IgA, and IgE, respectively. The term “heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). The IgG-class is further divided into subclasses, namely, IgG1, IgG2, IgG3, and IgG4. The IgA-class is further divided into subclasses, namely IgA1 and IgA2. The IgM has subclasses including, but not limited to, IgM1 and IgM2. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the antibody heavy chains.

A “polyclonal antibody” refers to a population of antibodies that are typically widely varied in composition and binding specificity. A “monoclonal antibody” (“mAb”) as used herein refers to one or more of a population of antibodies having identical sequences. Monoclonal antibodies bind to the antigen at a particular epitope on the antigen.

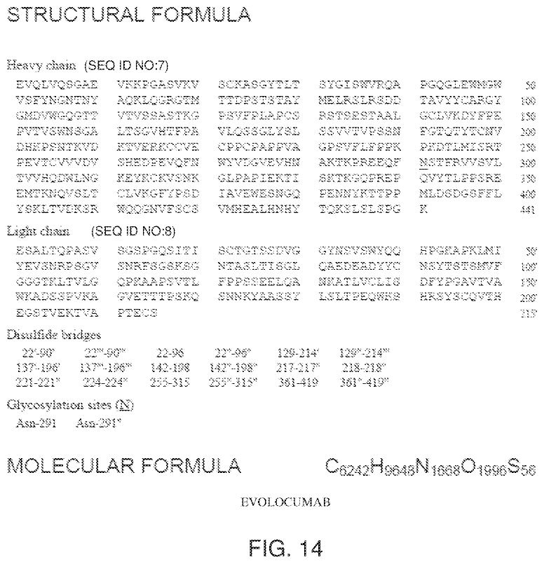

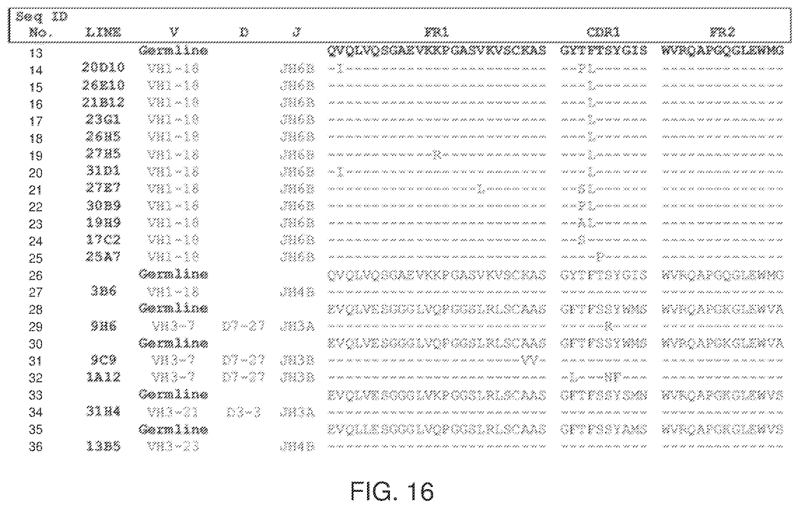

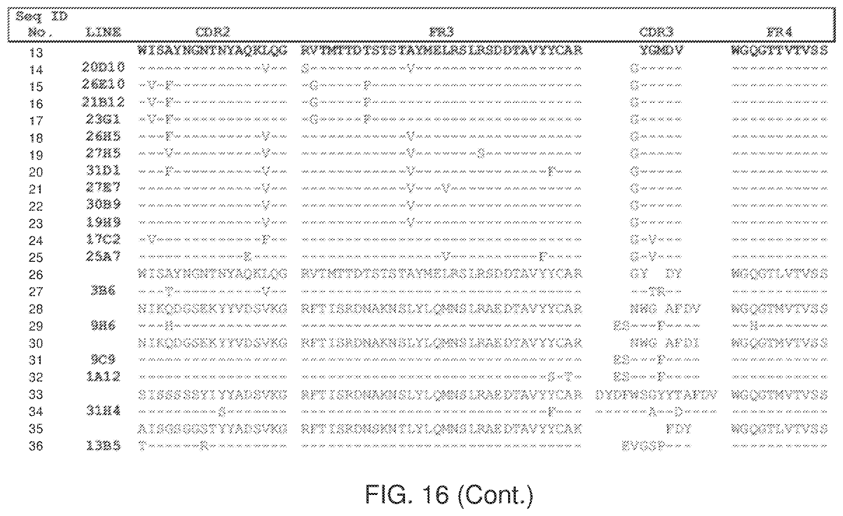

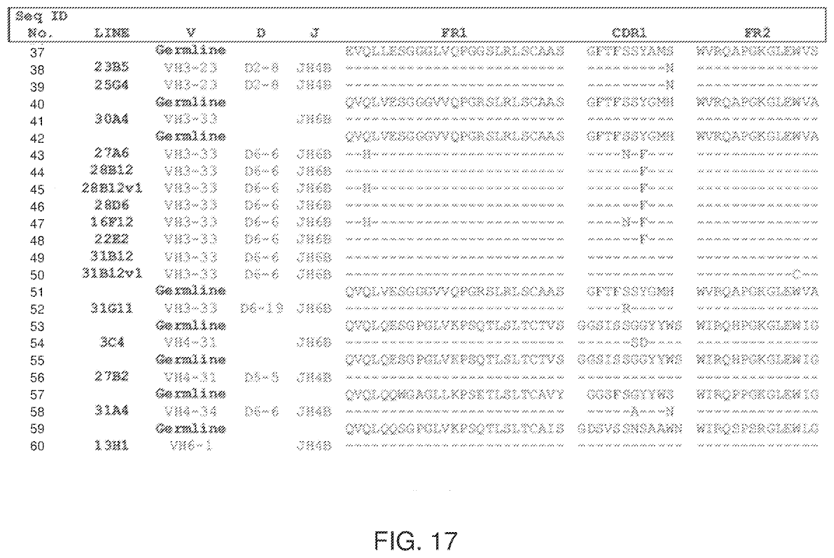

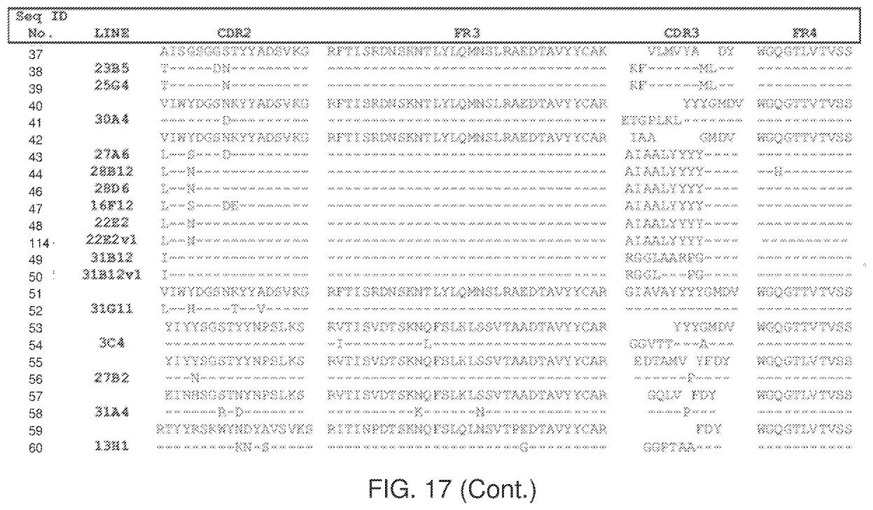

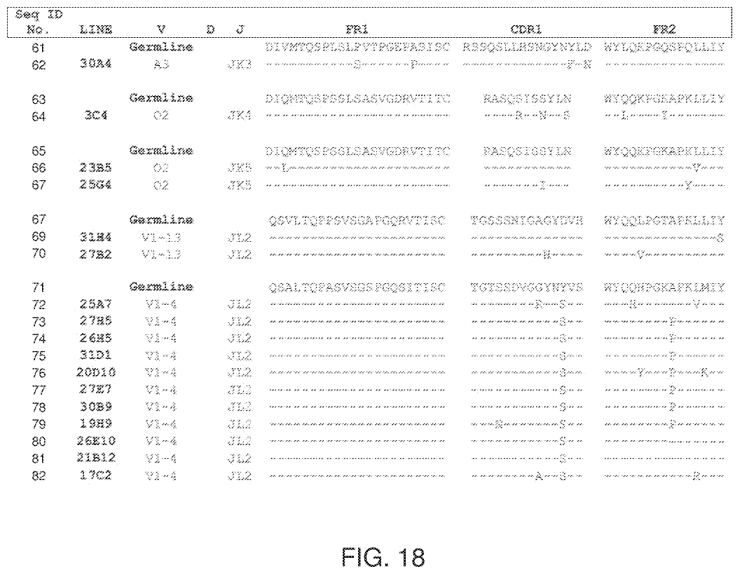

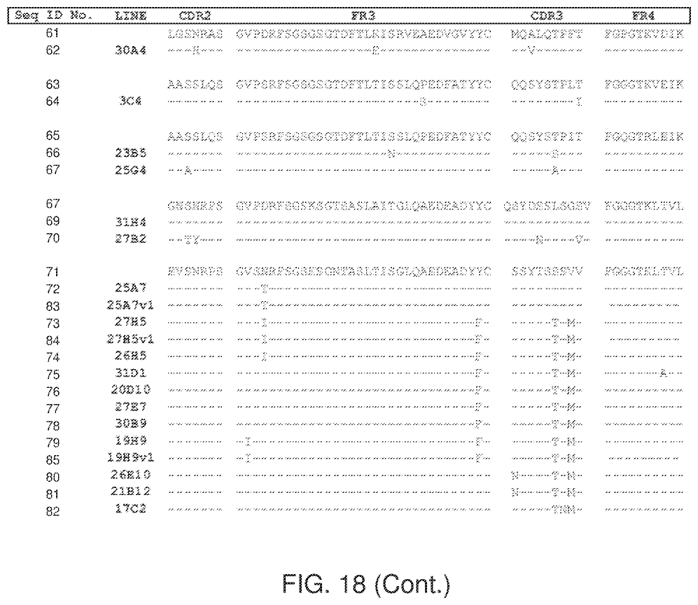

In some embodiments, an anti-PCSK9 antibody comprises at least one CDR set forth in FIGS. 14, 15A, 15B, 16, 17, 18, and/or 19. In some embodiments, an anti-PCSK9 antibody comprises the HCDR1, HCDR2, HCDR3, LCDR1, LCDR2, and LCDR3 of an antibody as set forth in FIGS. 14, 15A, 15B, 16, 17, 18, and/or 19. In some embodiments, the anti-PCSK9 antibody comprises: a heavy chain variable region (VH) comprising an amino acid sequence at least 90/o identical to the VH of evolocumab; and a light chain variable region (VL) comprising an amino acid sequence at least 90% identical to the VL as set forth in FIGS. 14, 15A, 15B, 16, 17, 18, and/or 19. In some embodiments, the anti-PCSK9 antibody comprises: a heavy chain variable region (VH) comprising an amino acid sequence at least 95% identical to the VH of evolocumab; and a light chain variable region (VL) comprising an amino acid sequence at least 95% identical to the VL as set forth in FIGS. 14, 15A, 15B, 16, 17, 18, and/or 19.

The term “CDR” refers to the complementarity determining region (also termed “minimal recognition units” or “hypervariable region”) within antibody variable sequences. The CDRs permit the antibody to specifically bind to a particular antigen of interest. There are three heavy chain variable region CDRs (CDRH1, CDRH2 and CDRH3) and three light chain variable region CDRs (CDRL1, CDRL2 and CDRL3). The CDRs in each of the two chains typically are aligned by the framework regions to form a structure that binds specifically to a specific epitope or domain on the target protein. From N-terminus to C-terminus, naturally-occurring light and heavy chain variable regions both typically conform to the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, Md.), or Chothia & Lesk, 1987, J. Mol. Biol. 196:901-917; Chothia et al., 1989, Nature 342:878-883. Complementarity determining regions (CDRs) and framework regions (FR) of a given antibody may be identified using this system. Other numbering systems for the amino acids in immunoglobulin chains include IMGT® (the international ImMunoGeneTics information system; Lefranc et al, Dev. Comp. Immunol. 29:185-203; 2005) and AHo (Honegger and Pluckthun, J. Mol. Biol. 309(3):657-670; 2001). One or more CDRs may be incorporated into a molecule either covalently or noncovalently to make it an antibody.

In some embodiments, an antigen binding protein of the present disclosure may incorporate the CDR(s) as part of a larger polypeptide chain, may covalently link the CDR(s) to another polypeptide chain, or may incorporate the CDR(s) noncovalently. The antigen binding proteins may comprise at least one of the CDRs described herein incorporated into a biocompatible framework structure. In one example, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions or substitutions of amino acids, relative to a naturally occurring polypeptide or fold. These scaffolds can be derived from a polypeptide of any species (or of more than one species), such as a human, other mammal, other vertebrate, invertebrate, plant, bacteria or virus.

Typically the biocompatible framework structures are based on protein scaffolds or skeletons other than immunoglobulin domains. For example, those based on fibronectin, ankyrin, lipocalin, neocarzinostain, cytochrome b, CPI zinc finger, PST1, coiled coil, LACI-D1, Z domain and tendamistat domains may be used (See e.g., Nygren and Uhlen, 1997, Current Opinion in Structural Biology, 7, 463-469).

In some embodiments, an antigen binding protein is a bispecific antibody. Methods of making bispecific antibodies are known in the art. One such method of making a “bispecific,” or “bifunctional” antibody involves the fusion of hybridomas or linking of Fab′ fragments. See, e.g., Songsivilai and Lachmann, 1990, Clin. Erp. Immunol. 79:315-321; Kostelny et al., 1992, 1. Immunol. 148:1547-1553. Another method involves engineering the Fc portion of the heavy chains such as to create “knobs” and “holes” which facilitate heterodimer formation of the heavy chains when co-expressed in a cell. U.S. Pat. No. 7,695,963. Still another method also involves engineering the Fc portion of the heavy chain but uses electrostatic steering to encourage heterodimer formation while discouraging homodimer formation of the heavy chains when co-expressed in a cell. WO 09/089,004, which is incorporated herein by reference in its entirety.

The term “human antibody” includes antibodies having antibody regions such as variable and constant regions or domains which correspond substantially to human germline immunoglobulin sequences known in the art, including, for example, those described by Kabat et al. (1991) (loc. cit.). The human antibodies of the present disclosure may include amino acid residues not encoded by human germline immunoglobulin sequences (e.g., mutations introduced by random or site-specific mutagenesis in vitro or by somatic mutation in vivo), for example in the CDRs, and in particular, in CDR3. The human antibodies can have up to one, two, three, four, five, or more positions replaced with an amino acid residue that is not encoded by the human germline immunoglobulin sequence. The definition of human antibodies as used herein also contemplates fully human antibodies, which include only non-artificially and/or genetically altered human sequences of antibodies as those can be derived by using technologies or systems known in the art, such as for example, phage display technology or transgenic mouse technology, including but not limited to the Xenomouse.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

A “neutralizing antibody” or “inhibitory antibody” or “antagonizing antibody” refers to an antibody that binds to a target molecule and reduces and/or prevents the biological effect of that target molecule. This can be done, for example, by directly blocking a site on the target molecule through which the target molecule interacts with other molecules (e.g. blocking a ligand binding site of a receptor or blocking a receptor binding site on a ligand) or by indirectly blocking a site on the target molecule through which the target molecule interacts with other molecules (such as structural or energetic alterations in the target molecule). In some embodiments, these terms can also denote an antibody that prevents the target molecule to which it is bound from performing a biological function. In assessing the binding and/or specificity of an antibody or immunologically functional fragment thereof, an antibody or fragment can substantially inhibit binding of a target molecule to its binding partner when an excess of antibody reduces the quantity of binding partner bound to the target molecule by at least about 1-20, 20-30%, 30-40%, 40-50%, 50-60%, 60-70%, 70-80%, 80-85%, 85-90%, 90-95%, 95-97%, 97-98%, 98-99%, 99.5%, 99.9% and 100%. In some embodiments, inhibition is complete. The measurement of reduction of binding is done using various assays known to those skilled in the art, (e.g., an in vitro competitive binding assay) and performed using relevant control molecules so that actual inhibition is measured. For example, numerous competition assays are well known in the art, with non-limiting examples being competition ELISA, use of the BiaCore® platform, the Kinexa® platform, or the like. Further examples include: solid phase direct or indirect radioimmunoassay (MA), solid phase direct or indirect enzyme immunoassay (ETA), sandwich competition assay (see, e.g., Stahli et al., 1983, Methods in Enzymology 9:242-253); solid phase direct biotin-avidin ETA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:7-82). Typically, such an assay involves the use of purified antigen bound to a solid surface or cells bearing either of these, an unlabeled test antibody and a labeled reference antibody. In the case of anti-PCSK9 antibodies, such a neutralizing molecule can diminish the ability of PCSK9 to bind the LDLR to lower LDL-C (and/or other lipids, such as non-HDL-C, ApoB, Lp(a), etc.) levels. In some embodiments, the neutralizing ability is characterized and/or described via a competition assay. In some embodiments, the neutralizing ability is described in terms of an IC₅₀ or EC₅₀ value. In some embodiments, the antibodies neutralize by binding to PCSK9 and preventing PCSK9 from binding to LDLR (or reducing the ability of PCSK9 to bind to LDLR). In some embodiments, the antibodies neutralize by binding to PCSK9, and while still allowing PCSK9 to bind to LDLR, preventing or reducing the PCSK9 mediated degradation of LDLR Thus, in some embodiments, a neutralizing antibody can still permit PCSK9/LDLR binding, but will prevent (or reduce) subsequent PCSK9 involved degradation of LDLR. In some embodiments, neutralizing results in the lowering LDL-C (and/or other lipids, such as non-HDL-C, ApoB, Lp(a), etc.).

An antibody is said to “specifically bind” its target antigen when the dissociation constant (K_{D}) is ≤10⁻⁷ M. The antibody specifically binds antigen with “high affinity” when the K_{D} is ≤5×10⁻⁹ M, and with “very high affinity” when the K_{D} is ≤5×10⁻¹⁰ M. In one embodiment, the antibody has a K_{D} of ≤10⁻⁹ M. In one embodiment, the off-rate is ≤1×10⁻⁵. In other embodiments, the antibodies will bind to human PCSK9 with a K_{D} of between about 10⁻⁹ M and 10⁻¹³ M, and in yet another embodiment the antibodies will bind with a K_{D}≤5×10⁻¹⁰. As will be appreciated by one of skill in the art, in some embodiments, any or all of the antibodies can specifically bind to PCSK9.

An antibody is “selective” when it binds to one target more tightly than it binds to a second target.

The term “isolated protein” means that a subject protein (1) is free of at least some other proteins with which it would normally be found, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, and/or (6) does not occur in nature. Typically, an “isolated protein” constitutes at least about 5%, at least about 10%, at least about 25%, or at least about 50% of a given sample. Genomic DNA, cDNA, mRNA or other RNA, of synthetic origin, or any combination thereof can encode such an isolated protein. Preferably, the isolated protein is substantially free from proteins or polypeptides or other contaminants that are found in its natural environment that would interfere with its therapeutic, diagnostic, prophylactic, research or other use.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; Carillo et al., 1988, SIAM J. Applied Math. 48:1073; and Altschul et al. (J Mol Biol. 1990 Oct. 5; 215(3):403-10).

In calculating percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 120, PAM 250 or BLOSum 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSum 62 comparison matrix; Altschul, S. F. 1991, J Mol Biol. 1991 Jun. 5; 219(3): 555-565 for the PAM 120 comparison matrix) is also used by the algorithm.

As used herein, the twenty conventional (e.g., naturally occurring) amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference for any purpose. Stereoisomers (e.g., D-amino acids) of the twenty conventional amino acids, unnatural amino acids such as α-, α-disubstituted amino acids, N-alkyl amino acids, lactic acid, and other unconventional amino acids can also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences.”

Amino acid substitutions can encompass non-naturally occurring amino acid residues, which are typically incorporated by chemical peptide synthesis rather than by synthesis in biological systems. These include peptidomimetics and other reversed or inverted forms of amino acid moieties.

Naturally occurring residues can be divided into classes based on common side chain properties: 1) hydrophobic: norleucine, Met, Ala, Val, Leu, Ile; 2) neutral hydrophilic: Cys, Ser, Thr, Asn, Gin; 3) acidic; Asp, Glu; 4) basic: His, Lys, Arg; 5) residues that influence chain orientation: Gly, Pro; and 6) aromatic: Trp, Tyr, Phe. For example, conservative substitutions in polypeptide molecules described herein (such as antibodies) can involve the exchange of a member of one of these classes for a member of the same class. For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the antigen binding protein or the PCSK9 protein, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157:105-131 (1982). It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One can also identify epitopes from primary amino acid sequences on the basis of hydrophilicity. These regions are also referred to as “epitopic core regions.”

Exemplary amino acid substitutions are set forth in Table 1.0. By way of example, selected exemplary substitutions are shown in the right column.

TABLE 1.0
Amino Acid Substitutions

| Original | Exemplary | Selected Exemplary |
|---|---|---|
| Residues | Substitutions | Substitution |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

As used herein the term “subject” refers to a mammal, including humans, and can be used interchangeably with the term “patient.” A subject or patient can include adults or pediatric subjects, unless indicated otherwise. An adult subject is generally 18 years or older. In some embodiments, an adult subject is between 18 and 90 years old, e.g., between 18 and 85 years old, or between 18 and 80 years old. A pediatric subject refers to a subject younger than 18 years old. In some embodiments, a pediatric subject is 17 years old or younger. In some embodiments, the subject is a pediatric subject 10 to 17 years old. In some embodiments, the subject is a pediatric subject younger than 13 years old. In some embodiments, the subject is a pediatric subject 10 to 17 years old and has HeFH.

The term “treatment” encompasses alleviation of at least one symptom or other embodiment of a disorder, or reduction of disease severity, and the like. A PCSK9 inhibitor and/or one or more other LDL cholesterol-lowering therapy need not effect a complete cure, or eradicate every symptom or manifestation of a disease, to constitute a viable therapeutic agent. As is recognized in the pertinent field, drugs employed as therapeutic agents may reduce the severity of a given disease state, but need not abolish every manifestation of the disease to be regarded as useful therapeutic agents. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient in some embodiments. Certain embodiments of the present disclosure are directed to a method comprising administering to a subject a PCSK9 inhibitor (e.g., an anti-PCSK9 antibody or interfering RNA) in an amount and for a time sufficient to induce a sustained improvement over baseline of an indicator that reflects the severity of the particular disorder.

The term “prevention” encompasses prevention of at least one symptom or other embodiment of a disorder, and the like. A prophylactically administered treatment incorporating an PCSK9 inhibitor and/or one or more other LDL cholesterol-lowering therapy, according to the present disclosure, need not be completely effective in preventing the onset of a condition in order to constitute a viable prophylactic agent. Simply reducing the likelihood that the disease will occur or worsen in a subject, is sufficient in some embodiments. In some embodiments, development of a disease symptom is retarded by therapeutic methods of the present disclosure.

The term “non-HDL cholesterol” encompasses all cholesterol-containing proatherogenic lipoproteins, including LDL cholesterol, very-low-density lipoprotein, intermediate-density lipoprotein, lipoprotein(a), and chylomicron. Non-HDL cholesterol levels are calculated by subtracting HDL cholesterol levels from total cholesterol levels.

### Methods of Treatment

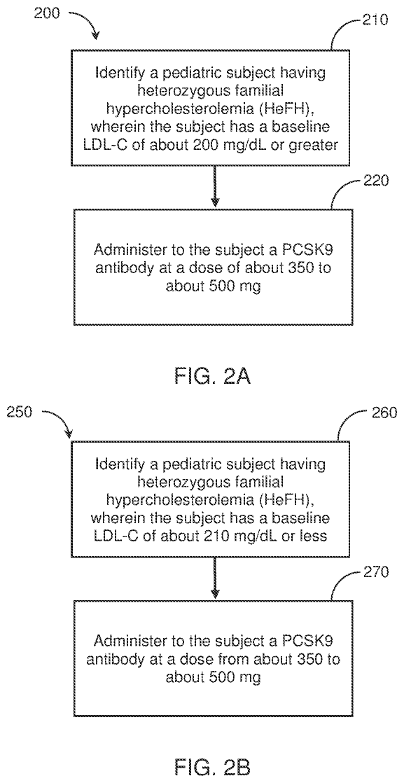

Methods of lowering serum LDL cholesterol (LDL-C) in a subject, e.g., a pediatric subject, having heterozygous familial hypercholesterolemia (HeFH) are provided. With reference to FIG. 2A, a non-limiting example of the present therapeutic methods is described. The method 200 can include identifying 210 a pediatric subject having heterozygous familial hypercholesterolemia (HeFH), wherein the subject has a baseline LDL-C of about 200 mg/dL or greater. The method can further include administering 220 to the subject a PCSK9 antibody at a dose of about 350 to about 500 mg, to thereby lower the subject's LDL-C. In some embodiments, the anti-PCSK9 antibody is administered every four weeks, and the subject's LDL-C is lowered by at least 20%, or by about 20% to about 40%. In some embodiments, the anti-PCSK9 antibody is administered more frequently than every four weeks, e.g., every two weeks, and the subject's LDL-C is lowered by at least 30%, e.g., by at least 45%, or by about 30% to about 80%. In some embodiments, the pediatric subject is also on an additional LDL-cholesterol-lowering therapy (e.g., a statin), as described herein.

With reference to FIG. 2B, a non-limiting example of the present therapeutic methods is described. The method 250 can include identifying 260 a pediatric subject having heterozygous familial hypercholesterolemia (HeFH), wherein the subject has a baseline LDL-C of about 200 mg/dL or less. The method can further include administering 270 to the subject a PCSK9 antibody at a dose of about 350 to about 500 mg, to thereby lower the subject's LDL-C by at least 40%, or by about 400%6 to about 80%. In some embodiments, the baseline LDL-C is less than 208 mg/dL. In some embodiments, the anti-PCSK9 antibody is administered every four weeks, and the subject's LDL-C is lowered by at least about 40%. In some embodiments, the anti-PCSK9 antibody is administered every two weeks, and the subject's LDL-C is lowered by at least about 50%.

In some embodiments, the PCSK9 antibody comprises: a heavy chain variable region (VH) comprising: a CDRH1, CDRH2, and a CDRH3 of a CDRH1, CDRH2, and a CDRH3, respectively, of a VH of evolocumab; and a light chain variable region (VL) comprising: a CDRL1, CDRL2, and a CDRL3 of a CDRL1, CDRL2, and a CDRL3, respectively, of a VL of evolocumab. In some embodiments, the PCSK9 antibody comprises an amino acid sequence at least 90°/% (or at least 91, 92, 93, 94, 95, 96, 97, 98, or 99%) identical to the VH of evolocumab; and a light chain variable region (VL) comprising an amino acid sequence at least 90% (or at least 91, 92, 93, 94, 95, 96, 97, 98, or 99%) identical to the VL of evolocumab. In some embodiments, the PCSK9 antibody comprises: a heavy chain variable region (VH) comprising: a CDRH1, CDRH2, and a CDRH3 of a CDRH1, CDRH2, and a CDRH3, respectively, of a VH of evolocumab; and an amino acid sequence at least 90% (or at least 91, 92, 93, 94, 95, 96, 97, 98, or 99%) identical to the VH of evolocumab; and a light chain variable region (VL) comprising: a CDRL1, CDRL2, and a CDRL3 of a CDRL1, CDRL2, and a CDRL3, respectively, of a VL of evolocumab; and an amino acid sequence at least 90% (or at least 91, 92, 93, 94, 95, 96, 97, 98, or 99%) identical to the VL of evolocumab. In some embodiments, the PCSK9 antibody is evolocumab. The amino acid sequences of the heavy and light chains of evolocumab is shown in FIG. 14.

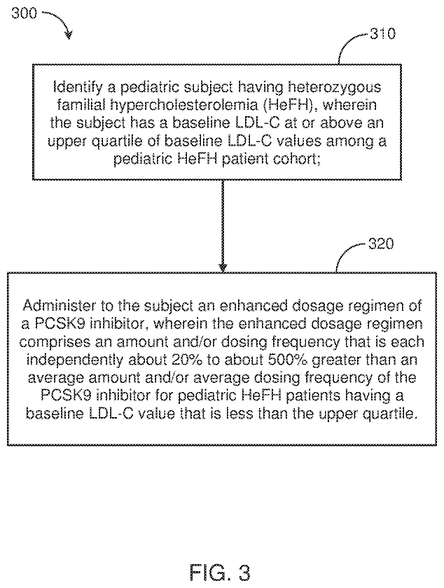

With reference to FIG. 3, a non-limiting example of the present therapeutic methods is described. The method 300 can include identifying 310 a pediatric subject having heterozygous familial hypercholesterolemia (HeFH), wherein the subject has a baseline LDL-C at or above an upper quartile of baseline LDL-C values among a pediatric HeFH patient cohort. The method can further include administering 320 to the pediatric subject an enhanced dosage regimen of a PCSK9 antibody, wherein the enhanced dosage regimen comprises an amount and/or dosing frequency that is each independently about 20% to about 500% greater than an average amount and/or average dosing frequency of the PCSK9 inhibitor for pediatric HeFH patients having a baseline LDL-C value that is less than the upper quartile, whereby the pediatric subject's LDL-C is lowered. In some embodiments, the pediatric subject's baseline LDL-C is about 200 mg/dL or greater.

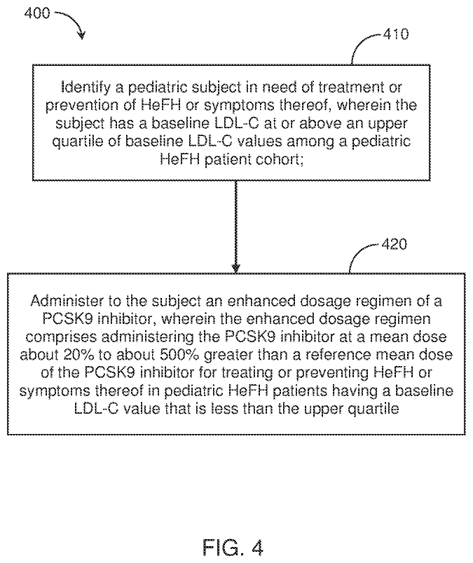

With reference to FIG. 4, a non-limiting example of a method of treating or preventing heterozygous familial hypercholesterolemia (HeFH) or symptoms thereof is provided. The method 400 can include identifying 410 a pediatric subject in need of treatment or prevention of HeFH or symptoms thereof, wherein the pediatric subject has a baseline LDL-C at or above an upper quartile of baseline LDL-C values among a pediatric HeFH patient cohort. Further, the method can include administering 420 to the subject an enhanced dosage regimen of a PCSK9 inhibitor, to thereby treat or prevent the HeFH or symptoms thereof, wherein the enhanced dosage regimen comprises a mean dose about 20% to about 500% greater than a reference mean dose of the PCSK9 inhibitor for treating or preventing HeFH or symptoms thereof in pediatric HeFH patients having a baseline LDL-C value that is less than the upper quartile. In some embodiments, the enhanced dosage regimen comprises a mean dose about 20%, about 30%, about 40%, about 50%, about 60%, about 70%, about 80%, about 90%, about 100%, about 120%, about 140%, about 160%, about 180%, about 200%, about 220%, about 250%, about 300%, about 350%, about 400%, about 450%, about 500% or more, or a percentage within a range defined by any two of the preceding values, greater than the reference mean dose.

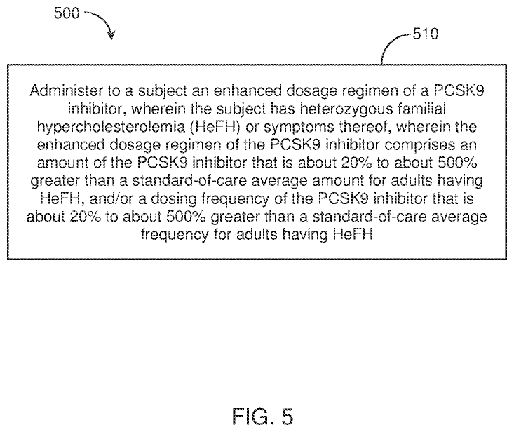

With reference to FIG. 5, a method of lowering serum LDL-cholesterol (LDL-C) in a subject is described. The method 500 can include administering 510 to a subject an enhanced dosage regimen of a PCSK9 inhibitor, wherein the subject has heterozygous familial hypercholesterolemia (HeFH) or symptoms thereof. The enhanced dosage regimen of the PCSK9 inhibitor comprises an amount of the PCSK9 inhibitor that is about 20% to about 500% greater than a standard-of-care average amount for adults having HeFH, and/or a dosing frequency of the PCSK9 inhibitor that is about 20% to about 500% greater than a standard-of-care average frequency for adults having HeFH, whereby the subject's LDL-C is lowered.

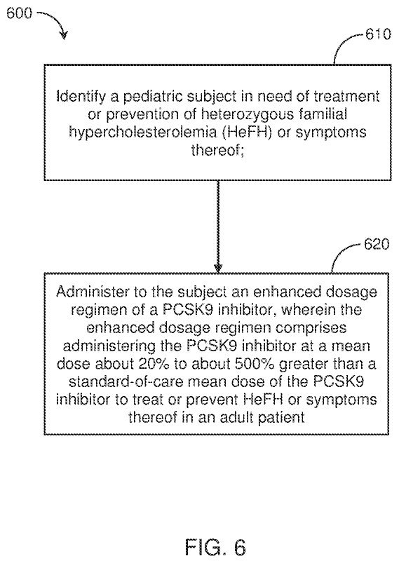

With reference to FIG. 6, a non-limiting example of a method of treating or preventing heterozygous familial hypercholesterolemia (HeFH) or symptoms thereof is described. The method 600 includes identifying 610 a pediatric subject in need of treatment or prevention of heterozygous familial hypercholesterolemia (HeFH) or symptoms thereof. The method can further include administering 620 to the subject an enhanced dosage regimen of a PCSK9 inhibitor, to thereby treat or prevent HeFH or symptoms thereof, wherein the enhanced dosage regimen comprises administering the PCSK9 inhibitor at a mean dose about 20% to about 500% greater than a standard-of-care mean dose of the PCSK9 inhibitor to treat or prevent HeFH or symptoms thereof in an adult patient.

Also provided herein is a method of lowering serum LDL cholesterol (LDL-C), comprising: administering to a subject a PCSK9 inhibitor, wherein the subject has heterozygous familial hypercholesterolemia, wherein the subject is a pediatric subject, wherein the PCSK9 inhibitor is administered in an amount that is at least as effective as 420 mg of evolocumab, wherein the PCSK9 inhibitor is administered at a frequency of every two weeks or more, whereby the subject's LDL-C is reduced by more than 30%. The efficacy of the PCSK9 inhibitor in some embodiments is based on the extent to which the subject's serum LDL-C, or serum total cholesterol, is reduced by administering the PCSK9 inhibitor to HeFH patients. In some embodiments, the relative efficacy of the PCSK9 inhibitor and 420 mg of evolocumab is determined in adult or pediatric patients having HeFH. In some embodiments, the PCSK9 inhibitor is administered in an amount that is at least as effective as 420 mg of evolocumab administered to an adult patient with HeFH. In some embodiments, the PCSK9 inhibitor is administered in an amount that is at least as effective as 420 mg of evolocumab administered to a pediatric patient having a baseline LDL-C level that is less than an upper quartile of baseline LDL-C values among a pediatric HeFH patient cohort, as described herein. In some embodiments, the subject has a baseline LDL-C that is at or above the upper quartile. In some embodiments, the PCSK9 inhibitor is at least 70%, at least 80%, at least 85%, at least 90%, at least 95%, at least 97%, or about 100% as effective as 420 mg of evolocumab.

In some embodiments, a PCSK9 inhibitor of the present disclosure (e.g., anti-PCSK9 antibody, inhibitory nucleic acid or small molecule inhibitor) is administered to the subject at least every 4 weeks, every 3 weeks, every 2 weeks, or every week or more frequently, or at a frequency within a range defined by any two of the preceding values. In some embodiments, the PCSK9 inhibitor, e.g., anti-PCSK9 antibody, inhibitory nucleic acid, or small molecule inhibitor, is administered to the subject every 25, 24, 23, 22, 21, 20, 19, 18, 17, 16, 15, 14, 13, 12, 11, 10, 9, 8, 7, 6, 5, 4, or 3 days, or more frequently. In some embodiments, the PCSK9 inhibitor, e.g., anti-PCSK9 antibody, is administered every two to four weeks. In some embodiments, the PCSK9 inhibitor, e.g., anti-PCSK9 antibody, is administered every two weeks. In some embodiments, the PCSK9 inhibitor, e.g., anti-PCSK9 antibody, is administered every four weeks. In some embodiments, the subject has a baseline LDL-C of about 200 mg/dL or greater, and the PCSK9 inhibitor, e.g., anti-PCSK9 antibody, is administered every two weeks. In some embodiments, the subject has a baseline LDL-C of about 200 mg/dL or less, and the PCSK9 inhibitor, e.g., anti-PCSK9 antibody, is administered every four weeks.

In some embodiments, a PCSK9 antibody of the present disclosure is administered to the subject at an amount, e.g., amount per dose, of at least 10, 20, 30, 40, 50, 60, 70, 80, 90, 100, 120, 140, 160, 180, 200, 220, 240, 260, 280, 300, 320, 340, 360, 380, 400, 420, 440, 460, 480, 490, 500 mg or more, or at an amount within a range defined by any two of the preceding values. In some embodiments, the PCSK9 antibody is administered at an amount, e.g., amount per dose, of about 420 mg. In some embodiments, the PCSK9 antibody is administered at an amount, e.g., amount per dose, of about 490 mg.

In some embodiments, the PCSK9 inhibitor, e.g., anti-PCSK9 antibody or inhibitory nucleic acid, is administered according to an enhanced dosage regimen. In some embodiments, the enhanced dosage regimen includes a dosing frequency and/or amount that is increased compared to a reference dosage regimen. In some embodiments, the reference dosage regimen is a dosage regimen for pediatric HeFH patients having a baseline LDL-C value that is less than an upper quartile, e.g., less than a median, of baseline LDL-C values among a pediatric HeFH patient cohort, as disclosed herein. In some embodiments, the reference dosage regimen is a dosage regimen for treating or preventing HeFH or symptoms thereof in pediatric HeFH patients having a baseline LDL-C value that is less than an upper quartile, e.g., less than a median, of baseline LDL-C values among a pediatric HeFH patient cohort, as disclosed herein. In some embodiments, the reference dosage regimen is a standard-of-care dosage regimen for the PCSK9 inhibitor for an adult patient with HeFH. In some embodiments, the PCSK9 inhibitor is approved by a government regulatory agency (e.g., FDA-approved) for lowering LDL-C in a human patient. In some embodiments, a standard-of-care dosage regimen of a government regulatory agency-approved PCSK9 inhibitor is a dosage regimen provided in the government regulatory agency-approved label for the PCSK9 inhibitor. In some embodiments, the reference dosage regimen is a dosage regimen in a government regulatory agency-approved label (e.g., FDA-approved label) for the PCSK9 inhibitor. In some embodiments, the reference dosage regimen includes an average dosing frequency and/or average amount in a government regulatory agency-approved label (e.g., FDA-approved label) for the PCSK9 inhibitor.

In some embodiments, the enhanced dosage regimen includes a dosing frequency and/or an amount, e.g., amount per dose, of the PCSK9 inhibitor that is greater than a reference dosing frequency and/or reference amount of the PCSK9 inhibitor. In some embodiments, the enhanced dosage regimen includes a dosing frequency that is greater than a reference dosing frequency. In some embodiments, the enhanced dosage regimen includes a dosing frequency that is at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.5 fold, at least 3 fold, at least 3.2 fold, at least 3.5 fold, at least 4 fold, at least 5 fold or more, or a fold amount within a range defined by any two of the preceding values, greater than a reference dosing frequency. In some embodiments, the enhanced dosage regimen includes a dosing frequency that is at least 20%, at least 30%, at least 40%, at least 50%, at least 60%, at least 70%, at least 80%, at least 90%, at least 100%, at least 120%, at least 140%, at least 160%, at least 180%, at least 200%, at least 250%, at least 300%, at least 400%, at least 500% or more, or a percentage within a range defined by any two of the preceding values, greater than a reference dosing frequency.

In some embodiments, the reference dosing frequency is about once every week, every 2 weeks, every 3 weeks, every 4 weeks, every 5 weeks, every 6 weeks, every 7 weeks, every 8 weeks, every 12 weeks, every month, every 2 months, every 3 months, every 4 months, every 6 months or more, or a frequency within a range defined by any two of the preceding values. In some embodiments, the reference dosing frequency is based on a dosage regimen of the PCSK9 inhibitor for pediatric HeFH patients having a baseline LDL-C value that is less than an upper quartile, e.g., less than a median, of baseline LDL-C values among a pediatric HeFH patient cohort, as disclosed herein. The reference dosing frequency, in some embodiments, is an average dosing frequency of the PCSK9 inhibitor for pediatric HeFH patients having a baseline LDL-C value that is less than an upper quartile, e.g., less than a median, of baseline LDL-C values among a pediatric HeFH patient cohort, as disclosed herein. In some embodiments, the average dosing frequency of the PCSK9 inhibitor for pediatric HeFH patients having a baseline LDL-C value that is less than the upper quartile, e.g., less than the median, is about once every week, every 2 weeks, every 3 weeks, every 4 weeks, every 5 weeks, every 6 weeks, every 7 weeks, every 8 weeks, every month, every 2 months, every 3 months, every 4 months, every 6 months or more, or a frequency within a range defined by any two of the preceding values.

In some embodiments, the enhanced dosage regimen includes an amount, e.g., amount per dose, that is greater than a reference amount, e.g., reference amount per dose. In some embodiments, the enhanced dosage regimen includes an amount, e.g., amount per dose, that is at least 20%, at least 30%, at least 40%, at least 50%, at least 60%, at least 70%, at least 80%, at least 90%, at least 100%, at least 120%, at least 140%, at least 160%, at least 180%, at least 200%, at least 250%, at least 300%, at least 400%, at least 500% or more, or a percentage within a range defined by any two of the preceding values, greater than the reference amount.

In some embodiments, the reference amount, e.g., reference amount per dose, is at least 10, 20, 30, 40, 50, 60, 70, 80, 90, 100, 120, 140, 160, 180, 200, 220, 240, 260, 280, 300, 320, 340, 360, 380, 400, 420, 440, 460 mg, 480 mg, 500 mg, 550 mg, 600 mg, 650 mg, 700 mg, 750 mg, 800 mg or more, or an amount within a range defined by any two of the preceding values. In some embodiments, the reference amount, e.g., reference amount per dose, is based on a dosage regimen of the PCSK9 inhibitor for pediatric HeFH patients having a baseline LDL-C value that is less than an upper quartile, e.g., less than a median, of baseline LDL-C values among a pediatric HeFH patient cohort, as disclosed herein. The reference amount, in some embodiments, is an average amount of the PCSK9 inhibitor for pediatric HeFH patients having a baseline LDL-C value that is less than an upper quartile, e.g., less than a median, of baseline LDL-C values among a pediatric HeFH patient cohort, as disclosed herein. In some embodiments, the average amount of the PCSK9 inhibitor for pediatric HeFH patients having a baseline LDL-C value that is less than the upper quartile, e.g., less than the median, is at least 10, 20, 30, 40, 50, 60, 70, 80, 90, 100, 120, 140, 160, 180, 200, 220, 240, 260, 280, 300, 320, 340, 360, 380, 400, 420, 440, 460 mg, 480 mg, 500 mg, 550 mg, 600 mg, 650 mg, 700 mg, 750 mg, 800 mg or more, or an amount within a range defined by any two of the preceding values.

In some embodiments, the enhanced dosage regimen includes a dosing frequency that is increased compared to a standard-of-care average dosing frequency of the PCSK9 inhibitor, e.g., for an adult with HeFH. In some embodiments, the dosing frequency of the PCSK9 inhibitor administered to the subject is about 15%, about 20%, about 30%, about 40%, about 50%, about 60%, about 70%, about 80%, about 90%, about 100%, about 120%, about 140%, about 160%, about 180° %, about 200%, about 250%, about 300%, about 350%, about 400%, about 450%, about 500% or more, or a percentage within a range defined by any two of the preceding values, greater than the standard-of-care average dosing frequency. In some embodiments, the dosing frequency of the PCSK9 inhibitor administered to the subject is about 15% to about 400% greater than the standard-of-care average dosing frequency. In some embodiments, the standard-of-care average dosing frequency is about once every week, every 2 weeks, every 3 weeks, every 4 weeks, every 5 weeks, every 6 weeks, every 7 weeks, every 8 weeks, every month, every 2 months, every 3 months, every 4 months, every 6 months or more, or a frequency within a range defined by any two of the preceding values.

In some embodiments, the enhanced dosage regimen includes an amount, e.g., amount per dose, of the PCSK9 inhibitor that is greater than a standard-of-care average amount of the PCSK9 inhibitor, e.g., for an adult with HeFH. In some embodiments, the amount of the PCSK9 inhibitor administered to the subject is about 5%, about 10%, about 15%, about 20%, about 25%, about 30%0, about 35%, about 40%, about 45%, about 50%, about 55%, about 60%, about 65%, about 70%, about 75%, about 800, about 85%, about 90%, about 95%, about 100%, or more, or a percentage within a range defined by any two of the preceding values, greater than the standard-of-care average amount. In some embodiments, the amount of the PCSK9 inhibitor administered to the subject is about 5% to about 100% greater than the standard-of-care average amount. In some embodiments, the standard-of-care average amount is at least 10, 20, 30, 40, 50, 60, 70, 80, 90, 100, 120, 140, 160, 180, 200, 220, 240, 260, 280, 300, 320, 340, 360, 380, 400, 420, 440, 460 mg or more, or an amount within a range defined by any two of the preceding values.

In some embodiments, the enhanced dosage regimen includes a mean dose of the PCSK9 inhibitor that is greater than a reference mean dose of the PCSK9 inhibitor (e.g., for pediatric HeFH patients having a baseline LDL-C value that is less than an upper quartile, e.g., less than a median, of baseline LDL-C values among a pediatric HeFH patient cohort; for treating or preventing HeFH or symptoms thereof in pediatric HeFH patients having a baseline LDL-C value that is less than an upper quartile, e.g., less than a median, of baseline LDL-C values among a pediatric HeFH patient cohort, as disclosed herein). The “mean dose” as used herein refers to an amount of a therapeutic agent, e.g., PCSK9 inhibitor, administered to the subject per unit time (e.g., mg/day, mg/week, mg/month, etc.). In some embodiments, the enhanced dosage regimen includes a mean dose of the PCSK9 inhibitor that is greater than a standard-of-care mean dose of the PCSK9 inhibitor, e.g., for an adult with HeFH. In some embodiments, the enhanced dosage regimen includes a mean dose of at least 140, 160, 180, 200, 220, 240, 260, 280, 300, 320, 340, 360, 380, 400, 420, 440, 460, 480, 500, 520, 550, 600, 620, 650, 700, 720, 750, 800, 820, 850, 900, 920, 950, 1,000, 1,100, 1,200, 1,300, 1,400, 1,500 mg/month or more, or a mean dose within a range defined by any two of the preceding values. In some embodiments, the enhanced dosage regimen includes a mean dose that is about 20%, about 30%, about 40%, about 50%, about 60%, about 70%, about 80%, about 90%, about 100%, about 120%, about 140%, about 160%, about 180%, about 200%, about 220%, about 250%, about 300%, about 350%, about 400%, about 450%, about 500% or more, or a percentage within a range defined by any two of the preceding values, greater than the reference mean dose. In some embodiments, the reference mean dose is at least 70, 80, 90, 100, 120, 140, 160, 180, 200, 220, 240, 260, 280, 300, 320, 340, 360, 380, 400, 420, 440, 460, 480, 500, 520, 550, 600, 620, 650, 700, 720, 750, 800, 820, 850, or 900 mg/month or more, or a mean dose within a range defined by any two of the preceding values.

In some embodiments, the enhanced dosage regimen includes a dosing frequency and/or amount that is greater than a reference dosing frequency and/or amount (e.g., a dosing frequency and/or amount for pediatric HeFH patients having a baseline LDL-C value that is less than an upper quartile, e.g., less than a median, of baseline LDL-C values among a pediatric HeFH patient cohort; a dosing frequency and/or amount for treating or preventing HeFH or symptoms thereof in pediatric HeFH patients having a baseline LDL-C value that is less than an upper quartile, e.g., less than a median, of baseline LDL-C values among a pediatric HeFH patient cohort), as disclosed herein.

In some embodiments, at least 350, 360, 370, 380, 390, 400, 410, 420, 430, 440, 450, 460, 470, 480, 490, 500 mg of a PCSK9 inhibitor (such as a neutralizing antibody) is administered, per dose, to the pediatric subject. In some embodiments, evolocumab is administered in an amount of at least 350 mg, for example, at least 370 mg, at least 390 mg, at least 400 mg, at least 420 mg, at least 440 mg, at least 450 mg, at least 460 mg, at least 470 mg, at least 480 mg or about 490 mg per dose. In some embodiments, the amount of the anti-PCSK9 neutralizing antibody administered is at least 350 mg, for example, at least 370 mg, at least 390 mg, at least 400 mg, at least 420 mg, at least 440 mg, at least 450 mg, at least 460 mg, at least 470 mg, at least 480 mg or about 490 mg per dose. In some embodiments, the amount of the anti-PCSK9 antibody administered is at least 40 mg, for example, at least 80 mg, at least 150 mg, at least 200 mg, at least 350 mg, at least 400 mg, at least 450 mg, at least 480 mg or about 490 mg, or an amount in a range defined by any two of the preceding values.

The pediatric HeFH patient cohort can be any suitable group of patients who are under the age of 18 and have been diagnosed with HeFH. In some embodiments, the cohort is a representative subset of a general population of pediatric patients having or diagnosed with HeFH. In some embodiments, the cohort includes about 20, 25, 30, 35, 40, 45, 50, 55, 60, 65, 70, 75, 80, 85, 90, 95, 100, 105, 110, 115, 120, 125, 130, 140, 150, 160, 170, 180 190, 200, 250, 300, 350, 400, 450, 500, 600, 700, 800, 900, 1,000, 1,200, 1,400, 1,600, 1,800, 2,000, 2,500, 3,000 or more patients, or a number of patients within a range defined by any two of the preceding values. In some embodiments, the cohort includes at least 25 pediatric HeFH patients. In some embodiments, the cohort includes about 105 or more pediatric HeFH patients. The patients of the cohort can be diagnosed with HeFH using any suitable measure, as described herein. In some embodiments, the average age of the cohort is about 9, 10, 11, 12, 13, 14, 15, 16 years old, or an average age in a range defined by any two of the preceding values. In some embodiments, a pediatric patient in the cohort is at least 8, 9, 10, 11, or 12 years old. In some embodiments, at least 25%, about 30%, about 35%, about 40%, about 45%, about 50%, about 55%, about 60%, about 65%, about 70%, or about 75%, or a percentage in a range defined by any two of the preceding values, of the patients in the cohort are younger than 14 years old. In some embodiments, at least 30%, about 35%, about 40%, about 45%, about 500%6, about 55%, about 60%, about 65% or about 70%, or a percentage in a range defined by any two of the preceding values, of the cohort is male. In some embodiments, at least 10%, about 15%, about 20%, about 25%, about 30%, about 35%, about 40%, about 45%, about 500%6, about 55%, about 60%, about 65% about 70%, about 75%, about 80%, about 85%, about 90%, about 95%, or a percentage in a range defined by any two of the preceding values, of the cohort is Caucasian. In some embodiments, at least 10%, about 15%, about 20%, about 25%, about 30%, about 35%, about 40%, about 45%, about 50%, about 55%, about 60%, about 65% about 70%, about 75%, about 80%, about 85%, about 90%, about 95%, or a percentage in a range defined by any two of the preceding values, of the cohort is of the same race as the race of the pediatric subject being administered the PCSK9 inhibitor (e.g., anti-PCSK9 antibody or inhibitory nucleic acid).

In some embodiments, the pediatric HeFH patient cohort has a median baseline LDL-C of about 150 mg/dL, about 155 mg/dL, about 160 mg/dL, about 165 mg/dL, about 170 mg/dL, about 175 mg/dL, about 180 mg/dL, about 185 mg/dL, about 190 mg/dL, about 195 mg/dL, about 200 mg/dL, about 205 mg/dL, about 210 mg/dL, about 215 mg/dL, about 220 mg/dL, about 225 mg/dL, or about 230 mg/dL, or a median baseline LDL-C in a range defined by any two of the preceding values. In some embodiments, the cohort has a median baseline LDL-C of about 160 mg/dL to about 190 mg/dL. In some embodiments, the cohort has a median baseline LDL-C of 173 mg/dL. In some embodiments, the cohort has an upper quartile baseline LDL-C of about 180 mg/dL, about 185 mg/dL, about 190 mg/dL, about 195 mg/dL, about 200 mg/dL, about 205 mg/dL, about 210 mg/dL, about 215 mg/dL, about 220 mg/dL, about 225 mg/dL, about 230 mg/dL, about 235 mg/dL, about 240 mg/dL, about 245 mg/dL, about 250 mg/dL, about 255 mg/dL, or about 260 mg/dL, or an upper quartile baseline LDL-C in a range defined by any two of the preceding values. In some embodiments, the cohort has an upper quartile baseline LDL-C of about 190 mg/dL to about 220 mg/dL. In some embodiments, the cohort has an upper quartile baseline LDL-C of about 200 mg/dL. In some embodiments, the cohort has an upper quartile baseline LDL-C of about 210 mg/dL. In some embodiments, the cohort has an upper quartile baseline LDL-C of 208 mg/dL. In some embodiments, the cohort has a lower quartile baseline LDL-C of about 135 mg/dL, about 140 mg/dL, about 145 mg/dL, about 150 mg/dL, about 155 mg/dL, about 160 mg/dL, or about 165 mg/dL, or an lower quartile baseline LDL-C in a range defined by any two of the preceding values. In some embodiments, the cohort has an upper quartile baseline LDL-C of 154 mg/dL.

The cohort in some embodiments has an average baseline LDL-C of about 150, about 155, about 160, about 165, about 170, about 175, about 180, about 185, about 190, about 195, about 200 mg/dL, about 205 mg/dL, about 210 mg/dL, about 215 mg/dL, about 220 mg/dL, about 225 mg/dL, or about 230 mg/dL, or an average baseline LDL-C in a range defined by any two of the preceding values. In some embodiments, a pediatric patient in the cohort has a baseline LDL-C of about 110 mg/dL or more, e.g., about 115 mg/dL or more, about 120 mg/dL or more, about 125 mg/dL or more, about 130 mg/dL or more, about 135 mg/dL or more, about 140 mg/dL or more, about 145 mg/dL or more, including about 150 mg/dL or more. In some embodiments, a pediatric patient in the cohort has a baseline LDL-C of at least 130 mg/dL. In some embodiments, a pediatric patient in the cohort has a baseline LDL-C in a range of about 90 mg/dL to about 550 mg/dL, e.g., about 100 mg/dL to about 500 mg/dL, about 110 mg/dL to about 450 mg/dL, about 110 mg/dL to about 400 mg/dL, about 120 mg/dL to about 350 mg/dL, about 130 mg/dL to about 300 mg/dL, including about 130 mg/dL to about 275 mg/dL. In some embodiments, a pediatric patient in the cohort has a baseline (e.g., fasting) triglyceride of about 600 mg/dL or less, e.g., about 550 mg/dL or less, about 500 mg/dL or less, about 475 mg/dL or less, about 450 mg/dL or less, about 425 mg/dL or less, about 400 mg/dL or less, about 375 mg/dL or less, including about 350 mg/dL or less, or a baseline triglyceride within a range defined by any two of the preceding values.

In some embodiments, the cohort has a median baseline non-HDL-C of about 150 mg/dL, about 155 mg/dL, about 160 mg/dL, about 165 mg/dL, about 170 mg/dL, about 175 mg/dL, about 180 mg/dL, about 185 mg/dL, about 190 mg/dL, about 195 mg/dL, about 200 mg/dL, about 205 mg/dL, about 210 mg/dL, about 215 mg/dL, about 220 mg/dL, about 225 mg/dL, about 230 mg/dL, about 235 mg/dL, about 240 mg/dL, about 250 mg/dL or about 260 mg/dL or a median baseline non-HDL-C in a range defined by any two of the preceding values. In some embodiments, the cohort has an upper quartile baseline non-HDL-C of about 190 mg/dL, about 195 mg/dL, about 200 mg/dL, about 205 mg/dL, about 210 mg/dL, about 215 mg/dL, about 220 mg/dL, about 225 mg/dL, about 230 mg/dL, about 235 mg/dL, about 240 mg/dL, about 245 mg/dL, about 250 mg/dL, about 255 mg/dL, about 260 mg/dL, about 265 mg/dL, about 270 mg/dL, about 275 mg/dL, about 280 mg/dL, about 290 mg/dL, or an upper quartile baseline non-HDL-C in a range defined by any two of the preceding values. In some embodiments, the cohort has a lower quartile baseline non-HDL-C of about 135 mg/dL, about 140 mg/dL, about 145 mg/dL, about 150 mg/dL, about 155 mg/dL, about 160 mg/dL, about 165 mg/dL, about 170 mg/dL, about 175 mg/dL, about 180 mg/dL, about 185 mg/dL, or an lower quartile baseline non-HDL-C in a range defined by any two of the preceding values.

The cohort in some embodiments has an average baseline non-HDL-C of about 150, about 155, about 160, about 165, about 170, about 175, about 180, about 185, about 190, about 195, about 200 mg/dL, about 205 mg/dL, about 210 mg/dL, about 215 mg/dL, about 220 mg/dL, about 225 mg/dL, about 230 mg/dL, about 235 mg/dL, about 240 mg/dL, about 245 mg/dL, about 250 mg/dL, or an average baseline non-HDL-C in a range defined by any two of the preceding values.

Patients in the cohort in some embodiments has one or more cardiovascular risk factors, including, without limitation, hypertension, low HDL-C, current cigarette smoking, type II diabetes, and family history of premature coronary heart disease (CHD). In some embodiments, at least 10%, about 15%, about 20%, about 25%, about 30%0, about 35%, about 40%0, about 45%, about 50%, about 55%, or about 60%, or a percentage in a range defined by any two of the preceding values, of patients in the cohort have low HDL-C (HDL-C of, e.g., about 60 mg/dL or less, about 50 mg/dL or less, or about 40 mg/dL or less). In some embodiments, at least 10%, about 15%, about 20%, about 25%, about 30%, about 35%, about 40%, about 45%, about 50%, about 55%, or about 60%, or a percentage in a range defined by any two of the preceding values, of patients in the cohort have a family history of premature CHD.

Patients in the cohort in some embodiments has received, or is receiving one or more other LDL-cholesterol lowering therapy (e.g., a LDL-cholesterol lowering therapy that is not a PCSK9 inhibitor therapy, such as statin therapy). In some embodiments, at least 50%, about 60%, about 70%, about 75%, about 80%, about 85%, about 90%0, about 95%, about 95% or more, or a percentage in a range defined by any two of the preceding values, of patients in the cohort are taking a statin. In some embodiments, all patients in the cohort are taking a statin. In some embodiments, at least 1%, about 5%, about 10%, about 15%, about 20%, about 25%, about 30%, about 35%, or more, or a percentage in a range defined by any two of the preceding values, of patients in the cohort are receiving a high intensity statin therapy. In some embodiments, at least 209%, about 25%, about 30%, about 35%, about 40%, about 45%, about 50%, about 55%, about 60%, about 65%, about 70%, about 75%, about 80%, about 85%, about 90% or more, or a percentage in a range defined by any two of the preceding values, of patients in the cohort are receiving a moderate intensity statin therapy. In some embodiments, all patients in the cohort are taking a statin. In some embodiments, at least 1%, about 5%, about 10%, about 15%, about 20%, about 25%, about 30%, about 35%, or more, or a percentage in a range defined by any two of the preceding values, of patients in the cohort are receiving a low intensity statin therapy. In some embodiments, In some embodiments, at least 1%, about 5%, about 10%, about 15%, about 20%, about 25%, about 30%, about 35%, or more, or a percentage in a range defined by any two of the preceding values, of patients in the cohort are receiving Ezetimibe.

In some embodiments, a subject or patient being administered a PCSK9 inhibitor, according to methods of the present disclosure, is a pediatric subject or patient. A pediatric subject is generally under 18 years old (or 17 years old or younger). In some embodiments, a pediatric subject is between 8 and 17 years old, e.g., between 9 and 17 years old, between 10 and 17 years old, between 11 and 17 years old, including between 12 and 17 years old. In some embodiments, a pediatric subject is between 10 and 17 years old. As used herein, “between” is used inclusive of the number of years defining the range. Thus, a subject “between 10 and 17 years old” includes subjects who are 10 years old or older, and younger than 18 years old, and includes, e.g., a subject who is 17 years and 11 months old).

A subject for administering a PCSK9 inhibitor, according to methods of the present disclosure, may be identified based on one or more of a variety of criteria. In some embodiments, a subject is diagnosed as having HeFH based on one or more clinical measures. Suitable clinical measures include, without limitation, blood biomarker levels (e.g., total cholesterol, LDL cholesterol, and other lipid levels), physical symptoms of HeFH (e.g., arcus corneae, xanthelasma, tendon xanthomas, or tuberous xanthomas), history of coronary heart disease (CHD), and family history. In some embodiments, a subject has, or is identified as having, one or more genetic mutations associated with HeFH. In some embodiments, the subject has, or is identified as having, a mutation associated with HeFH in, without limitation, LDLR, APOB, and/or PCSK9. In some embodiments, the subject has, or is identified as having, one, two, three or more mutations associated with HeFH. In some embodiments, the subject has, or is identified as having, at least one mutation associated with HeFH in one, two, three or more genes implicated in HeFH (e.g., LDLR, APOB, PCSK9). In some embodiments, the subject has, or is identified as having, compound HeFH. In some embodiments, the methods of the present disclosure include diagnosing and/or genotyping the subject to determine the pediatric subject has HeFH, including, without limitation, compound HeFH.

In some embodiments, the methods of the present disclosure include measuring the baseline LDL-C of the pediatric subject.

In some embodiments, the subject being administered the PCSK9 inhibitor (e.g., anti-PCSK9 antibody or inhibitory nucleic acid) has, or is identified as having, a baseline LDL-C that is at or above an upper quartile of baseline LDL-C levels among a cohort of pediatric HeFH patients, as described herein. In some embodiments, the subject has, or is identified as having, a baseline LDL-C of about 200 mg/dL or greater, e.g., about 210 mg/dL or greater, about 220 mg/dL or greater, about 230 mg/dL or greater, about 240 mg/dL or greater, about 250 mg/dL or greater, about 260 mg/dL or greater, about 270 mg/dL or greater, about 280 mg/dL or greater, about 290 mg/dL or greater, including about 300 mg/dL or greater. In some embodiments, the subject has, or is identified as having, a baseline LDL-C in a range of about 200 mg/dL to about 550 mg/dL, e.g., about 200 mg/dL to about 550 mg/dL, about 200 mg/dL to about 500 mg/dL, about 200 mg/dL to about 450 mg/dL, about 200 mg/dL to about 400 mg/dL, about 200 mg/dL to about 350 mg/dL, about 200 mg/dL to about 300 mg/dL, about 200 mg/dL to about 230 mg/dL, including about 200 mg/dL to about 275 mg/dL. In some embodiments, the subject has, or is identified as having, a baseline LDL-C of 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 223, 224, 225, 226, 227, 228, 229, 230 mg/dL or a baseline LDL-C greater than any one of the preceding values. In some embodiments, the subject has, or is identified as having, a baseline LDL-C of 208 mg/dL or more.

In some embodiments, the subject being administered the PCSK9 inhibitor (e.g., anti-PCSK9 antibody or inhibitory nucleic acid) has, or is identified as having, a baseline LDL-C that is at or below an upper quartile, e.g., below a median, of baseline LDL-C levels among a cohort of pediatric HeFH patients, as described herein. In some embodiments, the subject has, or is identified as having, a baseline LDL-C of about 210 mg/dL or less, e.g., about 200 mg/dL or less, about 190 mg/dL or less, about 180 mg/dL or less, about 170 mg/dL or less, about 160 mg/dL or less, about 150 mg/dL or less, about 140 mg/dL or less, about 130 mg/dL or less, about 120 mg/dL or less, including about 110 mg/dL or less. In some embodiments, the subject has, or is identified as having, a baseline LDL-C of 208 mg/dL or less. In some embodiments, the subject has, or is identified as having, a baseline LDL-C of 173 mg/dL or less. In some embodiments, the subject has, or is identified as having, a baseline LDL-C in a range of about 80 mg/dL to about 210 mg/dL, e.g., about 90 mg/dL to about 210 mg/dL, about 100 mg/dL to about 210 mg/dL, about 110 mg/dL to about 210 mg/dL, about 120 mg/dL to about 210 mg/dL, about 130 mg/dL to about 210 mg/dL, about 130 mg/dL to about 200 mg/dL, about 130 mg/dL to about 190 mg/dL, about 130 mg/dL to about 180 mg/dL, including about 130 mg/dL to 173 mg/dL. In some embodiments, the subject has, or is identified as having, a baseline LDL-C between 130 mg/dL and 173 mg/dL. In some embodiments, the subject has, or is identified as having, a baseline LDL-C between 130 mg/dL and 208 mg/dL. In some embodiments, the subject has, or is identified as having, a baseline LDL-C of 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208 mg/dL or a baseline LDL-C less than any one of the preceding values. In some embodiments, the subject has, or is identified as having, a baseline LDL-C of 208 mg/dL or less. In some embodiments, the methods of the present disclosure include measuring the baseline LDL-C of the pediatric subject.

In some embodiments, the methods of the present disclosure lowers the subject's LDL-C by at least 30%. In some embodiments, the subject's LDL-C is lowered by about 30% to about 80%. In some embodiments, the subject's LDL-C is reduced by at least 200%, at least 30%, at least 35%, at least 40%, at least 45%, at least 50%, at least 55%, at least 60%, at least 65%, at least 70%, at least 75%, at least 80%, or more, or by a percentage in a range defined by any two of the preceding values. In some embodiments, the subject has a baseline LDL-C of about 200 mg/dL or greater, and the subject's LDL-C is lowered by at least 20%, at least 30%, at least 40%, about 30% to about 50%/6, about 20% to about 50%, about 20% to about 80%, about 30% to about 50%, or about 30% to about 80%. In some embodiments, the subject has a baseline LDL-C of about 200 mg/dL or greater, the PCSK9 inhibitor, e.g., anti-PCSK9 antibody, is administered every four weeks, and wherein the subject's LDL-C is lowered by at least 20%. In some embodiments, the subject has a baseline LDL-C of about 200 mg/dL or greater, the PCSK9 inhibitor, e.g., anti-PCSK9 antibody, is administered every two weeks, and wherein the subject's LDL-C is lowered by at least 30%.

In some embodiments, the pediatric subject has a baseline LDL-C of about 210 mg/dL or less, and the subject's LDL-C is lowered by at least 40%, at least 45%, at least 50%, at least 60%, about 40% to about 60%, about 40% to about 80%, about 50% to about 60%, or about 50% to about 80%. In some embodiments, the pediatric subject has a baseline LDL-C of about 210 mg/dL or less, and the subject's LDL-C is lowered by at least 45%. In some embodiments, the subject has a baseline LDL-C of about 210 mg/dL or less, the PCSK9 inhibitor, e.g., anti-PCSK9 antibody, is administered every four weeks, and the subject's LDL-C is lowered by at least 40%. In some embodiments, the subject has a baseline LDL-C of about 210 mg/dL or less, the PCSK9 inhibitor, e.g., anti-PCSK9 antibody, is administered every two weeks, and the subject's LDL-C is lowered by at least 50%.

In some embodiments, the reduction in the pediatric subject's LDL-C is at least 40%, at least 50%, at least 55%, at least 60%, at least 65%, at least 70%, at least 75%, at least 80%, at least 85%, at least 90%, at least 95%, or about 100%, or a percentage within a range defined by any two of the preceding values, of the reduction in LDL-C achieved in a reference patient population (e.g., pediatric HeFH patients having a baseline LDL-C value that is less than the upper quartile or the median of baseline LDL-C values among a pediatric HeFH patient cohort; adult HeFH patients) that is administered a PCSK9 inhibitor therapy under a reference dosage regimen of the PCSK9 inhibitor (e.g., a dosage regimen for pediatric HeFH patients in the reference patient population; a standard-of-care dosage regimen for the reference patient population; a dosage regimen under a government regulatory agency-approved label, etc.). In some embodiments, the reduction in the pediatric subject's LDL-C is at least 40%, at least 50%, at least 55%, at least 609%, at least 65%, at least 70%, at least 75%, at least 80%, at least 85%, at least 90%, at least 95%, or about 100%, or a percentage within a range defined by any two of the preceding values, of the reduction in LDL-C achieved in a reference patient population (e.g., pediatric HeFH patients having a baseline LDL-C value that is less than the upper quartile or the median of baseline LDL-C values among a pediatric HeFH patient cohort; adult HeFH patients) that is administered the PCSK9 inhibitor at a reference dosing frequency (e.g., an average dosing frequency for pediatric HeFH patients in the reference patient population; a standard-of-care average dosing frequency for the reference patient population; an average dosing frequency under a government regulatory agency-approved label, etc.). In some embodiments, the reduction in the pediatric subject's LDL-C is at least 70% of the reduction in LDL-C achieved in the reference patient population that is administered the PCSK9 inhibitor at the reference dosing frequency. In some embodiments, the reduction in the pediatric subject's LDL-C is substantially the same as the reduction in LDL-C achieved in the reference patient population after receiving the PCSK9 inhibitor therapy under a reference dosage regimen. In some embodiments, the enhanced dosage regimen is administered to a pediatric subject until a therapeutically acceptable end point for HeFH is achieved.

The reduction in LDL-C can be a percentage difference between the baseline LDL-C and the LDL-C after the administration of the PCSK9 inhibitor (e.g., anti-PCSK9 antibody and/or inhibitory nucleic acid). In some embodiments, the percentage reduction in LDL-C is achieved by at least week 12, at least week 13, at least week 14, at least week 15, at least week 16, at least week 17, at least week 18, at least week 19, at least week 20, at least week 21, at least week 22, at least week 23, at least week 24, at least week 25, at least week 26, at least week 27, at least week 28, at least week 29, or at least week 30 or later, or by at least a time interval within a range defined by any two of the preceding values, of administration of the PCSK9 inhibitor (e.g., anti-PCSK9 antibody and/or inhibitory nucleic acid). In some embodiments, the percentage reduction in LDL-C is achieved by at least week 20 of administration of the PCSK9 inhibitor. In some embodiments, the percentage reduction in LDL-C is achieved by at least week 24 of administration of the PCSK9 inhibitor.

Pharmaceutical compositions comprising the PCSK9 inhibitor (e.g., anti-PCSK9 antibody, inhibitory nucleic acid, or small molecular inhibitor) are administered to a subject in a manner appropriate to the indication and the composition. In some embodiments, pharmaceutical compositions comprise an anti-PCSK9 antibody. In some embodiments, pharmaceutical compositions comprise inhibitory nucleic acids (e.g., interfering RNA such as siRNA). Pharmaceutical compositions may be administered by any suitable technique, including but not limited to parenterally, topically, or by inhalation. If injected, the pharmaceutical composition can be administered, for example, via intra-articular, intravenous, intramuscular, intralesional, intraperitoneal or subcutaneous routes, by bolus injection, or continuous infusion. Delivery by inhalation includes, for example, nasal or oral inhalation, use of a nebulizer, inhalation of the antibody in aerosol form, and the like. Other alternatives include oral preparations including pills, syrups, or lozenges.

The PCSK9 inhibitor (e.g., anti-PCSK9 antibody and/or inhibitory nucleic acid) can be administered in a suitable form of a composition comprising one or more additional components such as a physiologically acceptable carrier, excipient or diluent. Optionally, the composition additionally comprises one or more physiologically active agents. In various particular embodiments, the composition comprises one, two, three, four, five, or six physiologically active agents in addition to one or more PCSK9 inhibitors (e.g., anti-PCSK9 antibodies, inhibitory nucleic acids or small molecule inhibitors).

Dosages and the frequency of administration may vary according to such factors as the route of administration, the particular PCSK9 inhibitor (e.g., anti-PCSK9 antibody, inhibitory nucleic acid, or small molecule inhibitor) employed, the nature and severity of the disease to be treated, whether the condition is acute or chronic, and the size and general condition of the subject.

### Combination Therapies

Particular embodiments of methods and compositions of the present disclosure involve the use of at least one PCSK9 inhibitor (e.g., anti-PCSK9 antibody and/or inhibitory nucleic acid) and one or more other therapeutics useful for lowering LDL-C, for example. In one embodiment, PCSK9 inhibitors (e.g., anti-PCSK9 antibodies and/or inhibitory nucleic acids) are administered alone or in combination with other agents useful for treating the condition with which the subject is afflicted. Examples of such agents include both proteinaceous and non-proteinaceous drugs. When multiple therapeutics are co-administered, dosages may be adjusted accordingly, as is recognized in the pertinent art. “Co-administration” and combination therapy are not limited to simultaneous administration, but also include treatment regimens in which an PCSK9 inhibitor is administered at least once during a course of treatment that involves administering at least one other therapeutic agent to the subject. In certain embodiments, the PCSK9 inhibitor (e.g., anti-PCSK9 antibody and/or inhibitory nucleic acid) is administered prior to the administration of at least one other therapeutic agent. In certain embodiments, a PCSK9 inhibitor (e.g., anti-PCSK9 antibody, inhibitory nucleic acid or small molecule inhibitor) is administered concurrent with the administration of at least one other therapeutic agent. In certain embodiments, a PCSK9 inhibitor (e.g., anti-PCSK9 antibody, inhibitory nucleic acid or small molecule inhibitor) is administered subsequent to the administration of at least one other therapeutic agent.

In one embodiment, the antibody and/or the inhibitory nucleic acid (e.g., interfering RNA) is administered to a subject in combination with a statin and an anti-PCSK9 antibody (e.g., Repatha® product, Praluent® product, bococizumab). In another embodiment, the antibody and/or the inhibitory nucleic acid (e.g., interfering RNA) is administered to a subject in combination with a statin and at least one other cholesterol-lowering (serum and/or total body cholesterol) agent. In some embodiments, the agents that increase the expression of LDLR, have been observed to increase serum HDL levels, lower LDL levels or lower triglyceride levels.

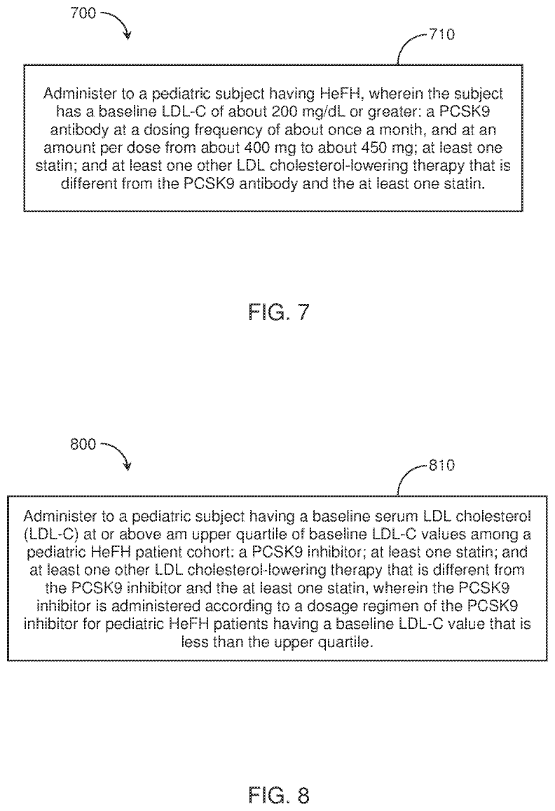

Provided herein is a method of lowering serum LDL cholesterol (LDL-C) in a subject, of which a non-limiting example is depicted in FIG. 7. The method 700 can include administering 710 to a pediatric subject having HeFH, wherein the subject has a baseline LDL-C of about 200 mg/dL or greater; a PCSK9 antibody at a dosing frequency of about once a month, and at an amount from about 400 mg to about 450 mg: at least one statin; and at least one other LDL cholesterol-lowering therapy that is different from the PCSK9 antibody and the at least one statin, to thereby lower the subject's LDL-C by at least 30%. In some embodiments, the PCSK9 antibody comprises: a heavy chain variable region (VH) comprising: a CDRH1, CDRH2, and a CDRH3 of a CDRH1, CDRH2, and a CDRH3, respectively, of a VH of evolocumab; and an amino acid sequence at least 90% identical to the VH of evolocumab; and a light chain variable region (VL) comprising: a CDRL1, CDRL2, and a CDRL3 of a CDRL1, CDRL2, and a CDRL3, respectively, of a VL of evolocumab; and an amino acid sequence at least 90% identical to the VL of evolocumab. In some embodiments, the anti-PCSK9 antibody is evolocumab.

Also provided is a method of treating or preventing heterozygous familial hypercholesterolemia (HeFH) or symptoms thereof, of which a non-limiting example is depicted in FIG. 8. The method 800 can include administering 810 to a pediatric HeFH subject having a baseline serum LDL cholesterol (LDL-C) at or above an upper quartile of baseline LDL-C values among a pediatric HeFH patient cohort, as described herein: a PCSK9 inhibitor; at least one statin; and at least one other LDL cholesterol-lowering therapy that is different from the PCSK9 inhibitor and the at least one statin, to thereby treat or prevent HeFH or symptoms thereof, wherein the PCSK9 inhibitor is administered according to a dosage regimen of the PCSK9 inhibitor for pediatric HeFH patients having a baseline LDL-C value that is less than the upper quartile, e.g., less than the median of baseline LDL-C values among the cohort.

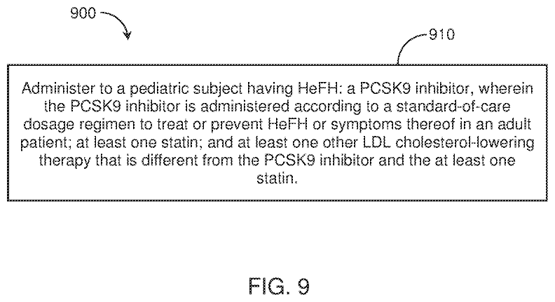

With reference to FIG. 9, a non-limiting example of a method of treating or preventing heterozygous familial hypercholesterolemia (HeFH) or symptoms thereof is described. The method 900 can include administering 910 to a pediatric subject having HeFH: a PCSK9 inhibitor, wherein the PCSK9 inhibitor is administered according to a standard-of-care (e.g., government regulatory agency-approved) dosage regimen to treat or prevent HeFH or symptoms thereof in an adult patient; at least one statin; and at least one other LDL cholesterol-lowering therapy that is different from the PCSK9 inhibitor and the at least one statin, to thereby treat or prevent HeFH or symptoms thereof.

In some embodiments, the at least one other LDL cholesterol-lowering therapy is administered according to an enhanced dosage regimen comprising a mean dose of the at least one other LDL cholesterol-lowering therapy that is about 20%, about 30%, about 40%, about 50%, about 60%, about 70%, about 80%, about 90%, about 100%, about 120%, about 140%, about 160%, about 180%, about 200%, about 250%, about 300%, about 350%, about 400%, about 450%, about 500% or more, or a percentage within a range defined by any two of the preceding values, greater than a standard-of-care (e.g., government regulatory agency-approved) mean dose of the at least one other LDL cholesterol-lowering therapy to treat or prevent HeFH or symptoms thereof in a pediatric patient.

In some embodiments, the statin includes, without limitation, atorvastatin, cerivastatin, fluvastatin, lovastatin, mevastatin, pitavastatin, pravastatin, rosuvastatin, or simvastatin. In some embodiments, the other LDL cholesterol-lowering therapy includes, without limitation, a statin, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IB AT inhibitor, a squalene synthase inhibitor, or a monocyte chemoattractant protein (MCP)-I inhibitor. Exemplary agents include, but are not limited to, statins (e.g., atorvastatin, cerivastatin, fluvastatin, lovastatin, mevastatin, pitavastatin, pravastatin, rosuvastatin, simvastatin), Nicotinic acid (Niacin) (NIACOR, NIASPAN (slow release niacin), SLO-NIACIN (slow release niacin)), Fibric acid (LOPID (Gemfibrozil), TRICOR (fenofibrate), Bile acid sequestrants (QUESTRAN (cholestyramine), colesevelam (WELCHOL), COLESTID (colestipol)), Cholesterol absorption inhibitors (ZETIA (ezetimibe)), combining nicotinic acid with statin (ADVICOR (LOVASTATIN and NIASPAN), combining a statin with an absorption inhibitor (VYTORIN (ZOCOR and ZETIA) and/or lipid modifying agents. In some embodiments, the PCSK9 inhibitor (e.g., anti-PCSK9 antibody, inhibitory nucleic acid or small molecule inhibitor) is combined with PPAR gamma agonists, PPAR alpha/gamma agonists, squalene synthase inhibitors, CETP inhibitors, anti-hypertensives, anti-diabetic agents (such as sulphonyl ureas, insulin, GLP-1 analogs, DDPIV inhibitors), ApoB modulators, MTP inhibitors and/or arteriosclerosis obliterans treatments. In some embodiments, the PCSK9 inhibitor (e.g., anti-PCSK9 antibody, inhibitory nucleic acid or small molecule inhibitor) is combined with an agent that increases the level of LDLR protein in a subject, such as statins, certain cytokines like oncostatin M, estrogen, and/or certain herbal ingredients such as berberine.

### PCSK9 Inhibitors

In some embodiments, a PCSK9 inhibitor of the present disclosure is an antibody, a inhibitory nucleic acid, or a small molecule inhibitor. In one embodiment, the anti-PCSK9 antibody is a monoclonal antibody. In one embodiment, the anti-PCSK9 antibody is a human antibody. In another embodiment, the antibodies are humanized antibodies. In another embodiment, an inhibitory nucleic acid (e.g., siRNA or shRNA) is administered in the present methods. In some embodiments, the PCSK9 inhibitor includes, without limitation, evolocumab, alirocumab, bococizumab, 1D05-IgG2, RG-7652, LGT209, REGN728, LY3015014, 1B20, inclisiran, ISIS 394814, SX-PCK9, and BMS-962476.

In some embodiments, the PCSK9 inhibitor is approved by a government regulatory agency (e.g., FDA approved) for lowering serum LDL cholesterol levels in a human patient, e.g., an adult patient.

Also contemplated herein are PCSK9 lipid lowering agents that can lower other lipids (apart from LDL-C).

### Anti-PCSK9 Antibodies

In some embodiments, the PCSK9 inhibitor is any suitable antibody that lowers LDL-C levels through PCSK9. Such PCSK9 inhibitors can include antibodies evolocumab (CAS Reg. No. 1256937-27-5; WHO No. 9643, IND No. 105188) (REPATHA®A), alirocumab (PRALUENT®), bococizumab, REGN728, RG7652, LY3015014, LGT209, 1D05 (U.S. Pat. No. 8,188,234), 1B20 (U.S. Pat. No. 8,188,233), SX-PCK9 and BMS-962476. In some embodiments, the antibody is a neutralizing antibody. For conciseness, an “anti-PCSK9 antibody” may also be referred to herein as a “PCSK9 antibody,” and it will be understood that these two terms are interchangeable herein.

In some embodiments, the PCSK9 antibody comprises: a heavy chain variable region (VH) comprising a CDRH1, CDRH2, and a CDRH3 of a CDRH1, CDRH2, and a CDRH3, respectively, of a heavy chain of evolocumab; and a light chain variable region (VL) comprising a CDRL1, CDRL2, and a CDRL3 of a CDRL1, CDRL2, and a CDRL3, respectively, of a light chain of evolocumab, as shown in FIG. 14. In some embodiments, the PCSK9 antibody comprises: a heavy chain variable region (VH) comprising a CDRH1, CDRH2, and a CDRH3 of a CDRH1, CDRH2, and a CDRH3, respectively, of a heavy chain of evolocumab with up to 3, 2, or 1 amino acid substitutions (for example, conservative substitutions) in one or more of the CDRH1, CDRH2, and CDRH3, respectively; and/or a light chain variable region (VL) comprising a CDRL1, CDRL2, and a CDRL3 of a CDRL1, CDRL2, and a CDRL3, respectively, of a light chain of evolocumab with up to 3, 2, or 1 amino acid substitutions (for example, conservative substitutions) in one or more of the CDRL1, CDRL2, and CDRL3, respectively. In some embodiments, the PCSK9 antibody comprises: a heavy chain variable region (VH) comprising a CDRH1, CDRH2, and a CDRH3 of a CDRH1, CDRH2, and a CDRH3, respectively, of a heavy chain of evolocumab with up to 3, 2, or 1 amino acid substitutions (for example, conservative substitutions) in each of the CDRH1, CDRH2, and CDRH3, respectively; and a light chain variable region (VL) comprising a CDRL1, CDRL2, and a CDRL3 of a CDRL1, CDRL2, and a CDRL3, respectively, of a light chain of evolocumab with up to 3 amino acid substitutions (for example, conservative substitutions) in each of the CDRL1, CDRL2, and CDRL3, respectively. In some embodiments, the PCSK9 antibody comprises: a heavy chain variable region (VH) comprising a CDRH1, CDRH2, and a CDRH3 that is each independently at least 80, 85, 90, 95%, or 100% identical to a CDRH1, CDRH2, and a CDRH3, respectively, of a heavy chain of evolocumab; and a light chain variable region (VL) comprising a CDRL1, CDRL2, and a CDRL3 that is each independently at least 80, 85, 90, 95%, or 100% identical to a CDRL1, CDRL2, and a CDRL3, respectively, of a light chain of evolocumab, as shown in FIG. 14. In some embodiments, the PCSK9 antibody comprises: a heavy chain variable region (VH) comprising a framework region (FR) 1, FR2, FR3 and a FR4 of a FR1, FR2, FR3 and a FR4, respectively, of a heavy chain of evolocumab with up to 3, 2, or 1 amino acid substitutions (for example, conservative substitutions) in one or more of the FR1, FR2, FR3 and FR4, respectively; and/or a light chain variable region (VL) comprising a FR1, FR2, FR3 and a FR4 of a FR1, FR2, FR3 and a FR4, respectively, of a light chain of evolocumab with up to 3, 2, 1 amino acid substitutions (for example, conservative substitutions) in one or more of the FR1, FR2, FR3 and FR4, respectively. In some embodiments, the PCSK9 antibody comprises: a heavy chain variable region (VH) comprising a FR1, FR2, FR3 and a FR4 that is each independently at least 80, 85, 90, 95%, or 100% identical to a FR1, FR2, FR3 and a FR4, respectively, of a heavy chain of evolocumab; and a light chain variable region (VL) comprising a FR1, FR2, FR3 and a FR4 that is each independently at least 80, 85, 90, 95%, or 1000% identical to a FR1, FR2, FR3 and a FR4, respectively, of a light chain of evolocumab, as shown in FIG. 14. In some embodiments, the PCSK9 antibody comprises: a VH comprising an amino acid sequence at least 85%, at least 90%, at least 95%, or at least 98% or more identical to the VH of evolocumab; and a VL comprising an amino acid sequence at least 85%, at least 90%, at least 95%, or at least 98% or more identical to the VL of evolocumab, as shown in FIG. 14. In some embodiments, the PCSK9 antibody comprises: a VH comprising: a CDRH1, CDRH2, and a CDRH3 of a CDRH1, CDRH2, and a CDRH3, respectively, of a VH of evolocumab; and an amino acid sequence at least 85%, at least 90%, at least 95%, or at least 98% or more identical to the VH of evolocumab; and a VL comprising: a CDRL1, CDRL2, and a CDRL3 of a CDRL1, CDRL2, and a CDRL3, respectively, of a VL of evolocumab; and an amino acid sequence at least 85%, at least 90%, at least 95%, or at least 98% or more identical to the VL of evolocumab. In some embodiments, the PCSK9 antibody comprises a VH of a VH of evolocumab; and VL of a VL of evolocumab. In some embodiments, the PCSK9 antibody comprises: a heavy chain variable region (VH) comprising: a CDRH1, CDRH2, and a CDRH3 of a CDRH1, CDRH2, and a CDRH3, respectively, of a VH of evolocumab; and an amino acid sequence at least 90% identical to the VH of evolocumab; and a light chain variable region (VL) comprising: a CDRL1, CDRL2, and a CDRL3 of a CDRL1, CDRL2, and a CDRL3, respectively, of a VL of evolocumab; and an amino acid sequence at least 90% identical to the VL of evolocumab. In some embodiments, the anti-PCSK9 antibody is evolocumab.

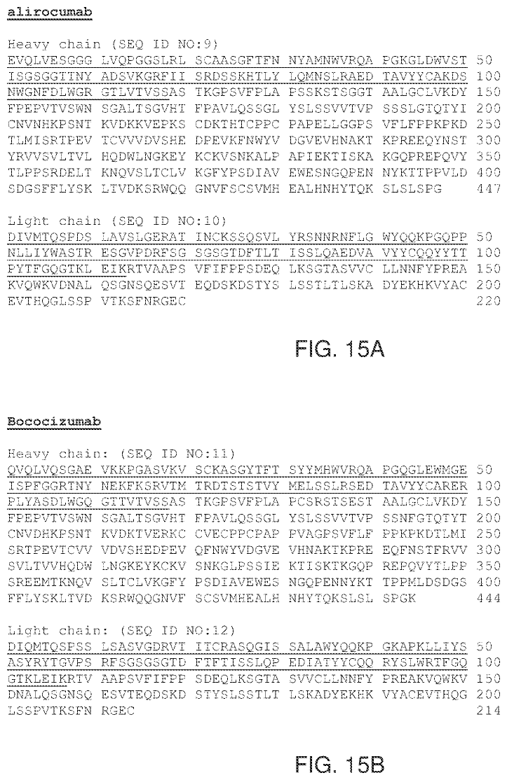

In some embodiments, the PCSK9 antibody comprises: a heavy chain variable region (VH) comprising a CDRH1, CDRH2, and a CDRH3 of a CDRH1, CDRH2, and a CDRH3, respectively, of a heavy chain of alirocumab; and a light chain variable region (VL) comprising a CDRL1, CDRL2, and a CDRL3 of a CDRL1, CDRL2, and a CDRL3, respectively, of a light chain of alirocumab, as shown in FIG. 15A. In some embodiments, the PCSK9 antibody comprises: a heavy chain variable region (VH) comprising a CDRH1, CDRH2, and a CDRH3 of a CDRH1, CDRH2, and a CDRH3, respectively, of a heavy chain of alirocumab with up to 3, 2, or 1 amino acid substitutions (for example, conservative substitutions) in one or more of the CDRH1, CDRH2, and CDRH3, respectively; and/or a light chain variable region (VL) comprising a CDRL1, CDRL2, and a CDRL3 of a CDRL1, CDRL2, and a CDRL3, respectively, of a light chain of alirocumab with up to 3, 2, or 1 amino acid substitutions (for example, conservative substitutions) in one or more of the CDRL1, CDRL2, and CDRL3, respectively. In some embodiments, the PCSK9 antibody comprises: a heavy chain variable region (VH) comprising a CDRH1, CDRH2, and a CDRH3 that is each independently at least 80, 85, 90, 95%, or 100% identical to a CDRH1, CDRH2, and a CDRH3, respectively, of a heavy chain of alirocumab; and a light chain variable region (VL) comprising a CDRL1, CDRL2, and a CDRL3 that is each independently at least 80, 85, 90, 95%, or 100% identical to a CDRL1, CDRL2, and a CDRL3, respectively, of a light chain of alirocumab, as shown in FIG. 15A. In some embodiments, the PCSK9 antibody comprises: a heavy chain variable region (VH) comprising a FR1, FR2, FR3 and a FR4 of a FR1, FR2, FR3 and a FR4, respectively, of a heavy chain of alirocumab with up to 3, 2, or 1 amino acid substitutions (for example, conservative substitutions) in one or more of the FR1, FR2, FR3 and FR4, respectively; and/or a light chain variable region (VL) comprising a FR1, FR2, FR3 and a FR4 of a FR1, FR2, FR3 and a FR4, respectively, of a light chain of alirocumab with up to 3, 2, or 1 amino acid substitutions (for example, conservative substitutions) in one or more of the FR1, FR2, FR3 and FR4, respectively. In some embodiments, the PCSK9 antibody comprises: a heavy chain variable region (VH) comprising a FR1, FR2, FR3 and a FR4 that is each independently at least 80, 85, 90, 95%, or 100% identical to a FR1, FR2, FR3 and a FR4, respectively, of a heavy chain of alirocumab; and a light chain variable region (VL) comprising a FR1, FR2, FR3 and a FR4 that is each independently at least 80, 85, 90, 95%, or 100% identical to a FR1, FR2, FR3 and a FR4, respectively, of a light chain of alirocumab, as shown in FIG. 15A. In some embodiments, the PCSK9 antibody comprises: a VH comprising an amino acid sequence at least 85%, at least 90%, at least 95%, or at least 98% or more identical to the VH of alirocumab; and a VL comprising an amino acid sequence at least 85%, at least 90%, at least 95%, or at least 98% or more identical to the VL of alirocumab. In some embodiments, the PCSK9 antibody comprises: a VH comprising; a CDRH1, CDRH2, and a CDRH3 of a CDRH1, CDRH2, and a CDRH3, respectively, of a VH of alirocumab; and an amino acid sequence at least 85%, at least 90%, at least 95%, or at least 98% or more identical to the VH of alirocumab; and a VL comprising: a CDRL1, CDRL2, and a CDRL3 of a CDRL1, CDRL2, and a CDRL3, respectively, of a VL of alirocumab; and an amino acid sequence at least 85%, at least 90%, at least 95%, or at least 98% or more identical to the VL of alirocumab. In some embodiments, the anti-PCSK9 antibody is alirocumab.

In some embodiments, the PCSK9 antibody comprises: a heavy chain variable region (VH) comprising a CDRH1, CDRH2, and a CDRH3 of a CDRH1, CDRH2, and a CDRH3, respectively, of a heavy chain of bococizumab; and a light chain variable region (VL) comprising a CDRL1, CDRL2, and a CDRL3 of a CDRL1, CDRL2, and a CDRL3, respectively, of a light chain of bococizumab, as shown in FIG. 15B. In some embodiments, the PCSK9 antibody comprises: a heavy chain variable region (VH) comprising a CDRH1, CDRH2, and a CDRH3 of a CDRH1, CDRH2, and a CDRH3, respectively, of a heavy chain of bococizumab with up to 3, 2, or 1 amino acid substitutions (for example, conservative substitutions) in one or more of the CDRH1, CDRH2, and CDRH3, respectively; and/or a light chain variable region (VL) comprising a CDRL1, CDRL2, and a CDRL3 of a CDRL1, CDRL2, and a CDRL3, respectively, of a light chain of bococizumab with up to 3, 2, or 1 amino acid substitutions (for example, conservative substitutions) in one or more of the CDRL1, CDRL2, and CDRL3. In some embodiments, the PCSK9 antibody comprises: a heavy chain variable region (VH) comprising a CDRH1, CDRH2, and a CDRH3 that is each independently at least 80, 85, 90, 95%, or 100% identical to a CDRH1, CDRH2, and a CDRH3, respectively, of a heavy chain of bococizumab; and a light chain variable region (VL) comprising a CDRL1, CDRL2, and a CDRL3 that is each independently at least 80, 85, 90, 95%, or 100% identical to a CDRL1, CDRL2, and a CDRL3, respectively, of a light chain of bococizumab, as shown in FIG. 15B. In some embodiments, the PCSK9 antibody comprises: a heavy chain variable region (VH) comprising a FR1, FR2, FR3 and a FR4 of a FR1, FR2, FR3 and a FR4, respectively, of a heavy chain of bococizumab with up to 3, 2, or 1 amino acid substitutions (for example, conservative substitutions) in one or more of the FR1, FR2, FR3 and FR4, respectively; and/or a light chain variable region (VL) comprising a FR1, FR2, FR3 and a FR4 of a FR1, FR2, FR3 and a FR4, respectively, of a light chain of bococizumab with up to 3, 2, or 1 amino acid substitutions (for example, conservative substitutions) in one or more of the FR1, FR2, FR3 and FR4, respectively. In some embodiments, the PCSK9 antibody comprises: a heavy chain variable region (VH) comprising a FR1, FR2, FR3 and a FR4 that is each independently at least 80, 85, 90, 95%, or 100% identical to a FR1, FR2, FR3 and a FR4, respectively, of a heavy chain of bococizumab; and a light chain variable region (VL) comprising a FR1, FR2, FR3 and a FR4 that is each independently at least 80, 85, 90, 95%, or 100%/c identical to a FR1, FR2, FR3 and a FR4, respectively, of a light chain of bococizumab, as shown in FIG. 15B. In some embodiments, the PCSK9 antibody comprises an amino acid sequence at least 85%, at least 90%, at least 95%, or at least 98% or more identical to the VH of bococizumab; and a VL comprising an amino acid sequence at least 85%, at least 90%, at least 95%, or at least 98% or more identical to the VL of bococizumab. In some embodiments, the PCSK9 antibody comprises: a VH comprising: a CDRH1, CDRH2, and a CDRH3 of a CDRH1, CDRH2, and a CDRH3, respectively, of a VH of bococizumab; and an amino acid sequence at least 85%, at least 90%, at least 95%, or at least 98% or more identical to the VH of bococizumab; and a VL comprising: a CDRL1, CDRL2, and a CDRL3 of a CDRL1, CDRL2, and a CDRL3, respectively, of a VL of bococizumab; and an amino acid sequence at least 85%, at least 90%, at least 95%, or at least 98% or more identical to the VL of bococizumab. In some embodiments, the anti-PCSK9 antibody is bococizumab.

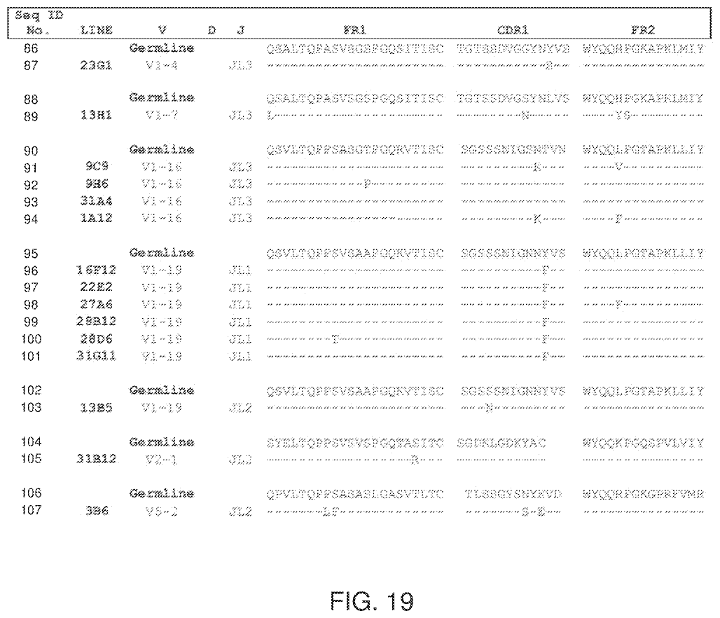

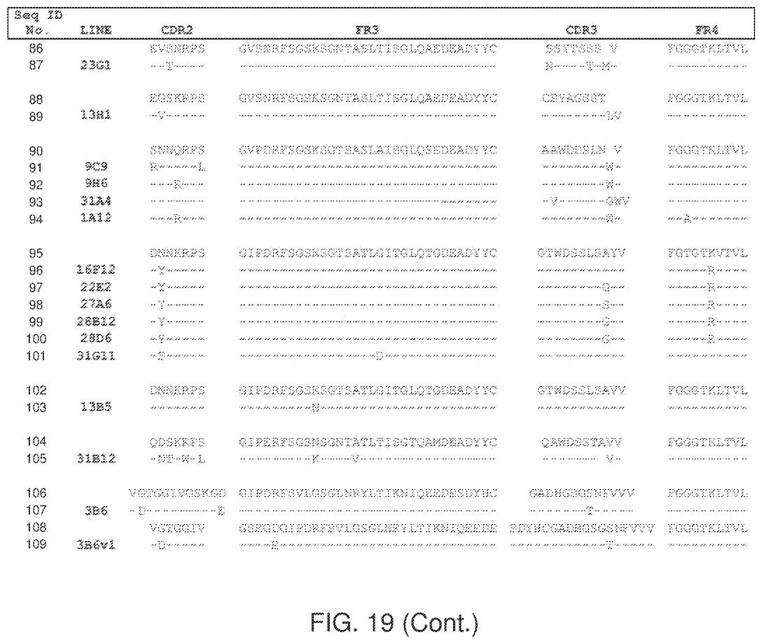

In some embodiments, the inhibitor is an anti-PCSK9 antibody that contains one or more (including all 6) of the CDRs from the antibody constructs shown in any one or more of FIGS. 14, 15A, 15B, 16, 17, 18 and 19. In some embodiments, the PCSK9 inhibitor is an anti-PCSK9 antibody that contains one or more of the amino acid heavy and/or light chains of FIGS. 14, 15A, 15B, 16, 17, 18 and 19. In some embodiments, the PCSK9 inhibitor is an anti-PCSK9 antibody that contains one or more of the amino acid heavy chains, variable regions, and/or CDRs of FIGS. 14, 15A, 15B, 16, 17, and the corresponding amino acid light chains, variable regions, and/or CDRs of FIGS. 14, 15A, 15B, 18 and 19. In some embodiments, antibodies that include any one or more of the CDRs of the antibodies noted herein can be employed. In some embodiments, antibodies that include the heavy and light chain variable regions of the antibodies noted herein can be employed. In some embodiments, the antibody is at least 95, 96, 97, 98, 99% identical in amino acid sequence to an antibody denoted herein. In some embodiments, the anti-PCSK9 antibody is selected from the antibodies in U.S. Pat. No. 8,062,640 (e.g., HCVR/LCVR=SEQ ID NOS:90/92), U.S. Pat. No. 8,501,184 (e.g., REGN728, HCVR/LCVR=SEQ ID NOS:218/226), U.S. Pat. No. 8,080,243 (e.g., bococizumab, HCVR/LCVR=SEQ ID Nos:54/53), U.S. Pat. No. 8,188,234 (e.g., 1D05, HCVR/LCVR=SEQ ID Nos:11/27), U.S. Pat. No. 8,188,233 (e.g., 1B20, HCVR/LCVR=SEQ ID Nos:11/27), LGT209 in U.S. Pat. No. 8,710,192, US2011/0142849, and US2013/0315927, and RG7652 in US2012/0195910, LY3015014 in U.S. Pat. No. 8,530,414 (HCVR/LCVR=SEQ ID Nos:7/8), the entireties of each of which is hereby incorporated by reference including the disclosure of the specifically referenced PCSK9 inhibitors.

In some embodiments, the anti-PCSK9 antibody includes up to 1, 2, 3, 4 or 5 amino acid mutations to one or more of the CDRs (including the HCDR1, HCDR2, HCDR3, LCDR1, LCDR2, and/or LCDR3) and/or any of the framework regions of any of the light chains or heavy chains of FIGS. 14, 15A, 15B, 16, 17, 18 and 19. In some embodiments, the anti-PCSK9 antibody has any of the light chains or heavy chains of FIGS. 14, 15A, 15B, 16, 17, 18 and 19, but where any of the CDRs (including the HCDR1, HCDR2, HCDR3, LCDR1, LCDR2, and/or LCDR3) are variants of the disclosed sequences, such that the CDR(s) is, each independently, at least 80, 85, or 90% identical to the corresponding sequence provided herein. In some embodiments, any mutated position is a conservative substitution. In some embodiments, the conservative mutation is one or more of the options put forth in Table 1.0.

In some embodiments, the anti-PCSK9 antibody includes up to 3, 2, or 1 mutations in one or more CDRs (including the HCDR1, HCDR2, HCDR3, LCDR1, LCDR2, and/or LCDR3) and/or one or more framework regions relative to an amino acid sequence encoded by the variable region of the human germline immunoglobulin sequence. In some embodiments, the anti-PCSK9 antibody includes CDRs (including the HCDR1, HCDR2, HCDR3, LCDR1, LCDR2, and/or LCDR3) that are each independently at least 80%, 85%, 90%, 95%, 98% or more identical to a corresponding CDR encoded by a human germline immunoglobulin sequence. In some embodiments, the anti-PCSK9 antibody includes light chain FRs (including the FR1, FR2, FR3, and/or FR4) and/or heavy chain FRs (including the FR1, FR2, FR3, and/or FR4) that are each independently at least 80%, 85%, 90%, 95%, 98%, 99%, or about 100% identical to a corresponding FR encoded by a human germline immunoglobulin sequence. In some embodiments, the anti-PCSK9 antibody includes: (i) a heavy chain variable region encoded by human germline VH1-18 and JH6B, with up to 3, 2, or 1 mutations in one or more CDRs (including the HCDR1, HCDR2, and/or HCDR3) and/or one or more framework regions relative to an amino acid sequence encoded by the human germline VH1-18 and JH6B: and (ii) a light chain variable region encoded by human germline V1-4 and JL2, with up to 3, 2, or 1 mutations in one or more CDRs (including the LCDR1, LCDR2, and/or LCDR3) and/or one or more framework regions relative to an amino acid sequence encoded by the human germline V1-4 and JL2. In some embodiments, the anti-PCSK9 antibody includes a heavy chain variable region encoded by human germline VH1-18 and JH6B, with up to 3, 2, or 1 mutations in one or more CDRs (including the HCDR1, HCDR2, and/or HCDR3) and/or one or more framework regions relative to an amino acid sequence encoded by the human germline VH1-18 and JH6B. In some embodiments, the anti-PCSK9 antibody includes a heavy chain variable region encoded by human germline VH1-18 and JH16B, where the CDRs (including the HCDR1, HCDR2, and/or HCDR3) are each independently at least 80%, 85%, 90%, 95%, 98% or more identical to a corresponding CDR encoded by human germline VH1-18 and JH6B. In some embodiments, the anti-PCSK9 antibody includes a heavy chain variable region encoded by human germline VH1-18 and JH16B, where the FRs (including the FR1, FR2, FR3, and/or FR4) are each independently at least 80%, 85%, 90%, 95%, 98% or more identical to a corresponding FR encoded by human germline VH1-18 and JH6B. In some embodiments, the anti-PCSK9 antibody includes a light chain variable region encoded by human germline V1-4 and JL2, with up to 3, 2, or 1 mutations in one or more CDRs (including the LCDR1, LCDR2, and/or LCDR3) and/or one or more framework regions relative to an amino acid sequence encoded by the human germline V1-4 and JL2. In some embodiments, the anti-PCSK9 antibody includes a light chain variable region encoded by human germline V1-4 and JL2, where the CDRs (including the LCDR1, LCDR2, and/or LCDR3) are each independently at least 80%, 85%, 90%, 95%, 98% or more identical to a corresponding CDR encoded by human germline V1-4 and JL2. In some embodiments, the anti-PCSK9 antibody includes a light chain variable region encoded by human germline V1-4 and JL2, where the FRs (including the FR1, FR2, FR3, and/or FR4) are each independently at least 80%, 85%, 90%, 95%, 98% or more identical to a corresponding FR encoded by human germline V1-4 and JL2. In some embodiments, any mutation is a conservative substitution. In some embodiments, the conservative mutation is one or more of the options put forth in table 1.0.

In some embodiments, mutations in the CDRs and/or framework regions preserve residues that form an interaction interface with a bound PCSK9 protein of the original antibody. Anti-PCSK9 antibody variable region residues that form an interaction interface with a bound PCSK9 protein are known and disclosed in, for example, Example 30 of U.S. Patent Application Publication No. 2009/0142352, which is incorporated herein by reference in its entirety.

The anti-PCSK9 antibodies of the present disclosure can comprise any suitable constant region known in the art. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region.

Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See also Lanitto et al., Methods Mol. Biol. 178:303-16 (2002).

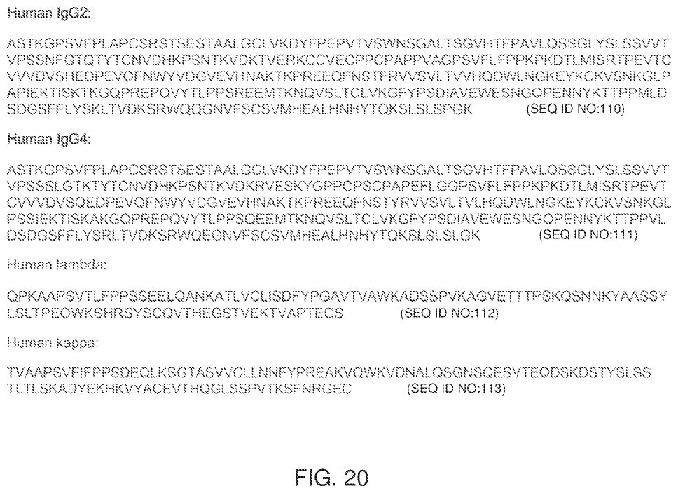

In one embodiment, an anti-PCSK9 antibody of the present disclosure further comprises the constant light chain kappa or lambda domains or a fragment of these. Exemplary sequences of the light chain constant regions are provided in FIG. 20, and are generally well known in the art. In another embodiment, an anti-PCSK9 antibody of the present disclosure further comprises a heavy chain constant domain, or a fragment thereof, such as the IgG1 or IgG2 heavy chain constant region. In another embodiment, an anti-PCSK9 antibody of the present disclosure further comprises a heavy chain constant domain, or a fragment thereof, such as the IgG2 or IgG4 heavy chain constant regions, examples of amino acid sequences of which are provided in FIG. 20.

The anti-PCSK9 antibodies of the present disclosure include those having a desired isotype (for example, IgA, IgG1, IgG2, IgG3, IgG4, IgM, IgE, and IgD) as well as Fab or F(ab′)₂ fragments thereof. Moreover, if an IgG4 is desired, it may also be desired to introduce a point mutation in the hinge region as described in Bloom et al., 1997, Protein Science 6:407, (incorporated by reference herein) to alleviate a tendency to form intra-H chain disulfide bonds that can lead to heterogeneity in the IgG4 antibodies.

### Generation of Antibodies

Antibodies of the present disclosure may be prepared by techniques that are well known to those skilled in the art. For example, by immunizing an animal (e.g., a mouse or rat or rabbit) and then by immortalizing spleen cells harvested from the animal after completion of the immunization schedule. The spleen cells can be immortalized using any technique known in the art, e.g., by fusing them with myeloma cells to produce hybridomas. See, for example, Antibodies; Harlow and Lane, Cold Spring Harbor Laboratory Press, 1^{st} Edition, e.g. from 1988, or 2^{nd} Edition, e.g. from 2014).

In one embodiment, a humanized monoclonal antibody comprises the variable domain of a murine antibody (or all or part of the antigen binding site thereof) and a constant domain derived from a human antibody. Alternatively, a humanized antibody fragment may comprise the antigen binding site of a murine monoclonal antibody and a variable domain fragment (lacking the antigen-binding site) derived from a human antibody. Procedures for the production of engineered monoclonal antibodies include those described in Riechmann et al., 1988, Nature 332:323, Liu et al., 1987, Proc. Nat. Acad. Sci. USA 84:3439, Larrick et al., 1989, Bio/Technology 7:934, and Winter et al., 1993, TIPS 14:139. In one embodiment, the chimeric antibody is a CDR grafted antibody. Techniques for humanizing antibodies are discussed in, e.g., U.S. Pat. Nos. 5,869,619; 5,225,539; 5,821,337; 5,859,205; 6,881,557, Padlan et al., 1995, FASEB J. 9:133-39, Tamura et al., 2000, J. Immunol. 164:1432-41, Zhang, W., et al., Molecular Immunology. 42(12):1445-1451, 2005; Hwang W. et al., Methods. 36(1):35-42, 2005: Dall'Acqua W F, et al., Methods 36(1):43-60, 2005: and Clark, M., Immunology Today. 21(8):397-402, 2000.

An antibody of the present disclosure may also be a fully human monoclonal antibody. Fully human monoclonal antibodies may be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B-cells, fusion of spleen cells from immunized transgenic mice carrying inserted human immunoglobulin genes, isolation from human immunoglobulin V region phage libraries, or other procedures as known in the art and based on the disclosure herein.

Procedures have been developed for generating human monoclonal antibodies in non-human animals. For example, mice in which one or more endogenous immunoglobulin genes have been inactivated by various means have been prepared. Human immunoglobulin genes have been introduced into the mice to replace the inactivated mouse genes. In this technique, elements of the human heavy and light chain locus are introduced into strains of mice derived from embryonic stem cell lines that contain targeted disruptions of the endogenous heavy chain and light chain loci (see also Bruggemann et al., Curr. Opin. Biotechnol. 8:455-58 (1997)). For example, human immunoglobulin transgenes may be mini-gene constructs, or transloci on yeast artificial chromosomes, which undergo B-cell-specific DNA rearrangement and hypermutation in the mouse lymphoid tissue.

Antibodies produced in the animal incorporate human immunoglobulin polypeptide chains encoded by the human genetic material introduced into the animal. In one embodiment, a non-human animal, such as a transgenic mouse, is immunized with a suitable immunogen.

Examples of techniques for production and use of transgenic animals for the production of human or partially human antibodies are described in U.S. Pat. Nos. 5,814,318, 5,569,825, and 5,545,806, Davis et al., Production of human antibodies from transgenic mice in Lo, ed. Antibody Engineering: Methods and Protocols, Humana Press, NJ: 191-200 (2003), Kellermann et al., 2002, Curr Opin Biotechnol. 13:593-97, Russel et al., 2000, Infect Immun. 68:1820-26, Gallo et al., 2000, Eur J Immun. 30:534-40, Davis et al., 1999, Cancer Metastasis Rev. 18:421-25, Green, 1999, J Immunol Methods. 231:11-23, Jakobovits, 1998, Advanced Drug Delivery Reviews 31:33-42, Green et al., 1998, J Exp Med. 188:483-95, Jakobovits A, 1998, Exp. Opin. Invest. Drugs. 7:607-14, Tsuda et al., 1997, Genomics. 42:413-21, Mendez et al., 1997, Nat Genet. 15:146-56, Jakobovits, 1994, Curr Biol. 4:761-63, Arbones et al., 1994, Immunity. 1:247-60, Green et al., 1994, Nat Genet. 7:13-21, Jakobovits et al., 1993, Nature. 362:255-58, Jakobovits et al., 1993, Proc Natl Acad Sci USA. 90:2551-55. Chen, J., M. Trounstine, F. W. Alt, F. Young, C. Kurahara, J. Loring, D. Huszar. “Immunoglobulin gene rearrangement in B-cell deficient mice generated by targeted deletion of the JH locus.” International Immunology 5 (1993): 647-656, Choi et al., 1993, Nature Genetics 4: 117-23, Fishwild et al., 1996, Nature Biotechnology 14: 845-51, Harding et al., 1995, Annals of the New York Academy of Sciences, Lonberg et al., 1994, Nature 368: 856-59, Lonberg, 1994, Transgenic Approaches to Human Monoclonal Antibodies in Handbook of Experimental Pharmacology 113: 49-101, Lonberg et al., 1995, Internal Review of Immunology 13: 65-93, Neuberger, 1996, Nature Biotechnology 14: 826, Taylor et al., 1992, Nucleic Acids Research 20: 6287-95, Taylor et al., 1994, International Immunology 6: 579-91, Tomizuka et al., 1997, Nature Genetics 16: 133-43, Tomizuka et al., 2000, Proceedings of the National Academy of Sciences USA 97: 722-27, Tuaillon et al., 1993, Proceedings of the National Academy of Sciences USA 90: 3720-24, and Tuaillon et al., 1994, Journal of Immunology 152: 2912-20; Lonberg et al., Nature 368:856, 1994; Taylor et al., Int. Immun. 6:579, 1994; U.S. Pat. No. 5,877,397; Bruggemann et al., 1997 Curr. Opin. Biotechnol. 8:455-58; Jakobovits et al., 1995 Ann. N. Y. Acad. Sci. 764:525-35. In addition, protocols involving the XenoMouse® (Abgenix, now Amgen, Inc.) are described, for example in U.S. Ser. No. 05/011,8643 and WO 05/694879, WO 98/24838, WO 00/76310, and U.S. Pat. No. 7,064,244.

Lymphoid cells from the immunized transgenic mice are fused with myeloma cells for example to produce hybridomas. Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in such fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; examples of cell lines used in rat fusions include R210.RCY3, Y3-Ag 1.2.3, IR983F and 4B210. Other cell lines useful for cell fusions are U-266, GM1500-GRG2, LCR-LON-HMy2 and UC729-6.

The lymphoid (e.g., spleen) cells and the myeloma cells may be combined for a few minutes with a membrane fusion-promoting agent, such as polyethylene glycol or a nonionic detergent, and then plated at low density on a selective medium that supports the growth of hybridoma cells but not unfused myeloma cells. One selection media is HAT (hypoxanthine, aminopterin, thymidine). After a sufficient time, usually about one to two weeks, colonies of cells are observed. Single colonies are isolated, and antibodies produced by the cells may be tested for binding activity to, for example, human PCSK9, using any one of a variety of immunoassays known in the art and described herein. The hybridomas are cloned (e.g., by limited dilution cloning or by soft agar plaque isolation) and positive clones that produce an antibody specific to, for example, human PCSK9, are selected and cultured. The monoclonal antibodies from the hybridoma cultures may be isolated from the supernatants of hybridoma cultures. Thus the present disclosure provides hybridomas that comprise polynucleotides encoding the PCSK9 inhibitors of the present disclosure in the chromosomes of the cell. These hybridomas can be cultured according to methods described herein and known in the art.

Another method for generating human antibodies of the present disclosure includes immortalizing human peripheral blood cells by EBV transformation. See, e.g., U.S. Pat. No. 4,464,456. Such an immortalized B-cell line (or lymphoblastoid cell line) producing a monoclonal antibody that specifically binds to, for example, human PCSK9, can be identified by immunodetection methods as provided herein, for example, an ELISA, and then isolated by standard cloning techniques. The stability of the lymphoblastoid cell line producing an antibody may be improved by fusing the transformed cell line with a murine myeloma to produce a mouse-human hybrid cell line according to methods known in the art (see, e.g., Glasky et al., Hybridoma 8:377-89 (1989)). Still another method to generate human monoclonal antibodies is in vitro immunization, which includes priming human splenic B-cells with antigen, followed by fusion of primed B-cells with a heterohybrid fusion partner. See, e.g., Boerner et al., 1991 J. Immunol. 147:86-95.

In certain embodiments, a B-cell that is producing a desired antibody is selected and the light chain and heavy chain variable regions are cloned from the B-cell according to molecular biology techniques known in the art (WO 92/02551; U.S. Pat. No. 5,627,052; Babcook et al., Proc. Natl. Acad. Sci. USA 93:7843-48 (1996)) and described herein. B-cells from an immunized animal may be isolated from the spleen, lymph node, or peripheral blood sample by selecting a cell that is producing a desired antibody. B-cells may also be isolated from humans, for example, from a peripheral blood sample. Methods for detecting single B-cells that are producing an antibody with the desired specificity are well known in the art, for example, by plaque formation, fluorescence-activated cell sorting, in vitro stimulation followed by detection of specific antibody, and the like. Methods for selection of specific antibody-producing B-cells include, for example, preparing a single cell suspension of B-cells in soft agar that contains antigen. Binding of the specific antibody produced by the B-cell to the antigen results in the formation of a complex, which may be visible as an immunoprecipitate. After the B-cells producing the desired antibody are selected, the specific antibody genes may be cloned by isolating and amplifying DNA or mRNA according to methods known in the art and described herein.

An additional method for obtaining antibodies of the present disclosure is by phage display. See, e.g., Winter et al., 1994 Annu. Rev. Immunol. 12:433-55; Burton et al., 1994 Adv. Immunol. 57:191-280. Human or murine immunoglobulin variable region gene combinatorial libraries may be created in phage vectors that can be screened to select Ig fragments (Fab, Fv, sFv, or multimers thereof) that bind specifically to PCSK9 or variant or fragment thereof. See, e.g., U.S. Pat. No. 5,223,409; Huse et al., 1989 Science 246:1275-81; Sastry et al., Proc. Natl. Acad. Sci. USA 86:5728-32 (1989); Alting-Mees et al., Strategies in Molecular Biology 3:1-9 (1990); Kang et al., 1991 Proc. Natl. Acad. Sci. USA 88:4363-66; Hoogenboom et al., 1992 J. Molec. Biol. 227:381-388; Schlebusch et al., 1997 Hybridoma 16:47-52 and references cited therein. For example, a library containing a plurality of polynucleotide sequences encoding Ig variable region fragments may be inserted into the genome of a filamentous bacteriophage, such as M13 or a variant thereof, in frame with the sequence encoding a phage coat protein. A fusion protein may be a fusion of the coat protein with the light chain variable region domain and/or with the heavy chain variable region domain. According to certain embodiments, immunoglobulin Fab fragments may also be displayed on a phage particle (see, e.g., U.S. Pat. No. 5,698,426).

Heavy and light chain immunoglobulin cDNA expression libraries may also be prepared in lambda phage, for example, using λImmunoZap™(H) and λImmunoZap™(L) vectors (Stratagene, La Jolla, Calif.). Briefly, mRNA is isolated from a B-cell population, and used to create heavy and light chain immunoglobulin cDNA expression libraries in the λImmunoZap(H) and λImmunoZap(L) vectors. These vectors may be screened individually or co-expressed to form Fab fragments or antibodies (see Huse et al., supra; see also Sastry et al., supra). Positive plaques may subsequently be converted to a non-lytic plasmid that allows high level expression of monoclonal antibody fragments from E. coli.

In one embodiment, in a hybridoma the variable regions of a gene expressing a monoclonal antibody of interest are amplified using nucleotide primers. These primers may be synthesized by one of ordinary skill in the art, or may be purchased from commercially available sources. (See, e.g., Stratagene (La Jolla, Calif.), which sells primers for mouse and human variable regions including, among others, primers for V_{Ha}, V_{Hb}, V_{Hc}, V_{Hd}, C_{H1}, V_{L} and C_{L} regions.) These primers may be used to amplify heavy or light chain variable regions, which may then be inserted into vectors such as ImmunoZAP™H or ImmunoZAP™L (Stratagene), respectively. These vectors may then be introduced into E. coli, yeast, or mammalian-based systems for expression. Large amounts of a single-chain protein containing a fusion of the V_{H} and V_{L} domains may be produced using these methods (see Bird et al., Science 242:423-426, 1988).

In certain embodiments, the PCSK9 inhibitors (e.g., anti-PCKS9 antibodies) of the present disclosure are obtained from transgenic animals (e.g., mice) that produce “heavy chain only” antibodies or “HCAbs.” HCAbs are analogous to naturally occurring camel and llama single-chain VHH antibodies.

See, for example, U.S. Pat. Nos. 8,507,748 and 8,502,014, and U.S. Patent Application Publication Nos. US2009/0285805A1, US2009/0169548A1, US2009/0307787A1, US2011/0314563A1, US2012′0151610A1, WO2008/122886A2, and WO2009/013620A2.

Once cells producing antibodies according to the present disclosure have been obtained using any of the above-described immunization and other techniques, the specific antibody genes may be cloned by isolating and amplifying DNA or mRNA therefrom according to standard procedures as described herein. The antibodies produced therefrom may be sequenced and the CDRs identified and the DNA coding for the CDRs may be manipulated as described previously to generate other antibodies according to the present disclosure.

In certain embodiments, antibodies are generated by first identifying antibodies that bind to cells expressing, for example, human PCSK9 and/or compete for binding with the antibodies described in this application.

It will be understood by one skilled in the art that some proteins, such as antibodies, may undergo a variety of posttranslational modifications. The type and extent of these modifications often depends on the host cell line used to express the protein as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, R. J. Journal of Chromatography 705:129-134, 1995).

An alternative method for production of a murine monoclonal antibody is to inject the hybridoma cells into the peritoneal cavity of a syngeneic mouse, for example, a mouse that has been treated (e.g., pristane-primed) to promote formation of ascites fluid containing the monoclonal antibody. Monoclonal antibodies can be isolated and purified by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104 (The Humana Press, Inc. 1992)). Monoclonal antibodies may be purified by affinity chromatography using an appropriate ligand selected based on particular properties of the antibody (e.g., heavy or light chain isotype, binding specificity, etc.). Examples of a suitable ligand, immobilized on a solid support, include Protein A, Protein G, an anticonstant region (light chain or heavy chain) antibody, an anti-idiotype antibody, and a TGF-beta binding protein, or fragment or variant thereof.

Molecular evolution of the complementarity determining regions (CDRs) in the center of the antibody binding site also has been used to isolate antibodies with increased affinity, for example, those as described by Schier et al., 1996, J. Mol. Biol. 263:551. Accordingly, such techniques are useful in preparing antibodies of the present disclosure.

Although human, partially human, or humanized antibodies will be suitable for many applications, particularly those involving administration of the antibody to a human subject, other types of antibodies will be suitable for certain applications. The non-human antibodies of the present disclosure can be, for example, derived from any antibody-producing animal, such as mouse, rat, rabbit, goat, donkey, or non-human primate (for example, monkey such as cynomolgus or rhesus monkey, or ape (e.g., chimpanzee)). Non-human antibodies of the present disclosure can be used, for example, in in vitro and cell-culture based applications, or any other application where an immune response to the antibody of the present disclosure does not occur, is insignificant, can be prevented, is not a concern, or is desired. An antibody from a particular species can be made by, for example, immunizing an animal of that species with the desired immunogen or using an artificial system for generating antibodies of that species (e.g., a bacterial or phage display-based system for generating antibodies of a particular species), or by converting an antibody from one species into an antibody from another species by replacing, e.g., the constant region of the antibody with a constant region from the other species, or by replacing one or more amino acid residues of the antibody so that it more closely resembles the sequence of an antibody from the other species. In one embodiment, the antibody is a chimeric antibody comprising amino acid sequences derived from antibodies from two or more different species.

Antibodies also may be prepared by any of a number of other conventional techniques. For example, they may be purified from cells that naturally express them (e.g., an antibody can be purified from a hybridoma that produces it), or produced in recombinant expression systems, using any technique known in the art. See, for example, Monoclonal Antibodies, Hybridomas: A New Dimension in Biological Analyses, Kenneth et al. (eds.), Plenum Press, New York (1980); and Antibodies: A Laboratory Manual, Harlow and Land (eds.), Cold Spring Harbor Laboratory Press, Cold Spring Harbor, N.Y., (1988).

Where it is desired to improve the affinity of antibodies according to the present disclosure containing one or more of the above-mentioned CDRs can be obtained by a number of affinity maturation protocols including maintaining the CDRs (Yang et al., J. Mol. Biol., 254, 392-403, 1995), chain shuffling (Marks et al., Bio/Technology, 10, 779-783, 1992), use of mutation strains of E. coli. (Low et al., J. Mol. Biol., 250, 350-368, 1996), DNA shuffling (Patten et al., Curr. Opin. Biotechnol., 8, 724-733, 1997), phage display (Thompson et al., J. Mol. Biol., 256, 7-88, 1996) and additional PCR techniques (Crameri, et al., Nature, 391, 288-291, 1998). All of these methods of affinity maturation are discussed by Vaughan et al. (Nature Biotechnology, 16, 535-539, 1998).

Single chain antibodies may be formed by linking heavy and light chain variable domain (Fv region) fragments via an amino acid bridge (short peptide linker), resulting in a single polypeptide chain. Such single-chain Fvs (scFvs) have been prepared by fusing DNA encoding a peptide linker between DNAs encoding the two variable domain polypeptides (V_{L} and V_{H}). The resulting polypeptides can fold back on themselves to form antigen-binding monomers, or they can form multimers (e.g., dimers, trimers, or tetramers), depending on the length of a flexible linker between the two variable domains (Kortt et al., 1997, Prot. Eng. 10:423; Kortt et al., 2001, Biomol. Eng. 18:95-108). By combining different VL and VH-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol Biol. 178:379-87.

Antigen binding fragments derived from an antibody can also be obtained, for example, by proteolytic hydrolysis of the antibody, for example, pepsin or papain digestion of whole antibodies according to conventional methods. By way of example, antibody fragments can be produced by enzymatic cleavage of antibodies with pepsin to provide a 5S fragment termed F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using papain produces two monovalent Fab fragments and an Fc fragment directly. These methods are described, for example, by Goldenberg, U.S. Pat. No. 4,331,647, Nisonoff et al., Arch. Biochem. Biophys. 89:230, 1960; Porter, Biochem. J. 73:119, 1959; Edelman et al., in Methods in Enzymology 1:422 (Academic Press 1967); and by Andrews, S. M. and Titus, J. A. in Current Protocols in Immunology (Coligan J. E., et al., eds), John Wiley & Sons, New York (2003), pages 2.8.1-2.8.10 and 2.10A.1-2.10A.5. Other methods for cleaving antibodies, such as separating heavy chains to form monovalent light-heavy chain fragments (Fd), further cleaving of fragments, or other enzymatic, chemical, or genetic techniques may also be used, so long as the fragments bind to the antigen that is recognized by the intact antibody.

Another exemplary form of an antibody is a peptide comprising one or more complementarity determining regions (CDRs) of an antibody. CDRs can be obtained by constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology 2:106, 1991; Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley-Liss, Inc. 1995)). The antibody fragment further may comprise at least one variable region domain of an antibody described herein. Thus, for example, the V region domain may be monomeric and be a VH or VL domain, which is capable of independently binding a desired target (e.g., human PCSK9) with an affinity at least equal to 10⁻⁷ M or less as described herein.

The variable region may be any naturally occurring variable domain or an engineered version thereof. By engineered version is meant a variable region that has been created using recombinant DNA engineering techniques. Such engineered versions include those created, for example, from a specific antibody variable region by insertions, deletions, or changes in or to the amino acid sequences of the specific antibody. One of ordinary skill in the art can use any known methods for identifying amino acid residues appropriate for engineering. Additional examples include engineered variable regions containing at least one CDR and optionally one or more framework amino acids from a first antibody and the remainder of the variable region domain from a second antibody. Engineered versions of antibody variable domains may be generated by any number of techniques with which those having ordinary skill in the art will be familiar.

The variable region may be covalently attached at a C-terminal amino acid to at least one other antibody domain or a fragment thereof. Thus, for example, a VH that is present in the variable region may be linked to an immunoglobulin CH1 domain. Similarly a VL domain may be linked to a C_{K} domain. In this way, for example, the antibody may be a Fab fragment wherein the antigen binding domain contains associated VH and VL domains covalently linked at their C-termini to a CH1 and C_{K} domain, respectively. The CH1 domain may be extended with further amino acids, for example to provide a hinge region or a portion of a hinge region domain as found in a Fab′ fragment, or to provide further domains, such as antibody CH2 and CH3 domains.

### Derivatives and Variants

The nucleotide sequences of the anti-PCSK9 antibodies of the present disclosure, encoding the corresponding amino acid sequences of the antibodies of the present disclosure, can be altered, for example, by random mutagenesis or by site-directed mutagenesis (e.g., oligonucleotide-directed site-specific mutagenesis) to create an altered polynucleotide comprising one or more particular nucleotide substitutions, deletions, or insertions as compared to the non-mutated polynucleotide. Examples of techniques for making such alterations are described in Walder et al., 1986, Gene 42:133; Bauer et al. 1985, Gene 37:73; Craik, BioTechniques, January 1985, 12-19; Smith et al., 1981, Genetic Engineering: Principles and Methods, Plenum Press; and U.S. Pat. Nos. 4,518,584 and 4,737,462. These and other methods can be used to make, for example, derivatives of the antibodies that have a desired property, for example, increased affinity, avidity, or specificity for a desired target, increased activity or stability in vivo or in vitro, or reduced in vivo side-effects as compared to the underivatized antibody.

Other derivatives of the antibodies within the scope of this disclosure include covalent or aggregative conjugates of the antibodies, with other proteins or polypeptides, such as by expression of recombinant fusion proteins comprising heterologous polypeptides fused to the N-terminus or C-terminus of a polypeptide. For example, the conjugated peptide may be a heterologous signal (or leader) polypeptide, e.g., the yeast alpha-factor leader, or a peptide such as an epitope tag. Antibody-containing fusion proteins can comprise peptides added to facilitate purification or identification of antibodies (e.g., poly-His). An antibody also can be linked to the FLAG peptide as described in Hopp et al., Bio/Technology 6:1204, 1988, and U.S. Pat. No. 5,011,912. The FLAG peptide is highly antigenic and provides an epitope reversibly bound by a specific monoclonal antibody (mAb), enabling rapid assay and facile purification of expressed recombinant protein. Reagents useful for preparing fusion proteins in which the FLAG peptide is fused to a given polypeptide are commercially available (Sigma, St. Louis, Mo.).

In another embodiment, oligomers that contain one or more antibodies may be employed in certain embodiments of the present disclosure. Oligomers may be in the form of covalently-linked or non-covalently-linked dimers, trimers, or higher oligomers. Oligomers comprising two or more antibodies are contemplated for use, with one example being a homodimer. Other oligomers include heterodimers, homotrimers, heterotrimers, homotetramers, heterotetramers, etc.

One embodiment is directed to oligomers comprising multiple antibodies joined via covalent or non-covalent interactions between peptide moieties fused to the antibodies. Such peptides may be peptide linkers (spacers), or peptides that have the property of promoting oligomerization. Leucine zippers and certain polypeptides derived from antibodies are among the peptides that can promote oligomerization of antibodies attached thereto, as described in more detail below.

In particular embodiments, the oligomers comprise from two to four antibodies. The antibodies of the oligomer may be in any form, such as any of the forms described above, e.g., variants.

In one embodiment, an oligomer is prepared using polypeptides derived from immunoglobulins. Preparation of fusion proteins comprising certain heterologous polypeptides fused to various portions of antibody-derived polypeptides (including the Fc domain) has been described, e.g., by Ashkenazi et al., 1991, PNAS USA 88:10535; Byrn et al., 1990, Nature 344:677; and Hollenbaugh et al., 1992 “Construction of immunoglobulin Fusion Proteins”, in Current Protocols in Immunology, Suppl. 4, pages 10.19.1-10.19.11.

One embodiment of the present disclosure is directed to a dimer comprising two fusion proteins created by fusing an antigen binding fragment of an anti-PCSK9 antibody to the Fc region of an antibody. The dimer can be made by, for example, inserting a gene fusion encoding the fusion protein into an appropriate expression vector, expressing the gene fusion in host cells transformed with the recombinant expression vector, and allowing the expressed fusion protein to assemble much like antibody molecules, whereupon interchain disulfide bonds form between the Fc moieties to yield the dimer.

The term “Fc polypeptide” as used herein includes native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns.

One suitable Fc polypeptide, described in PCT application WO 93/10151 (hereby incorporated by reference), is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., 1994, EMBO J. 13:3992-4001. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

In some embodiments, the variable portion of the heavy and/or light chains of a desired antibody may be substituted for the variable portion of an antibody heavy and/or light chain.

Alternatively, the oligomer is a fusion protein comprising multiple antibodies, with or without peptide linkers (spacer peptides). Among the suitable peptide linkers are those described in U.S. Pat. Nos. 4,751,180 and 4,935,233.

Another method for preparing oligomeric antibodies involves use of a leucine zipper. Leucine zipper domains are peptides that promote oligomerization of the proteins in which they are found. Leucine zippers were originally identified in several DNA-binding proteins (Landschulz et al., 1988, Science 240:1759), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., 1994, FEBS Letters 344:191, hereby incorporated by reference. The use of a modified leucine zipper that allows for stable trimerization of a heterologous protein fused thereto is described in Fanslow et al., 1994, Semin. Immunol. 6:267-78. In one approach, recombinant fusion proteins comprising a desired antibody fragment or derivative fused to a leucine zipper peptide are expressed in suitable host cells, and the soluble oligomeric antibody fragments or derivatives that form are recovered from the culture supernatant.

In another embodiment, the antibodies can be conjugated to a suitable vehicle to enhance the half-life thereof. Suitable vehicles include, but are not limited to Fc, albumin, transferrin, and the like. These and other suitable vehicles are known in the art. Such conjugated vehicles may be in monomeric, dimeric, tetrameric, or other form. In one embodiment, one or more water-soluble polymer is bonded at one or more specific position, for example at the amino terminus, of a binding agent. In an example, an antibody derivative comprises one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative comprises one or more of monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers. In certain embodiments, one or more water-soluble polymer is randomly attached to one or more side chains. In certain embodiments, PEG can act to improve the therapeutic capacity for a binding agent, such as an antibody. Certain such methods are discussed, for example, in U.S. Pat. No. 6,133,426, which is hereby incorporated by reference for any purpose. In certain embodiments, antibodies of the present disclosure may be chemically bonded with polymers, lipids, or other moieties.

### Antibody Production

The antibodies of the present disclosure can be produced by any suitable option for the synthesis of proteins (e.g., antibodies), in particular, by chemical synthesis or preferably, by recombinant expression techniques.

Recombinant expression of the antibodies requires construction of an expression vector containing a polynucleotide that encodes the antibodies. Once a polynucleotide encoding the antibody molecule has been obtained, the vector for the production of the antibodies may be produced by recombinant DNA technology. An expression vector is constructed containing the antibody coding sequences and appropriate transcriptional and translational control signals. These methods include, for example, in vitro recombinant DNA techniques, synthetic techniques, and in vivo genetic recombination.

The expression vector is transferred to a host cell by conventional techniques and the transfected cells are then cultured by conventional techniques to produce antibodies of the present disclosure. In one embodiment, vectors encoding both the heavy and light chains of an antibody may be co-expressed in the host cell for expression of the entire immunoglobulin molecule, as detailed below.

A variety of host-expression vector systems may be utilized to express the antibodies of the present disclosure. Such host-expression systems represent vehicles by which the coding sequences of interest may be produced and subsequently purified, but also represent cells which may, when transformed or transfected with the appropriate nucleotide coding sequences, express an antibody molecule of the present disclosure in situ. Bacterial cells such as E. coli, and eukaryotic cells are commonly used for the expression of a recombinant antibody molecule, especially for the expression of whole recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, COS, 293, 3T3, or myeloma cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk, hgprt or aprt-cells, respectively. Also, antimetabolite resistance can be used as the basis of selection for the following genes: dhfr, which confers resistance to methotrexate (Wigler et al., Proc. Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Wu and Wu, Biotherapy 3:87-95 (1991)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, “The use of vectors based on gene amplification for the expression of cloned genes in mammalian cells” (DNA Cloning, Vol. 3. Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors, for example, the first vector encoding an antibody heavy chain derived polypeptide and the second vector encoding an antibody light chain derived polypeptide. The two vectors may contain identical selectable markers which enable equal expression of heavy and light chain polypeptides. Alternatively, a single vector may be used which encodes, and is capable of expressing, for example, both antibody heavy and light chain polypeptides. In such situations, the light chain should be placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an antibody molecule of the present disclosure has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and size-exclusion chromatography), centrifugation, differential solubility, or by any other standard technique for the purification of proteins. In addition, the antibodies of the present disclosure or fragments thereof can be fused to heterologous polypeptide sequences described herein or otherwise known in the art, to facilitate purification.

In some embodiments, the present disclosure encompasses antibodies recombinantly fused or chemically conjugated (including both covalently and non-covalently conjugations) to a polypeptide. Fused or conjugated antibodies of the present disclosure may be used for ease in purification. See e.g., Harbor et al., supra, and PCT publication WO 93/21232; EP 439,095; Naramura et al., Immunol. Lett. 39:91-99 (1994); U.S. Pat. No. 5,474,981; Gillies et al., Proc. Natl. Acad. Sci. 89:1428-1432 (1992); Fell et al., J. Immunol. 146:2446-2452 (1991).

Moreover, the antibodies or fragments thereof of the present disclosure can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

### Antibody Effector Function

In some embodiments, the present disclosure provides antibodies with altered effector function (e.g., decreasing or increasing effector function). Nonlimiting examples of methods for increasing effector function can be found in U.S. Pat. Nos. 5,624,821, 6,602,684, 7,029,872, U.S. Patent Application Publication Nos. 2006/0067930A1, 2005/0272128A1, 2005/0079605A1, 2005/0123546A1, 2004/0072290A1, 2006/0257399A1, 2004/0261148A1, 2007/0092521, 2006/0040325A1, and 2006/0039904A1, and International Patent Application Publication Nos. WO 04/029207, WO03011878, WO05044859, WO 06071856, and WO 06071280.

Methods of engineering Fc regions of antibodies so as to alter effector functions are known in the art (e.g., U.S. Patent Publication No. 20040185045 and PCT Publication No. WO 2004/016750, both to Koenig et al., which describe altering the Fc region to enhance the binding affinity for Fc gamma RIIB as compared with the binding affinity for FC gamma RIA; see, also, PCT Publication Nos. WO 99/58572 to Armour et al., WO 99/51642 to Idusogie et al., and U.S. Pat. No. 6,395,272 to Deo et al.). Methods of modifying the Fc region to decrease binding affinity to Fc gamma RIIB are also known in the art (e.g., U.S. Patent Publication No. 20010036459 and PCT Publication No. WO 01/79299, both to Ravetch et al.). Modified antibodies having variant Fc regions with enhanced binding affinity for Fc gamma RIIIA and/or Fc gamma RIA as compared with a wildtype Fc region have also been described (e.g., PCT Publication Nos. WO 2004/063351, to Stavenhagen et al., the disclosure of which is incorporated herein in its entirety).

Antibody effector function may also be modified through the generation of antibodies with altered glycosylation patterns. Such altered glycosylation patterns have been demonstrated to increase or decrease the ADCC ability of antibodies, as desired. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present disclosure to thereby produce an antibody with altered glycosylation.

### Half-Life Alteration

In some embodiments, the present disclosure provides for antibodies which have an extended half-life in vivo. In particular, the present disclosure provides antibodies which have a half-life in a mammal (for example, but not limited to, a human), of greater than 3 days, greater than 7 days, greater than 10 days, greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of antibodies (for example, monoclonal antibodies) or antibody fragments (for example, Fab fragments) in vivo, for example, inert polymer molecules such as high molecular weight polyethyleneglycol (PEG) can be attached to the antibodies (including antibody fragments thereof) with or without a multifunctional linker either through site-specific conjugation of the PEG to the N- or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods known to those of skill in the art, for example, by immunoassays described herein.

In certain embodiments, antibodies having an increased half-life in vivo can also be generated by introducing one or more amino acid modifications (i.e., substitutions, insertions or deletions) into an IgG constant domain, or FcRn binding fragment thereof (e.g., Fc or hinge Fc domain fragment). See, e.g., International Publication No. WO 98/23289; International Publication No. WO 97/34631; and U.S. Pat. No. 6,277,375, each of which is incorporated herein by reference in its entirety.

In some embodiments, covalent modifications of the antibodies of the present disclosure are included within the scope of the disclosed subject matter. They may be made by chemical synthesis or by enzymatic or chemical cleavage of the antibodies, if applicable. Other types of covalent modifications of the antibodies are introduced into the molecule by reacting targeted amino acid residues of the antibody with an organic derivatizing agent that is capable of reacting with selected side chains or the N- or C-terminal residues.

### Inhibitory Nucleic Acids

PCSK9 inhibitors can also include RNAi therapies, such as siRNA, for example inclisiran (ALN-PCSsc). In some embodiments, the PCSK9 inhibitor includes the specific double stranded sequence of ALN-PCSsc (from U.S. Pat. Nos. 7,605,251, 8,809,292, 9,260,718 and 8,273,869). In some embodiments, the PCSK9 inhibitor includes polynucleotide compositions that target PCSK9 and are useful for methods for treatment, therapy, and prophylaxis in disease related to PCSK9 expression, where reduction or inhibition of the expression or function of a selected target polynucleotide sequence is desired. Examples of inhibitory nucleic acids that can be used to target PCSK9 sequences and reduce PCSK9 expression include, but are not limited to, antisense oligonucleotides, and RNA interference (RNAi) agents, including short or small interfering RNA (siRNA), short hairpin RNA (shRNA), and microRNA (miRNA). See, for example, U.S. Pat. Nos. 6,506,559; 8,394,628; 7,056,704; 7,078,196; 6,107,094; 5,898,031; 6,573,099; and European Patent No. 1,144,623. See also, for example, U.S. patent application publication nos. 2015/0259689; 2015/0197746; 2011/0092565; U.S. Pat. Nos. 8,877,917; 8,507,455; and 7,579,451. See also, for example, International Publication No. WO 2014/089313 and WO 2018/075658. In some embodiments, the PCSK9 inhibitor is inclisiran, as described in International Publication No. WO 2014/089313.

In certain embodiments, an inhibitory nucleic acid that inhibits the function or expression of a target polynucleotide sequence (e.g. PCSK9 mRNA sequence) in a mammalian cell, according to the present disclosure, comprises an agent that provides to a mammalian cell an at least partially double-stranded RNA molecule (e.g., an interfering RNA molecule). A double-stranded RNA molecule may include chemical modifications to ribonucleotides, including modifications to the ribose sugar, base, or backbone components of the ribonucleotides, such as those described herein or known in the art. Any such modifications, as used in a double-stranded RNA molecule (e.g. siRNA, shRNA, or the like), are encompassed by the term “double-stranded RNA” for the purposes of this disclosure. Thus, in general, the term “RNA” may also include RNA-DNA hybrids and polynucleotides comprising one or more modified nucleotides (e.g. nucleotides with modifications at the 2′ position of the ribose ring), except where specified otherwise, e.g., where a 2′-OH group of ribose is required for a particular linkage.

In some embodiments at least 10% of a partially double-stranded RNA molecule is double-stranded. Alternatively, the double stranded portion of these RNA molecules can be at least 30% of the length of the molecule. In another embodiment, the double stranded portion of these molecules can be at least 50% of the length of the molecule. In still another embodiment, the double stranded portion of these molecules can be at least 70% of the length of the molecule. In another embodiment, the double stranded portion of these molecules can be at least 90% of the length of the molecule. In another embodiment, the molecule can be double stranded over its entire length. Alternatively, the double-stranded portion of these molecules can occur at either or both termini, or in some middle portion of the molecule, if the molecule is linear. Similarly, the double-stranded portion can be in any location if the molecule is circular. In certain embodiments of the present disclosure, the double-stranded portion of the RNA molecule becomes double-stranded only when the molecule is in the mammalian cell. In still other embodiment of the present disclosure, the partially double-stranded molecule is an RNA/DNA hybrid, for example, a single strand containing RNA and DNA, prepared in vitro; or a duplex of two such single strands or portions thereof. In yet another embodiment, the RNA molecule, made in vivo or in vitro, is a duplex comprised of an RNA single strand and a DNA single strand. In some embodiments, the partially double-stranded RNA molecule comprises a polynucleotide sequence that is substantially homologous to the target polynucleotide sequence in order to effectively reduce or inhibit the function or expression thereof. The necessary homology may be suitably defined by use of a computer algorithm.

As known in the art and discussed herein, “homology” or “identity” means the degree of sequence relatedness between two polypeptide or two polynucleotide sequences as determined by the identity of the match between two lengths of such sequences. Both identity and homology can be readily calculated by methods in the prior art [See also, e.g., COMPUTATIONAL MOLECULAR BIOLOGY, Lesk, A. M., ed., Oxford University Press, New York, (1988); BIOCOMPUTING: INFORMATICS AND GENOME PROJECTS, Smith, D. W., ed., Academic Press, New York, (1993); COMPUTER ANALYSIS OF SEQUENCE DATA, PART I, Griffin, A. M., and Griffin, H. G., eds., Humana Press, New Jersey, (1994); SEQUENCE ANALYSIS IN MOLECULAR BIOLOGY, von Heinje, G., Academic Press, (1987); and SEQUENCE ANALYSIS PRIMER, Gribskov, M. and Devereux, J., eds., M Stockton Press, New York, (1991)]. While there exist a number of methods to measure identity and homology between two polynucleotide sequences, the terms “identity”, “similarity” and homology are well known to skilled artisans [H. Carillo and D. Lipton, SIAM J. Applied Math., 48:1073 (1988)]. Methods commonly employed to determine identity or homology between two sequences include, but are not limited to, those disclosed in Guide to Huge Computers, Martin J. Bishop, ed., Academic Press, San Diego, 1994, and H. Carillo and D. Lipton, SIAM J. Applied Math., 48:1073 (1988). Preferred methods to determine identity or homology are designed to give the largest match between the two sequences tested. Methods to determine identity and similarity are codified in computer programs. Preferred computer program to determine identity and homology between two sequences include, but are not limited to, the algorithm BESTFIT from the GCG program package [J. Devereux et al., Nucl. Acids Res., 12(1):387 (1984)], the related MACVECTOR program (Oxford), and the FASTA (Pearson) programs. For instance, searches for sequence similarities in databases between significant naturally occurring mammalian polynucleotide sequences and target polynucleotide sequences enable the design of suitable RNA molecules desired for use in the present disclosure. The algorithm and/or the degree of homology necessary for any particular RNA molecule may be selected by one of skill in the art, depending on the identity of the target, and/or the closeness of homology of the target sequence to any naturally occurring mammalian sequence, which is desired to be left functioning normally after use of the methods of the present disclosure.

In some embodiments, an inhibitory nucleic acid for reducing the expression or function of PCSK9 sequences is an RNAi agent comprising a double-stranded RNA molecule which comprises two antiparallel strands of contiguous nucleotides that are sufficiently complementary to each other to hybridize to form a duplex region. “Hybridize” or “hybridization” refers to the pairing of complementary polynucleotides, typically via hydrogen bonding (e.g. Watson-Crick, Hoogsteen or reversed Hoogsteen hydrogen bonding) between complementary bases in the two polynucleotides. The strand comprising a region having a sequence that is substantially complementary to a target sequence (e.g. target mRNA) is referred to as the “antisense strand.” The “sense strand” refers to the strand that includes a region that is substantially complementary to a region of the antisense strand. In some embodiments, the sense strand may comprise a region that has a sequence that is substantially identical to the target sequence.

As used herein, a first sequence is “complementary” to a second sequence if a polynucleotide comprising the first sequence can hybridize to a polynucleotide comprising the second sequence to form a duplex region under certain conditions, such as physiological conditions. Other such conditions can include moderate or stringent hybridization conditions, which are known to those of skill in the art. A first sequence is considered to be fully complementary (100% complementary) to a second sequence if a polynucleotide comprising the first sequence base pairs with a polynucleotide comprising the second sequence over the entire length of one or both nucleotide sequences without any mismatches. A sequence is “substantially complementary” to a target sequence if the sequence is at least 80%, 85%, 90%, 95%, 96%, 97%, 98%, 99% or 100% complementary to a target sequence. Percent complementarity can be calculated by dividing the number of bases in a first sequence that are complementary to bases at corresponding positions in a second or target sequence by the total length of the first sequence. A sequence may also be said to be substantially complementary to another sequence if there are no more than 5, 4, 3, or 2 mismatches over a 30 base pair duplex region when the two sequences are hybridized. Generally, if any nucleotide overhangs, as defined herein, are present, the sequence of such overhangs is not considered in determining the degree of complementarity between two sequences. By way of example, a sense strand of 21 nucleotides in length and an antisense strand of 21 nucleotides in length that hybridize to form a 19 base pair duplex region with a 2 nucleotide overhang at the 3′ end of each strand would be considered to be fully complementary as the term is used herein.

In some embodiments, a region of the antisense strand comprises a sequence that is fully complementary to a region of the target RNA sequence (e.g. PCSK9 mRNA, such as human PCSK9 mRNA). In such embodiments, the sense strand may comprise a sequence that is fully complementary to the sequence of the antisense strand. In other such embodiments, the sense strand may comprise a sequence that is substantially complementary to the sequence of the antisense strand, e.g. having 1, 2, 3, 4, or 5 mismatches in the duplex region formed by the sense and antisense strands. In certain embodiments, it is preferred that any mismatches occur within the terminal regions (e.g. within 6, 5, 4, 3, or 2 nucleotides of the 5′ and/or 3′ ends of the strands). In one embodiment, any mismatches in the duplex region formed from the sense and antisense strands occur within 6, 5, 4, 3, or 2 nucleotides of the 5′ end of the antisense strand.

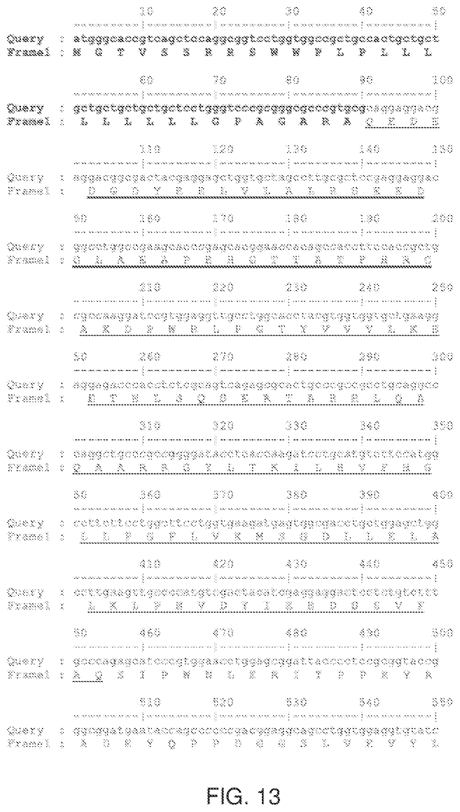

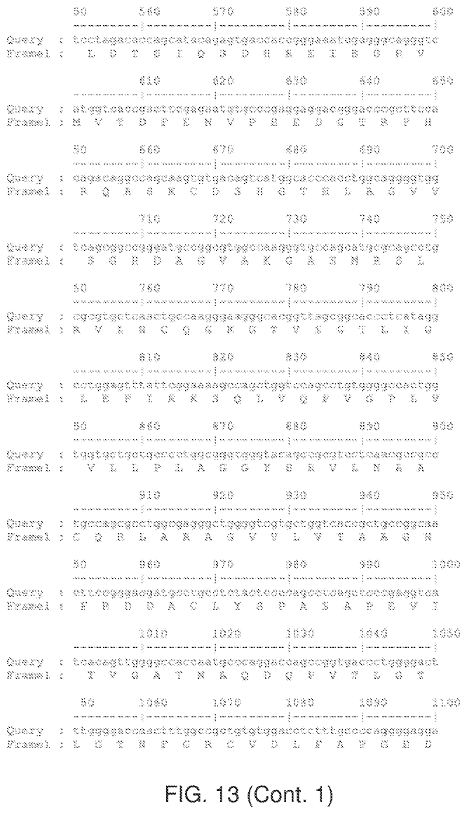

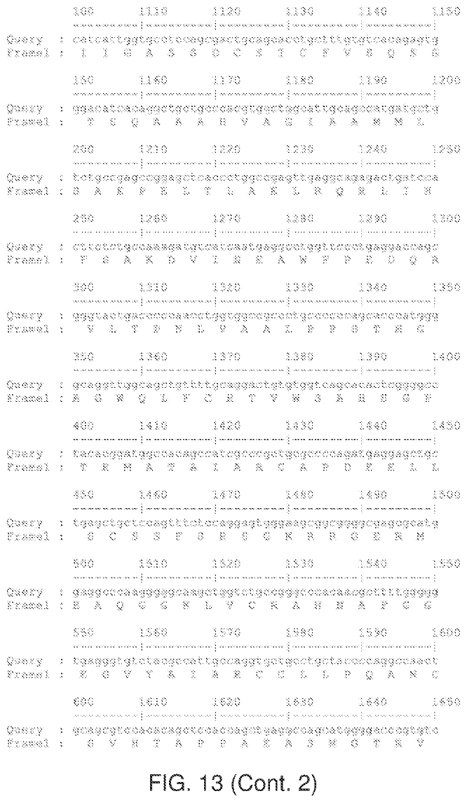

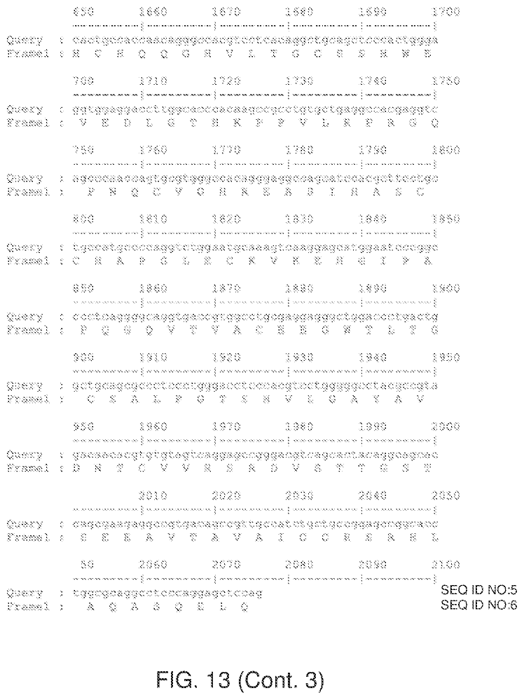

In some embodiments, an inhibitory nucleic acid that targets PCSK9 sequences and reduce PCSK9 expression includes, without limitation, a polynucleotide sequence that is fully, or substantially, complementary to at least a portion of a human PCSK9 mRNA sequence. In some embodiments, an inhibitory nucleic acid that targets PCSK9 sequences and reduce PCSK9 expression includes an antisense strand that is fully complementary to at least a portion of an RNA sequence encoded by the nucleotide sequence shown in FIGS. 11 and 13 (SEQ ID NOS: 3, 5). In some embodiments, an inhibitory nucleic acid that targets PCSK9 sequences and reduce PCSK9 expression includes an antisense strand that is substantially complementary to at least a portion of an RNA sequence encoded by the nucleotide sequence shown in FIGS. 11 and 13 (SEQ ID NOS: 3, 5), e.g. having 1, 2, 3, 4, or 5 mismatches in the duplex region formed by the sense and antisense strands. The region of full or substantial complementarity with the target PCSK9 mRNA can be any suitable length. In some embodiments, the region of full or substantial complementarity with the target PCSK9 mRNA is 18, 19, 20, 21, 22, 23, 24, 25, 26, 27, or 28 nucleotides in length.

In certain embodiments, the sense strand and antisense strand of the double-stranded RNA may be two separate molecules that hybridize to form a duplex region, but are otherwise unconnected. Such double-stranded RNA molecules formed from two separate strands are referred to as “small interfering RNAs” or “short interfering RNAs” (siRNAs).

### Inhibitory Nucleic Acid Delivery

The inhibitory nucleic acids (e.g., interfering RNA such as siRNA) can be administered by any method suitable for administration of nucleic acid agents, such as a DNA vaccine or gene therapy vectors. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from, for example, Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

The interfering RNA molecule may be conjugated to one or more carbohydrate moieties to optimize one or more properties of the interfering RNA molecule. In many cases, the carbohydrate moiety will be attached to a modified subunit of the interfering RNA molecule or at the 5′ or 3′ end of one of strands of the interfering RNA molecule. E.g., the ribose sugar of one or more ribonucleotide subunits of an interfering RNA molecule can be replaced with another moiety, e.g., a non-carbohydrate (preferably cyclic) carrier to which is attached a carbohydrate moiety. A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The carbohydrate moiety may be attached to the polynucleotide via a carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

In some embodiments the inhibitory nucleic acid, e.g., interfering RNA molecule, is conjugated to a carbohydrate moiety via a carrier, wherein the carrier can be cyclic group or acyclic group; in specific embodiments, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalin; preferably, the acyclic group is selected from serinol backbone or diethanolamine backbone.

### Targeting Inhibitory Nucleic Acids

In some embodiments, the inhibitory nucleic acid, e.g., interfering RNA molecules, are targeted to tissues of interest, e.g., the liver. In some embodiments, the inhibitory nucleic acid, e.g., interfering RNA, is delivered to the liver. Accordingly, in certain embodiments, the inhibitory nucleic acid is specifically targeted to liver cells using various methodologies known in the art and described herein. For example, in certain embodiments, antibodies or other targeting moieties disclosed herein below can be used to specifically target the inhibitory nucleic acid to the hepatocytes using various different receptors expressed on the surface of hepatocytes.

A wide variety of targeting moieties can be coupled to the oligonucleotides of the present disclosure. In some embodiments, the targeting moieties are coupled, e.g., covalently, either directly or indirectly via an intervening tether.

In some embodiments, a targeting moiety alters the distribution, targeting or lifetime of the molecule into which it is incorporated. In preferred embodiments a targeting moiety provides an enhanced affinity for a selected target, e.g., molecule, cell or cell type, compartment, receptor e.g., a cellular or organ compartment, tissue, organ or region of the body, as, e.g., compared to a species absent such a targeting moiety. Targeting moieties providing enhanced affinity for a selected target are also termed targeting moieties.

Some targeting moieties can have endosomolytic properties. The endosomolytic targeting moieties promote the lysis of the endosome and/or transport of the composition of the present disclosure, or its components, from the endosome to the cytoplasm of the cell. The endosomolytic targeting moiety may be a polyanionic peptide or peptidomimetic which shows pH-dependent membrane activity and fusogenicity. In one embodiment, the endosomolytic targeting moiety assumes its active conformation at endosomal pH. The “active” conformation is that conformation in which the endosomolytic targeting moiety promotes lysis of the endosome and/or transport of the composition of the present disclosure, or its components, from the endosome to the cytoplasm of the cell. Exemplary endosomolytic targeting moieties include the GALA peptide (Subbarao et al., Biochemistry, 1987, 26: 2964-2972), the EALA peptide (Vogel et al., J. Am. Chem. Soc., 1996, 118: 1581-1586), and their derivatives (Turk et al., Biochem. Biophys. Acta, 2002, 1559: 56-68). In one embodiment, the endosomolytic component may contain a chemical group (e.g., an amino acid) which will undergo a change in charge or protonation in response to a change in pH. The endosomolytic component may be linear or branched.

In certain embodiments, targeting moieties can improve transport, hybridization, and specificity properties and may also improve nuclease resistance of the resultant natural or modified oligoribonucleotide, or a polymeric molecule comprising any combination of monomers described herein and/or natural or modified ribonucleotides.

In some embodiments, targeting moieties in general can include therapeutic modifiers, e.g., for enhancing uptake; diagnostic compounds or reporter groups e.g., for monitoring distribution; cross-linking agents; and nuclease-resistance conferring moieties. General examples include lipids, steroids, vitamins, sugars, proteins, peptides, polyamines, and peptide mimics.

Targeting moieties can include a naturally occurring substance, such as a protein (e.g., human serum albumin (I), low-density lipoprotein (LDL), high-density lipoprotein (HDL), or globulin); a carbohydrate (e.g., a dextran, pullulan, chitin, chitosan, inulin, cyclodextrin or hyaluronic acid); or a lipid. The targeting moiety may also be a recombinant or synthetic molecule, such as a synthetic polymer, e.g., a synthetic polyamino acid, an oligonucleotide (e.g. an aptamer). Examples of polyamino acids include polyamino acid is a polylysine (PLL), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Targeting moieties can also include other targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptide mimetic or an aptamer.

Other examples of targeting moieties include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases or a chelator (e.g. EDTA), lipophilic molecules, e.g., cholesterol, cholic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Targeting moieties can be proteins, e.g., glycoproteins, or peptides, e.g., molecules having a specific affinity for a co-moiety, or antibodies; e.g., an antibody that binds to a specified cell type such as a liver hepatocyte. Targeting moieties may also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, multivalent fucose, or aptamers. The targeting moiety can be, for example, a lipopolysaccharide.

The targeting moiety can be a substance, e.g., a drug, which can increase the uptake of the inhibitory nucleic acid, e.g., interfering RNA molecule, into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoseverin.

The targeting moiety can increase the uptake of the inhibitory nucleic acid, e.g., interfering RNA molecule into the cell by activating an inflammatory response, for example. Exemplary targeting moieties that would have such an effect include tumor necrosis factor alpha (TNFalpha), interleukin-1 beta, or gamma interferon.

### Synthesis of Interfering RNA

The interfering RNA molecules that can be employed in the methods of the present disclosure can readily be made using techniques known in the art, for example, using conventional RNA solid phase synthesis. See, for example, U.S. Pat. No. 8,877,917. The polynucleotides of the double-stranded RNA molecules can be assembled on a suitable nucleic acid synthesizer utilizing standard nucleotide or nucleoside precursors (e.g. phosphoramidites). Automated nucleic acid synthesizers are sold commercially by several vendors, including DNA/RNA synthesizers from Applied Biosystems (Foster City, Calif.), MerMade synthesizers from BioAutomation (Irving, Tex.), and OligoPilot synthesizers from GE Healthcare Life Sciences (Pittsburgh, Pa.).

The 2′ silyl protecting group can be used in conjunction with acid labile dimethoxytrityl (DMT) at the 5′ position of ribonucleosides to synthesize oligonucleotides via phosphoramidite chemistry. Final deprotection conditions are known not to significantly degrade RNA products. All syntheses can be conducted in any automated or manual synthesizer on large, medium, or small scale. The syntheses may also be carried out in multiple well plates or glass slides.

The 2′-O-silyl group can be removed via exposure to fluoride ions, which can include any source of fluoride ion, e.g., those salts containing fluoride ion paired with inorganic counterions e.g., cesium fluoride and potassium fluoride or those salts containing fluoride ion paired with an organic counterion, e.g., a tetraalkylammonium fluoride. A crown ether catalyst can be utilized in combination with the inorganic fluoride in the deprotection reaction. Preferred fluoride ion source are tetrabutylammonium fluoride or aminehydrofluorides (e.g., combining aqueous HF with triethylamine in a dipolar aprotic solvent, e.g., dimethylformamide).

The choice of protecting groups for use on the phosphite triesters and phosphotriesters can alter the stability of the triesters towards fluoride. Methyl protection of the phosphotriester or phosphitetriester can stabilize the linkage against fluoride ions and improve process yields.

Since ribonucleosides have a reactive 2′ hydroxyl substituent, it can be desirable to protect the reactive 2′ position in RNA with a protecting group that is orthogonal to a 5′-O-dimethoxytrityl protecting group, e.g., one stable to treatment with acid. Silyl protecting groups meet this criterion and can be readily removed in a final fluoride deprotection step that can result in minimal RNA degradation.

Tetrazole catalysts can be used in the standard phosphoramidite coupling reaction. Preferred catalysts include e.g., tetrazole, S-ethyl-tetrazole, p-nitrophenyltetrazole.

See also, for example, Trufert et al., Tetrahedron, 52:3005, 1996; and Manoharan, “Oligonucleotide Conjugates in Antisense Technology,” in Antisense Drug Technology, ed. S. T. Crooke, Marcel Dekker, Inc., 2001. The protected monomer compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Other synthetic chemistry transformations, protecting groups (e.g., for hydroxyl, amino, etc. present on the bases) and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

### Kits

Kits for use by medical practitioners and others are provided including one or more PCSK-9 inhibitors (e.g., antibody, inhibitory nucleic acid or small molecule inhibitor), and a label or other instructions for use in treating any of the conditions discussed herein and/or additional components. In one embodiment, the kit includes a sterile preparation of one or more human antibodies, or one or more interfering RNA which may be in the form of a composition as disclosed herein, and may be in one or more vials. Other PCSK9 inhibitors can also be employed.

Provided herein is a kit for treating a pediatric subject in need of treating or preventing heterozygous familial hypercholesterolemia (HeFH) or symptoms thereof. The kit can include a dosage form comprising a PCSK9 inhibitor in an amount sufficient to administer the PCSK9 inhibitor to a pediatric HeFH subject, e.g., a pediatric HeFH subject having a baseline LDL-C at or above an upper quartile of baseline LDL-C values among a pediatric HeFH patient cohort, an enhanced dosage regimen, as described herein, comprising administering to the patient the PCSK9 inhibitor at a dosing frequency that is at least 2 fold greater than an average dosing frequency of the PCSK9 inhibitor for pediatric HeFH patients having a baseline LDL-C value that is less than the upper quartile, e.g., less than the median of baseline LDL-C values among the cohort.

Also provided is a kit for treating a pediatric subject in need of treating or preventing heterozygous familial hypercholesterolemia (HeFH) or symptoms thereof. The kit can include a dosage form comprising a PCSK9 inhibitor in an amount sufficient to administer the PCSK9 inhibitor to a pediatric HeFH subject, e.g., a pediatric HeFH subject having a baseline LDL-C at or above an upper quartile of baseline LDL-C values among a pediatric HeFH patient cohort, an enhanced dosage regimen, as described herein, comprising administering to the patient the PCSK9 inhibitor at a dosage that is about 20% to about 500% greater than a standard-of-care dosage of the PCSK9 inhibitor to treat or prevent the cholesterol-related disorder in an adult HeFH patient.

### EXAMPLES

### Example 1

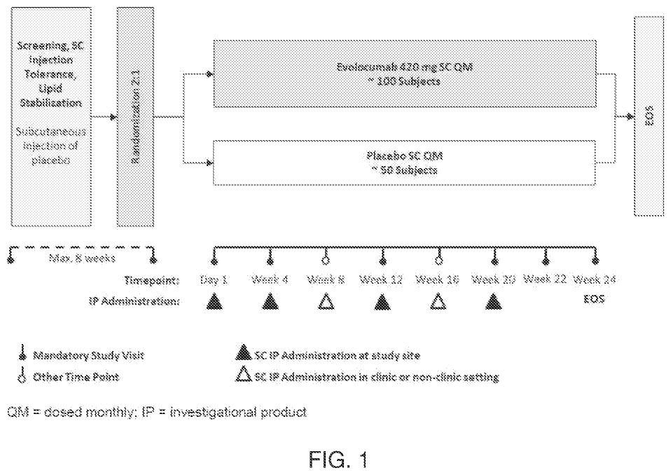

This non-limiting example shows a double-blind, randomized, multicenter, placebo-controlled, parallel group study to characterize the efficacy, safety, and tolerability of 24 weeks of evolocumab for low density lipoprotein-cholesterol (LDL-C) reduction, as add-on to diet and lipid-lowering therapy, in pediatric subjects 10 to 17 years of age with heterozygous familial hypercholesterolemia (HeFH). The study design is shown in FIG. 1. Enrolled participants were randomized to receive evolocumab (420 mg monthly) or matching placebo injections subcutaneously (FIG. 1). Subject inclusion and exclusion criteria used are listed below.

Inclusion Criteria:
Male or female≥10 to ≤17 years of age (before 18th birthday)
Diagnosis of heterozygous familial hypercholesterolemia
On an approved statin with stable optimized dose for ≥4 weeks
Other lipid-lowering therapy stable for ≥4 weeks (fibrates must be stable for 6 weeks)
Fasting LDL-C≥130 mg/dL (3.4 mmol/L)
Fasting triglycerides≤400 mg/dL (4.5 mmol/L)

Exclusion Criteria:
Type 1 diabetes, or type 2 diabetes that is or poorly controlled
Uncontrolled hyperthyroidism or hypothyroidism
Cholesterylester transfer protein (CETP) inhibitor in the last 12 months, or mipomersen or lomitapide in the last 5 months
Previously received evolocumab or any other investigational therapy to inhibit PCSK9.
Lipid apheresis within the last 12 weeks prior to screening.
Homozygous Familial Hypercholesterolemia

Primary Objective and Endpoint:

To evaluate the effect of 24 weeks subcutaneous (SC) evolocumab compared with placebo, when added to standard of care, on percent change from baseline in low-density lipoprotein cholesterol (LDL-C) in pediatric subjects 10 to 17 years of age with HeFH.

The primary endpoint was the percent change from baseline to week 24 in LDL-C.

Secondary Efficacy Objectives and Endpoints:

To assess the effects of SC evolocumab compared with placebo, when added to standard of care, on mean percent change from baseline to weeks 22 and 24 and change from baseline to week 24 in LDL-C, and on percent change from baseline to week 24 in non-high-density lipoprotein cholesterol (non-HDL-C), apolipoprotein B (ApoB), total cholesterol/HDL-C ratio, and ApoB/Apolipoprotein A-1 (ApoA1) ratio, in pediatric subjects 10 to 17 years of age with HeFH.

Secondary endpoints were the mean percent change in LDL-C from baseline at weeks 22 and 24 (tier 1); the change in LDL-C from baseline at week 24 (tier 2); and the percent change from baseline to week 24 in non-HDL-C, ApoB, total cholesterol/HDL-C ratio and ApoB/ApoA1 ratio (tier 3).

Summary of Results:

A total of 158 subjects were randomized of which 157 received at least one dose of investigational product (IP) and were included in the full analysis set with 104 receiving evolocumab (EvoMab) 420 mg SC QM and 53 receiving placebo SC QM. Median (Q1, Q3) double-blind IP exposure was 5.6 (5.5, 5.6) months in each treatment arm. 96.8% and 99.4% of the subjects completed IP and the study, respectively (see Table 1.1). The baseline characteristics of subjects in the full analysis set is shown in Table 1.2.

TABLE 1.1
Summary of Subject and Study Disposition for all Subjects Randomized

| | Placebo QM | | EvoMab 420 mg QM | | Total | |
|---|---|---|---|---|---|---|
| | (N = 53) | | (N = 105) | | (N = 158) | |
| Disposition | n(%) | | n(%) | | n(%) | |
| Investigational product accounting | | | | | | |
| Subjects who never received IP | 0 | (0.0) | 1 | (1.0) | 1 | (0.6) |
| Subjects who received IP | 53 | (100.0) | 104 | (99.0) | 157 | (99.4) |
| Subjects who completed IP | 53 | (100.0) | 100 | (95.2) | 153 | (96.8) |
| Subjects who discontinued IP | 0 | (0.0) | 4 | (3.8) | 4 | (2.5) |
| Adverse event | 0 | (0.0) | 1 | (1.0) | 1 | (0.6) |
| Subject request | 0 | (0.0) | 2 | (1.9) | 2 | (1.3) |
| Other | 0 | (0.0) | 1 | (1.0) | 1 | (0.6) |
| Study completion accounting | | | | | | |
| Subjects who completed study | 53 | (100.0) | 104 | (99.0) | 157 | (99.4) |
| Subjects who discontinued study | 0 | (0.0) | 1 | (1.0) | 1 | (0.6) |
| Withdrawal of consent from study | 0 | (0.0) | 1 | (1.0) | 1 | (0.6) |

TABLE 1.2
Baseline Characteristics for the Full Analysis Set

| | | |
|---|---|---|
| Placebo QM | EvoMab 420 mg QM | Total |
| (N = 53) | (N = 104) | (N = 157) |

Demographics

| | | | | | | |
|---|---|---|---|---|---|---|
| Age in years, mean(SD) | 13.7 | (2.5) | 13.7 | (2.3) | 13.7 | (2.4) |
| Age group, <14 years, n(%) | 25 | (47.2) | 48 | (46.2) | 73 | (46.5) |
| Sex, male, n(%) | 26 | (49.1) | 43 | (41.3) | 69 | (43.9) |
| Race, n(%) | | | | | | |
| White | 44 | (83.0) | 89 | (85.6) | 133 | (84.7) |
| Biack or African American | 0 | (0.0) | 2 | (1.9) | 2 | (1.3) |
| Asian | 0 | (0.0) | 2 | (1.9) | 2 | (1.3) |
| Other | 9 | (17.0) | 11 | (10.6) | 20 | (12.7) |
| Ethnicity, n(%) | | | | | | |
| Hispanic/Latino | 7 | (13.2) | 6 | (5.8) | 13 | (8.3) |
| Region, n(%) | | | | | | |
| Europe | 35 | (66.0) | 68 | (65.4) | 103 | (65.6) |
| Latin America | 8 | (15.1) | 18 | (17.3) | 26 | (16.6) |
| North America | 10 | (18.9) | 12 | (11.5) | 22 | (14.0) |
| Asia Pacific | 0 | (0.0) | 6 | (5.8) | 6 | (3.8) |

Cardiovascular risk factors, n(%)

| Hypertension | | 3 | (5.7) | 2 | (1.9) | 5 | (3.2) |
|---|---|---|---|---|---|---|---|
| Low HDL-C | | 18 | (34.0) | 40 | (38.5) | 58 | (36.9) |
| Current cigarette smoking | | 2 | (3.8) | 1 | (1.0) | 3 | (1.9) |
| Type II diabetes mellitus | | 0 | (0.0) | 1 | (1.0) | 1 | (0.6) |
| Family history of premature CHD | | 21 | (39.6) | 31 | (29.8) | 52 | (33.1) |
| BMI (kg/m²), mean (SD) | | 21.3 | (4.2) | 22.6 | (5.5) | 22.1 | (5.1) |
| Coronary artery disease | | 0 | (0.0) | 0 | (0.0) | 0 | (0.0) |
| Cerebrovascular or peripheral arterial | | 1 | (1.9) | 0 | (0.0) | 1 | (0.6) |
| Lipid-lowering therapy | | | | | | | |
| Statins, n(%) | | 52 | (98.1) | 104 | (100.0) | 156 | (99.4) |
| High intensity, n(%) | | 7 | (13.2) | 19 | (18.3) | 26 | (16.6) |
| Moderate intensity, n(%) | | 35 | (66.0) | 63 | (60.6) | 98 | (62.4) |
| Low intensity, n(%) | | 10 | (18.9) | 21 | (20.2) | 31 | (19.7) |
| Unknown intensity, n(%) | | 0 | (0.0) | 1 | (1.0) | 1 | (0.6) |
| Ezetimibe, n(%) | | 8 | (15.1) | 13 | (12.5) | 21 | (13.4) |
| Baseline | Mean (SD) | 183.0 | (47.2) | 185.0 | (45.0) | 184.3 | (45.6) |
| LDL-C (mg/dL) | Median (Q1, Q3) | 173.0 | (148.0, 208.5) | 172.8 | (155.0, 207.5) | 173.0 | (154.0, 208.0) |
| Baseline non- | Mean (SD) | 200.2 | (48.2) | 203.8 | (47.3) | 202.6 | (47.5) |
| HDL-C (mg/dL) | Median (Q1, Q3) | 188.5 | (164.0, 229.5) | 193.3 | (172.3, 224.8) | 192.0 | (169.0, 225.0) |

The result was statistically significant for the primary endpoint of percent change in LDL-C from baseline at week 24 (p<0.0001). EvoMab reduced LDL-C by an additional 38.300% (standard error (SE)=3.66) compared to placebo (Table 1.3).

EvoMab statistically significantly improved all of the tier 1, 2 and 3 secondary endpoints compared to placebo (Table 1.3). For the tier 1 secondary endpoint, the mean percent change in LDL-C from baseline at weeks 22 and 24, EvoMab reduced LDL-C by an additional 42.09% (SE=3.17). For the tier 2 secondary endpoint, the change in LDL-C from baseline at week 24, EvoMab reduced LDL-C by an additional 68.6 mg/dL (SE=7.3).

TABLE 1.3
Treatment Difference in Primary and Secondary Endpoints

| | | EvoMab - Placebo | |
|---|---|---|---|
| | | Least squares | Adjusted |
| | | estimate (95% CI) | pvalue |
| Primary endpoint | Percent change from baseline to week | −38.30 (−45.54, −31.06) | <0.0001 |
| | 24 in LDL-C | | |
| Tier 1 Secondary | Mean percent change from baseline to | −42.09 (−48.34, −35.83) | <0.0001 |
| endpoint | weeks 22 and 24 in LDL-C | | |
| Tier 2 Secondary | Change from baseline to week 24 in | −68.6 (−83.1, −54.0) | <0.0001 |
| endpoint | LDL-C (mg/dL) | | |
| Tier 3 Secondary | Percent change from baseline to week 24 in | | |
| endpoints | non-HDL-C | −35.04 (−41.79, −28.30) | <0.0001 |
| | ApoB | −32.47 (−38.82, −26.13) | <0.0001 |
| | total cholesterol/HDL-C ratio | −30.30 (−36.40, −24.21) | <0.0001 |
| | ApoB/ApoA1 ratio | −36.38 (−42.97, −29.80) | <0.0001 |

No new safety concerns were identified from the results of the study, and the subject incidence of treatment-emergent adverse events were comparable across both treatment groups (Table 1.4).

TABLE 1.4
Summary of Safety Results

| | | EvoMab 420 |
|---|---|---|
| | Placebo QM | mg QM |
| | (N = 53) | (N = 104) |
| Treatment Emergent Adverse Events (TEAE) | 34 (64.2) | 64 (61.5) |
| Grade ≥2 | 22 (41.5) | 46 (44.2) |
| Grade ≥3^{a} | 0 (0.0) | 4 (3.8) |
| Grade ≥4 | 0 (0.0) | 0 (0.0) |
| SAEs^{b} | 0 (0.0) | 1 (1.0) |
| TEAEs leading to discontinuation of IP | 0 (0.0) | 1 (1.0) |
| Fatal AEs (or Deaths) | 0 (0.0) | 0 (0.0) |
| Most common TEAEs (≥2% in EvoMab) | | |
| Nasopharyngitis | 6 (11.3) | 12 (11.5) |
| Headache^{c} | 1 (1.9) | 11 (10.6) |
| Oropharyngeal pain^{c} | 0 (0.0) | 7 (6.7) |
| Influenza^{c} | 2 (3.8) | 6 (5.8) |
| Upper respiratory tract infection^{c} | 1 (1.9) | 6 (5.8) |
| Gastroenteritis | 4 (7.5) | 5 (4.8) |
| Pyrexia | 3 (5.7) | 3 (2.9) |
| Constipation | 0 (0.0) | 3 (2.9) |
| Influenza like illness^{c} | 0 (0.0) | 3 (2.9) |
| EOI: Potential hypersensitivity events | 0 (0.0) | 4 (3.8) |
| (narrow terms)^{d} | | |
| EOI: Potential hypersensitivity events | 0 (0.0) | 7 (6.7) |
| (broad terms)^{d} | | |
| EOI: Potential injection site reaction events | 3 (5.7) | 5 (4.8) |
| (narrow terms) | | |
| EOI: Potential injection site reaction events | 3 (5.7) | 5 (4.8) |
| (broad terms) | | |
| EOI: Potential neurocognitive events^{e} | 0 (0.0) | 1 (1.0) |

^{a}Grade 3 events included: one event of neurogenic shock (verbatim: vasovagal shock) assessed as nonserious and unrelated to IP; one event of headache assessed as nonserious and unrelated to IP; one event of blood creatine phosphokinase increased due to intense physical activity and assessed as nonserious and unrelated to IP; and one event of cholelithiasis assessed as serious and unrelated to IP. No subject discontinued IP due to a grade 3 adverse event.
^{b}Subject developed abdominal pain within days of starting IP and was subsequently diagnosed with cholelithiasis (grade 3) on study day 35; event considered unrelated to IP and IP was continued.
^{c}Events of headache, oropharyngeal pain, influenza, upper respiratory tract infection (URTI), and influenza like illness were all nonserious and mostly grade 1 or 2 (one headache was grade 3), and none led to discontinuation of IP. These events are consistent with the known adverse drug reactions (ADRs) for Repatha described in the CDS or symptoms of those ADRs (i.e., influenza, influenza like illness, nasopharyngitis, and URTI).
^{d}Hypersensitivity including rash, urticaria, and angioedema are expected ADRs for Repatha; the reported events were all nonserious, grade 1 or 2, and consistent with those previously observed.
^{e}Nonserious, grade 1 event of disturbance in attention (verbatim: concentration impairment) on study day 30 and considered related to IP by the investigator; subject continued on IP and event outcome was not reported.

These results show inhibiting PCSK9 with evolocumab on a background of statin therapy in a pediatric HeFH patient population was safe and lowered LDL-C by 38.3%, or by 68.6 mg/dL, at 24 weeks.

### Example 2

This non-limiting example shows the results of subgroup analysis of the data in Example 1 based on baseline LDL-C quartiles. The outcomes were divided into subgroups defined by interquartile ranges of the baseline LDL-C for all subjects. The interquartile ranges were: Q1: LDL-C<154 mg/dL; Q2: 154≤LDL-C<173 mg/dL; Q3: 173≤LDL-C<208 mg/dL; and Q4: LDL-C≥208 mg/dL.

The results of the subgroup analysis by baseline LDL-C quartiles is shown in Tables 2.1 and 2.2.

TABLE 2.1
Mean of Week 22 and Week 24

| | Treatment | | | Treatment Difference | |
|---|---|---|---|---|---|
| Subgroup Level | Group | LSMean (SE) | | vs. placebo (SE) | p-value |
| Q1 (<154 mg/dL) | Placebo | 2.39 | (5.35) | −50.57 (4.81) | <0.001 |
| | evolocumab | −48.18 | (5.68) | | |
| Q2 (154 ≤ LDL-C < | Placebo | −0.35 | (5.46) | −49.92 (5.85) | <0.001 |
| 173 mg/dL) | evolocumab | −50.28 | (3.35) | | |
| Q3 (173 ≤ LDL-C < | Placebo | −14.90 | (8.45) | −43.64 (7.54) | <0.001 |
| 208 mg/dL) | evolocumab | −58.55 | (6.94) | | |
| Q4 (LDL-C ≥ | Placebo | −9.56 | (5.39) | −27.69 (6.65) | <0.001 |
| 208 mg/dL) | evolocumab | −37.25 | (3.89) | | |

Interaction p-value: 0.040

TABLE 2.2
Week 24

| | Treatment | | Treatment Difference | |
|---|---|---|---|---|
| Subgroup Level | Group | LSMean (SE) | vs. placebo (SE) | p-value |
| Q1 (<154 mg/dL) | Placebo | 0.49 (5.69) | −47.52 (5.40) | <0.001 |
| | evolocumab | −47.03 (5.80) | | |
| Q2 (154 ≤ LDL-C < | Placebo | −2.42 (6.81) | −39.66 (7.46) | <0.001 |
| 173 mg/dL) | evolocumab | −42.08 (4.02) | | |
| Q3 (173 ≤ LDL-C < | Placebo | −11.76 (8.65) | −45.25 (7.89) | <0.001 |
| 208 mg/dL) | evolocumab | −57.01 (7.08) | | |
| Q4 (LDL-C ≥ | Placebo | −11.75 (6.30) | −23.24 (7.78) | 0.005 |
| 208 mg/dL) | evolocumab | −34.99 (4.54) | | |

Interaction p-value: 0.085

These results show the percentage reduction in LDL-C by inhibiting PCSK9 with evolocumab on a background of statin therapy in a pediatric HeFH patient population can depend on the baseline LDL-C of the patient being treated. In some embodiments, patients with LDL-C at or above a threshold level show a blunted response to PCSK9 inhibition therapy. In some embodiments the threshold level is 208 mg/dL. In some embodiments, LDL-C in pediatric patients having baseline LDL-C within the top quartile of baseline LDL-C among the pediatric HeFH patient population is reduced to a lesser degree than in patients with baseline LDL-C within the lower three quartiles of baseline LDL-C in the pediatric HeFH patient population.

### Example 3

This non-limiting example shows treatment of a pediatric subject having HeFH with a PCSK9 inhibitor administered according to an enhanced dosage regimen.

A pediatric HeFH subject having a baseline LDL-C of 210 mg/dL is identified. The subject is administered 420 mg of evolocumab (REPATHA) subcutaneously every two weeks. The subject's LDL-C is thereby lowered by at least 30%.

### Example 4

This non-limiting example shows treatment of a pediatric subject having HeFH with a PCSK9 inhibitor administered according to an enhanced dosage regimen.

A pediatric HeFH subject having a baseline LDL-C of 210 mg/dL is identified. The patient is administered 490 mg of evolocumab (REPATHA) subcutaneously every four weeks. The patient's LDL-C is thereby lowered by at least 30%.

### Example 5

This non-limiting example shows treatment of a pediatric subject having HeFH with a PCSK9 inhibitor administered according to an enhanced dosage regimen.

A pediatric HeFH subject has a baseline LDL-C of 210 mg/dL. The subject's baseline LDL-C is greater than an upper quartile of baseline LDL-C values among a cohort of pediatric HeFH patients. The subject is administered a PCSK9 inhibitor at a dosing frequency twice the average dosing frequency for pediatric patients having a baseline LDL-C value that is less than the upper quartile. The subject's LDL-C is thereby lowered to a similar extent as the average reduction in LDL-C achieved by administering the PCSK9 inhibitor to pediatric HeFH patients having a baseline LDL-C value that is less than the upper quartile.

As shown in Examples 1 and 2 above, different subsets of pediatric patients exhibited different response to anti-PCSK9 therapy. It was observed herein that baseline LDL-C levels can be used to stratify the pediatric HeFH patients for responsiveness to the anti-PCSK9 therapy. These findings indicate better outcomes for anti-PCSK9 therapy can be achieved in pediatric patients having severe HeFH by adjusting the dosage regimen, e.g., by increasing the frequency of administration and/or increasing the dose. Further, smaller doses and/or lower frequencies of PCSK9 inhibitors can achieved greater percent reduction of LDL-C in pediatric patients having relatively low levels of baseline LDL-C.

Each reference cited herein is hereby incorporated by reference in its entirety for all that it teaches and for all purposes.

As used in this specification and the appended claims, the singular forms “a,” “an,” and “the” include plural references unless the context clearly dictates otherwise. Any reference to “or” herein is intended to encompass “and/or” unless otherwise stated.

As will be understood by one skilled in the art, for any and all purposes, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. As will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

The disclosed subject matter is not to be limited in scope by the specific embodiments described herein, which are intended as single illustrations of individual embodiments of the present disclosure, and functionally equivalent methods and components are contemplated. Indeed, various modifications of the present disclosure, in addition to those shown and described herein will become apparent to those skilled in the art from the foregoing description and accompanying drawings. Such modifications are intended to fall within the scope of the appended claims. In at least some of the previously described embodiments, one or more elements used in an embodiment can interchangeably be used in another embodiment. It will be appreciated by those skilled in the art that various other omissions, additions and modifications may be made to the methods and structures described above without departing from the scope of the claimed subject matter.

What is claimed is:

1. A method of lowering serum LDL cholesterol (LDL-C) in a pediatric subject, comprising:
identifying a pediatric subject having heterozygous familial hypercholesterolemia (HeFH), wherein the subject has a baseline LDL-C of about 200 mg/dL or greater; and
administering to the subject an anti-PCSK9 antibody at a dose from about 350 to about 500 mg, to thereby lower the subject's LDL-C.

2. The method of claim 1, wherein the baseline LDL-C is from about 200 mg/dL to about 550 mg/dL.

3. The method of claim 1 or 2, wherein the baseline LDL-C is 208 mg/dL or more.

4. The method of any one of the preceding claims, wherein the subject's LDL-C is lowered by at least 200%, at least 30%, at least 40%, about 30% to about 50%, about 20% to about 50%, about 20% to about 80%, about 30% to about 50%, or about 30% to about 80%.

5. The method of any one of the preceding claims, wherein the subject's LDL-C is lowered by at least 30%.

6. The method of any one of the preceding claims, wherein the subject's LDL-C is lowered by about 30% to about 80%.

7. The method of any one of the preceding claims, wherein the anti-PCSK9 antibody is administered every two weeks to every four weeks, every two weeks, or every four weeks.

8. The method of any one of claims 1-3, wherein the anti-PCSK9 antibody is administered every four weeks, and wherein the subject's LDL-C is lowered by at least 20%.

9. The method of any one of claims 1-3, wherein the anti-PCSK9 antibody is administered every two weeks, and wherein the subject's LDL-C is lowered by at least 30%.

10. A method of lowering serum LDL cholesterol (LDL-C) in a pediatric subject, comprising:
identifying a pediatric subject having heterozygous familial hypercholesterolemia (HeFH), wherein the subject has a baseline LDL-C of about 210 mg/dL or less; and
administering to the subject a PCSK9 antibody, at a dose of about 350 to about 500 mg, to thereby lower the subject's LDL-C, wherein the subject's LDL-C is lowered by at least 40%.

11. The method of claim 10, wherein the baseline LDL-C is less than 208 mg/dL.

12. The method of claim 10 or 11, wherein the subject's LDL-C is lowered by at least 40%, at least 50%, at least 60%, about 40% to about 60%, about 40% to about 80%, about 50% to about 60%, or about 50% to about 80%.

13. The method of any one of claims 10-12, wherein the subject's LDL-C is lowered by at least 45%.

14. The method of any one of claims 10-13, wherein the anti-PCSK9 antibody is administered every two weeks to every four weeks, every two weeks, or every four weeks.

15. The method of any one of claims 10-13, wherein the anti-PCSK9 antibody is administered every four weeks, and wherein the subject's LDL-C is lowered by at least 40%.

16. The method of any one of claims 10-13, wherein the anti-PCSK9 antibody is administered every two weeks, and wherein the subject's LDL-C is lowered by at least 50%.

17. The method of any one of the preceding claims, wherein the anti-PCSK9 antibody comprises:
a heavy chain variable region (VH) comprising:
a CDRH1, CDRH2, and a CDRH3 of a CDRH1, CDRH2, and a CDRH3, respectively, of a VH of evolocumab; and
an amino acid sequence at least 90% identical to the VH of evolocumab; and
a light chain variable region (VL) comprising:
a CDRL1, CDRL2, and a CDRL3 of a CDRL1, CDRL2, and a CDRL3, respectively, of a VL of evolocumab; and
an amino acid sequence at least 90% identical to the VL of evolocumab.

18. The method of any one of the preceding claims, wherein the anti-PCSK9 antibody is evolocumab.

19. The method of any one of the preceding claims, wherein the dose is about 420 mg.

20. The method of any one of claims 1-18, wherein the dose is about 490 mg.

21. The method of any one of the preceding claims, wherein the subject's LDL-C is lowered by at least week 20 of administration of the PCSK9 antibody.

22. A method of lowering serum LDL cholesterol (LDL-C) in a pediatric subject, comprising:
identifying a pediatric subject having heterozygous familial hypercholesterolemia (HeFH), wherein the subject has a baseline LDL-C at or above an upper quartile of baseline LDL-C values among a pediatric HeFH patient cohort; and
administering to the subject an enhanced dosage regimen of a PCSK9 inhibitor,
wherein the enhanced dosage regimen comprises an amount and/or dosing frequency that is each independently about 20% to about 500% greater than an average amount and/or average dosing frequency of the PCSK9 inhibitor for pediatric HeFH patients having a baseline LDL-C value that is less than the upper quartile, whereby the subject's LDL-C is lowered.

23. The method of claim 22, wherein the amount of the PCSK9 inhibitor is about 5% to about 100% greater than the average amount.

24. The method of claim 22 or 23, wherein the dosing frequency of the PCSK9 inhibitor is about 15% to about 400% greater than the average dosing frequency.

25. The method of any one of claims 22-24, wherein the average dosing frequency is a dosing frequency of the PCSK9 inhibitor for pediatric HeFH patients having a baseline LDL-C value that is less than a median of baseline LDL-C values among the cohort.

26. The method of any one of claims 22-25, wherein the average amount is an amount of the PCSK9 inhibitor for pediatric HeFH patients having a baseline LDL-C value that is less than a median of baseline LDL-C values among the cohort.

27. The method of any one of claims 22-26, wherein the subject's LDL-C is lowered by at least 30%.

28. The method of any one of claims 22-27, wherein the subject's LDL-C is lowered by from about 30% to about 80%.

29. The method of any one of claims 22-28, wherein the reduction in the subject's LDL-C is at least 70% of the average reduction in LDL-C achieved in pediatric HeFH patients having a baseline LDL-C value that is less than the upper quartile and receiving the PCSK9 inhibitor at the average frequency of administration.

30. The method of any one of claims 22-29, wherein the subject's LDL-C is lowered by at least week 20 of administration of the PCSK9 inhibitor.

31. A method of treating or preventing heterozygous familial hypercholesterolemia (HeFH) or symptoms thereof, comprising:
identifying a pediatric subject in need of treatment or prevention of HeFH or symptoms thereof, wherein the subject has a baseline LDL-C at or above an upper quartile of baseline LDL-C values among a pediatric HeFH patient cohort; and
administering to the subject an enhanced dosage regimen of a PCSK9 inhibitor, to thereby treat or prevent HeFH or symptoms thereof,
wherein the enhanced dosage regimen comprises administering the PCSK9 inhibitor at a mean dose that is about 20% to about 500% greater than a reference mean dose of the PCSK9 inhibitor for treating or preventing HeFH or symptoms thereof in pediatric HeFH patients having a baseline LDL-C value that is less than the upper quartile.

32. The method of claim 31, wherein the reference mean dose is a dose of the PCSK9 inhibitor for pediatric HeFH patients having a baseline LDL-C value that is less than a median of baseline LDL-C values among the cohort.

33. The method of claim 31 or 32, wherein the enhanced dosage regimen comprises an increase in a dosing frequency and/or an amount of the PCSK9 inhibitor administered to the subject.

34. The method of any one of claims 22-33, wherein the upper quartile is in a range of about 190 mg/dL to about 220 mg/dL.

35. The method of any one of claims 22-34, wherein the upper quartile is about 200 mg/dL.

36. The method of any one of claims 22-35, wherein the subject's baseline LDL-C is about 200 mg/dL or greater.

37. The method of any one of claims 22-36, wherein the baseline LDL-C is from about 200 mg/dL to about 550 mg/dL.

38. The method of any one of claims 22-37, wherein the baseline LDL-C is 208 mg/dL or greater.

39. The method of any one of claims 22-38, wherein the PCSK9 inhibitor is approved by a government regulatory agency for lowering serum LDL cholesterol levels in a human patient.

40. The method of any one of claims 22-39, wherein the average dosing frequency is a dosing frequency of the PCSK9 inhibitor for pediatric HeFH patients having a baseline LDL-C value that is less than a median of baseline LDL-C values among the cohort.

41. The method of any one of claims 22-40, wherein the PCSK9 inhibitor is an antibody, small-molecule inhibitor, or an inhibitory nucleic acid.

42. The method of claim 41, wherein the PCSK9 inhibitor is an anti-PCSK9 antibody, a siRNA or shRNA.

43. The method of claim 41, wherein the PCSK9 inhibitor comprises one or more of evolocumab, alirocumab, bococizumab, 1D05-IgG2, RG-7652, LGT209, REGN728, LY3015014, 1B20, inclisiran, ISIS 394814, ALN-PCS02, SX-PCK9, and BMS-962476.

44. The method of any one of claims 22-43, wherein the average dosing frequency is in a range from about once every 2 weeks to about once every 12 weeks.

45. The method of any one of claims 22-44, further comprising determining quartiles of the baseline LDL-C values of the cohort.

46. The method of any one of claims 22-45, wherein the cohort comprises at least 25 pediatric HeFH patients.

47. The method any one of claims 22-46, wherein the baseline LDL-C values among the cohort is at least 130 mg/dL.

48. The method of any one of the preceding claims, further comprising measuring the baseline LDL-C of the subject.

49. The method of any one of the preceding claims, wherein the identifying comprises diagnosing and/or genotyping the subject for HeFH.

50. The method of any one of the preceding claims, wherein the identifying comprises diagnosing and/or genotyping the patient for compound HeFH.

51. A method of lowering serum LDL-cholesterol (LDL-C) in a pediatric subject, the method comprising:
administering to a pediatric subject an enhanced dosage regimen of a PCSK9 inhibitor,
wherein the subject has heterozygous familial hypercholesterolemia (HeFH) or symptoms thereof,
wherein the enhanced dosage regimen of the PCSK9 inhibitor comprises an amount of the PCSK9 inhibitor that is about 20% to about 500% greater than a standard-of-care average amount for adults having HeFH, and/or
a dosing frequency of the PCSK9 inhibitor that is about 20% to about 500% greater than a standard-of-care average frequency for adults having HeFH, whereby the subject's LDL-C is lowered.

52. The method of claim 51, wherein the enhanced dosage regimen lowers LDL-C in the subject by at least 30%.

53. The method of claim 51 or 52, wherein the enhanced dosage regimen lowers LDL-C in the subject by 30%-80%.

54. The method of any one of claims 51-53, wherein the amount of the PCSK9 inhibitor is increased by about 5% to about 100% than the standard-of-care amount.

55. The method of any one of claims 51-54, wherein the dosing frequency of the PCSK9 inhibitor is increased by about 15% to about 400% than the standard-of-care dosing frequency.

56. The method of any one of claims 51-55, wherein the enhanced dosage regimen is continued until a therapeutically acceptable end point for HeFH is achieved.

57. The method of any one of claims 51-56, wherein the PCSK9 inhibitor is approved by government regulatory agency for lowering LDL-C in a human patient.

58. The method of any one of claims 51-57, wherein the standard-of-care dosing frequency is between once every 2 weeks to once every 12 weeks.

59. The method of any one of claims 51-58, wherein the PCSK9 inhibitor is an anti-PCSK9 antibody.

60. The method of claim 59, wherein the anti-PCSK9 antibody comprises:
a heavy chain variable region (VH) comprising:
a CDRH1, CDRH2, and a CDRH3 of a CDRH1, CDRH2, and a CDRH3, respectively, of a VH of evolocumab; and
an amino acid sequence at least 90% identical to the VH of evolocumab; and
a light chain variable region (VL) comprising:
a CDRL1, CDRL2, and a CDRL3 of a CDRL1, CDRL2, and a CDRL3, respectively, of a VL of evolocumab; and
an amino acid sequence at least 90% identical to the VL of evolocumab.

61. The method of claim 59 or 60, wherein the anti-PCSK9 antibody is evolocumab.

62. The method of any one of claims 59-61, wherein the standard-of-care amount is between 400 and 500 mg/dose.

63. The method of any one of claims 59-62, wherein the standard-of-care amount and/or frequency is about 420 mg/month.

64. The method of any one of claims 51-63, wherein the subject's LDL-C is lowered by at least week 20 of administration of the PCSK9 inhibitor.

65. A method of treating or preventing heterozygous familial hypercholesterolemia (HeFH) or symptoms thereof, comprising:
identifying a pediatric subject in need of treatment or prevention of HeFH or symptoms thereof; and
administering to the subject an enhanced dosage regimen of a PCSK9 inhibitor, to thereby treat or prevent HeFH or symptoms thereof,
wherein the enhanced dosage regimen comprises administering the PCSK9 inhibitor at an mean dose that is about 20% to about 500% greater than a standard-of-care mean dose of the PCSK9 inhibitor to treat or prevent HeFH or symptoms thereof in an adult patient.

66. The method of claim 65, wherein the enhanced dosage regimen comprises a higher dosing frequency of the PCSK9 inhibitor than a standard-of-care dosing frequency.

67. The method of claim 65 or 66, wherein the enhanced dosage regimen comprises a higher amount of the PCSK9 inhibitor than a standard-of-care amount.

68. The method of any one of claims 51-67, wherein the PCSK9 inhibitor is an antibody, small-molecule inhibitor, or an inhibitory nucleic acid.

69. The method of claim 68, wherein the PCSK9 inhibitor is an anti-PCSK9 antibody.

70. The method of claim 68, wherein the PCSK9 inhibitor is a siRNA or shRNA.

71. The method of claim 68, wherein the PCSK9 inhibitor comprises one or more of evolocumab, alirocumab, bococizumab, 1D05-IgG2, RG-7652, LGT209, inclisiran, ISIS 394814, SX-PCK9, and BMS-962476.

72. The method of any one of the preceding claims, further comprising administering one or more other LDL cholesterol-lowering therapy to the subject.

73. The method of claim 72, wherein the other LDL cholesterol-lowering therapy comprises a statin, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IB AT inhibitor, a squalene synthase inhibitor, or a monocyte chemoattractant protein (MCP)-I inhibitor.

74. A method of lowering serum LDL cholesterol (LDL-C) in a pediatric subject, comprising:
administering to a pediatric subject having HeFH, wherein the subject has a baseline LDL-C of about 200 mg/dL or greater:
a PCSK9 antibody at a dosing frequency of about once a month, and at an amount from about 400 mg to about 450 mg;
at least one statin; and
at least one other LDL cholesterol-lowering therapy that is different from the PCSK9 antibody and the at least one statin,
to thereby lower the subject's LDL-C by at least 30%.

75. The method of claim 74, wherein the anti-PCSK9 antibody comprises:
a heavy chain variable region (VH) comprising:
a CDRH1, CDRH2, and a CDRH3 of a CDRH1, CDRH2, and a CDRH3, respectively, of a VH of evolocumab; and
an amino acid sequence at least 90% identical to the VH of evolocumab; and
a light chain variable region (VL) comprising:
a CDRL1, CDRL2, and a CDRL3 of a CDRL1, CDRL2, and a CDRL3, respectively, of a VL of evolocumab; and
an amino acid sequence at least 90° %6 identical to the VL of evolocumab.

76. The method of claim 74 or 75, wherein the anti-PCSK9 antibody is evolocumab.

77. The method of any one of claims 74-76, wherein the amount is about 420 mg.

78. A method of treating or preventing heterozygous familial hypercholesterolemia (HeFH) or symptoms thereof, comprising:
administering to a pediatric subject having HeFH and a baseline serum LDL cholesterol (LDL-C) at or above an upper quartile of baseline LDL-C values among a pediatric HeFH patient cohort:
a PCSK9 inhibitor;
at least one statin; and
at least one other LDL cholesterol-lowering therapy that is different from the PCSK9 inhibitor and the at least one statin,
to thereby treat or prevent HeFH or symptoms thereof,
wherein the PCSK9 inhibitor is administered according to a dosage regimen of the PCSK9 inhibitor for pediatric HeFH patients having a baseline LDL-C value that is less than the upper quartile.

79. The method of claim 78, wherein the upper quartile is in a range of about 190 mg/dL to about 220 mg/dL.

80. The method of claim 78 or 79, wherein the baseline LDL-C is about 200 mg/dL or greater.

81. The method of any one of claims 78-80, wherein the PCSK9 inhibitor is administered according to a dosage regimen of the PCSK9 inhibitor for pediatric HeFH patients having a baseline LDL-C value that is less than a median of baseline LDL-C values among the cohort.

82. A method of treating or preventing heterozygous familial hypercholesterolemia (HeFH) or symptoms thereof, comprising:
administering to a pediatric subject having HeFH:
a PCSK9 inhibitor, wherein the PCSK9 inhibitor is administered according to a standard-of-care dosage regimen to treat or prevent HeFH or symptoms thereof in an adult patient;
at least one statin; and
at least one other LDL cholesterol-lowering therapy that is different from the PCSK9 inhibitor and the at least one statin,
to thereby treat or prevent HeFH or symptoms thereof.

83. The method of claim 82, wherein the at least one other LDL cholesterol-lowering therapy is administered according to an enhanced dosage regimen comprising an mean dose of the at least one other LDL cholesterol-lowering therapy that is about 20% to about 500% greater than a standard-of-care mean dose of the at least one other LDL cholesterol-lowering therapy to treat or prevent HeFH or symptoms thereof in a pediatric patient.

84. The method of claim 83, wherein the enhanced dosage regiment comprises an increase in a dosing frequency and/or an increase in an amount of the PCSK9 inhibitor.

85. The method of any one of claims 78-84, wherein the PCSK9 inhibitor is an antibody, small-molecule inhibitor, or an inhibitory nucleic acid.

86. The method of claim 85, wherein the PCSK9 inhibitor comprises one or more of evolocumab, alirocumab, bococizumab, 1D05-IgG2, RG-7652, LGT209, REGN728, LY3015014, 1B20, inclisiran, ISIS 394814, SX-PCK9, and BMS-962476.

87. The method of any one of claims 74-86, wherein the at least one other LDL cholesterol-lowering therapy comprises a second PCSK9 inhibitor.

88. The method of claim 87, wherein the second PCSK9 inhibitor is a small-molecule inhibitor, or an inhibitory nucleic acid.

89. The method of claim 88, wherein the second PCSK9 inhibitor comprises one or more of evolocumab, alirocumab, bococizumab, 1D05-IgG2, RG-7652, LGT209, REGN728, LY3015014, 1B20, inclisiran, ISIS 394814, SX-PCK9, and BMS-962476.

90. The method of any one of claims 74-89, wherein the at least one other LDL cholesterol-lowering therapy comprises a statin, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IB AT inhibitor, a squalene synthase inhibitor, or a monocyte chemoattractant protein (MCP)-I inhibitor.

91. The method of any one of the preceding claims or claims 99-102, wherein the age of the subject is 17 years old or younger.

92. The method of any one of the preceding claims or claims 99-102, wherein the age of the subject is between 10 and 17 years old.

93. The method of any one of the preceding claims or claims 99-102, wherein the subject has compound HeFH.

94. The method of any one of the preceding claims or claims 99-102, wherein the subject is receiving at least one other LDL cholesterol-lowering therapy.

95. The method of any one of the preceding claims or claims 99-102, wherein the PCSK9 inhibitor or anti-PCSK9 antibody is administered subcutaneously or intravenously.

96. A kit for treating a pediatric subject in need of treating or preventing heterozygous familial hypercholesterolemia (HeFH) or symptoms thereof, comprising:
a dosage form comprising a PCSK9 inhibitor in an amount sufficient to administer the PCSK9 inhibitor to a pediatric subject having HeFH an enhanced dosage regimen comprising administering to the subject the PCSK9 inhibitor at a dosing frequency that is at least 2 fold greater than an average dosing frequency of the PCSK9 inhibitor for pediatric HeFH patients having a baseline LDL-C value that is less than the upper quartile.

97. The kit of claim 96, wherein the average dosing frequency is a dosing frequency of the PCSK9 inhibitor for pediatric HeFH patients having a baseline LDL-C value that is less than a median of baseline LDL-C values among the cohort.

98. A kit for treating a pediatric subject in need of treating or preventing heterozygous familial hypercholesterolemia (HeFH) or symptoms thereof, comprising:
a dosage form comprising a PCSK9 inhibitor in an amount sufficient to administer the PCSK9 inhibitor to a pediatric subject an enhanced dosage regimen comprising administering to the subject the PCSK9 inhibitor at a dosage that is about 20% to about 500% greater than a standard-of-care dosage of the PCSK9 inhibitor to treat or prevent the cholesterol-related disorder in an adult HeFH patient.

99. A method of lowering serum LDL cholesterol (LDL-C), comprising;
administering to a subject a PCSK9 inhibitor,
wherein the subject has heterozygous familial hypercholesterolemia,
wherein the subject is a pediatric subject,
wherein the PCSK9 inhibitor is administered in an amount that is at least as effective as 420 mg of evolocumab,
wherein the PCSK9 inhibitor is administered at a frequency of every two weeks or more,
whereby the subject's LDL-C is reduced by more than 30%.

100. A method of lowering serum LDL cholesterol (LDL-C) in a subject, comprising:
identifying a pediatric subject having heterozygous familial hypercholesterolemia (HeFH), wherein the subject has a baseline LDL-C above an upper quartile of baseline LDL-C values among a pediatric HeFH patient cohort; and
administering to the subject an enhanced dosage regimen of a PCSK9 inhibitor,
wherein the enhanced dosage regimen comprises a dosing frequency and/or an amount that is from 20% to 500% greater than an average dosing frequency and/or average amount in a government regulatory agency-approved label for the PCSK9 inhibitor, whereby the subject's LDL-C is lowered by at least 30%.

101. The method of claim 100, wherein the enhanced dosage regimen comprises a dosing frequency that is at least 2 fold greater than the average dosing frequency.

102. The method of any one of claims 99-101, wherein the PCSK9 inhibitor comprises one or more of evolocumab, alirocumab, bococizumab, 1D05-IgG2, RG-7652, LGT209, REGN728, LY3015014, 1B20, inclisiran, ISIS 394814, ALN-PCS02, SX-PCK9, and BMS-962476.


SEQUENCE LISTING (114 sequences)

SEQ ID NO: 1 - protein, 662 aa, Homo sapiens
QEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQ

SEQ ID NO: 2 - protein, 662 aa, Homo sapiens
QEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQGQVTVACEEGWTLTGCSALPGTSHVLGAYAVDNTCVVRSRDVSTTGSTSEEAVTAVAICCRSRHLAQASQELQ

SEQ ID NO: 3 - DNA, 2079 bp, Homo sapiens
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccagtga

SEQ ID NO: 4 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQ

SEQ ID NO: 5 - DNA, 2076 bp, Homo sapiens
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgctccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccgcggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggggcaggtgaccgtggcctgcgaggagggctggaccctgactggctgcagcgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccag

SEQ ID NO: 6 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQGQVTVACEEGWTLTGCSALPGTSHVLGAYAVDNTCVVRSRDVSTTGSTSEEAVTAVAICCRSRHLAQASQELQ

SEQ ID NO: 7 - protein, 441 aa, Homo sapiens
EVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 215 aa, Homo sapiens
ESALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - protein, 447 aa, Artificial Sequence
  alirocumab Heavy chain
EVQLVESGGGLVQPGGSLRLSCAASGFTFNNYAMNWVRQAPGKGLDWVSTISGSGGTTNYADSVKGRFIISRDSSKHTLYLQMNSLRAEDTAVYYCAKDSNWGNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 220 aa, Artificial Sequence
  alirocumab Light chain
DIVMTQSPDSLAVSLGERATINCKSSQSVLYRSNNRNFLGWYQQKPGQPPNLLIYWASTRESGVPDRFSGSGSGTDFTLTISSLQAEDVAVYYCQQYYTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 444 aa, Artificial Sequence
  Bococizumab heavy chain
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 214 aa, Artificial Sequence
  Bococizumab light chain
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 114 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARYGMDVWGQGTTVTVSS

SEQ ID NO: 14 - protein, 115 aa, Homo sapiens
QIQLVQSGAEVKKPGASVKVSCKASGYPLTSYGISWVRQAPGQGLEWMGWISAYNGNTNYAQKVQGSVTMTTDTSTSTVYMELRSLRSDDTAVYYCARGYGMDVWGQGTTVTVSS

SEQ ID NO: 15 - protein, 115 aa, Homo sapiens
QVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSS

SEQ ID NO: 16 - protein, 115 aa, Homo sapiens; identical to SEQ ID NO: 15

SEQ ID NO: 17 - protein, 115 aa, Homo sapiens; identical to SEQ ID NO: 15

SEQ ID NO: 18 - protein, 115 aa, Homo sapiens
QVQLVQSGAEVKKPGASVKVSCKASGYTLTSYGISWVRQAPGQGLEWMGWISFYNGNTNYAQKVQGRVTMTTDTSTSTVYMELRSLRSDDTAVYYCARGYGMDVWGQGTTVTVSS

SEQ ID NO: 19 - protein, 115 aa, Homo sapiens
QVQLVQSGAEVKRPGASVKVSCKASGYTLTSYGISWVRQAPGQGLEWMGWISVYNGNTNYAQKVQGRVTMTTDTSTSTVYMELRSLSSDDTAVYYCARGYGMDVWGQGTTVTVSS

SEQ ID NO: 20 - protein, 115 aa, Homo sapiens
QIQLVQSGAEVKKPGASVKVSCKASGYTLTSYGISWVRQAPGQGLEWMGWISFYNGNTNYAQKVQGRVTMTTDTSTSTVYMELRSLRSDDTAVYFCARGYGMDVWGQGTTVTVSS

SEQ ID NO: 21 - protein, 115 aa, Homo sapiens
QVQLVQSGAEVKKPGASLKVSCKASGYSLTSYGISWVRQAPGQGLEWMGWISAYNGNTNYAQKVQGRVTMTTDTSTSTVYMEVRSLRSDDTAVYYCARGYGMDVWGQGTTVTVSS

SEQ ID NO: 22 - protein, 115 aa, Homo sapiens
QVQLVQSGAEVKKPGASVKVSCKASGYPLTSYGISWVRQAPGQGLEWMGWISAYNGNTNYAQKVQGRVTMTTDTSTSTVYMELRSLRSDDTAVYYCARGYGMDVWGQGTTVTVSS

SEQ ID NO: 23 - protein, 115 aa, Homo sapiens
QVQLVQSGAEVKKPGASVKVSCKASGYALTSYGISWVRQAPGQGLEWMGWISAYNGNTNYAQKVQGRVTMTTDTSTSTVYMELRSLRSDDTAVYYCARGYGMDVWGQGTTVTVSS

SEQ ID NO: 24 - protein, 115 aa, Homo sapiens
QVQLVQSGAEVKKPGASVKVSCKASGYSFTSYGISWVRQAPGQGLEWMGWVSAYNGNTNYAQKFQGRVTMTTDTSTSTAYMELRSLRSDDTAVYYCARGYVMDVWGQGTTVTVSS

SEQ ID NO: 25 - protein, 115 aa, Homo sapiens
QVQLVQSGAEVKKPGASVKVSCKASGYTFPSYGISWVRQAPGQGLEWMGWISAYNGNTNYAEKLQGRVTMTTDTSTSTAYMEVRSLRSDDTAVFYCARGYVMDVWGQGTTVTVSS

SEQ ID NO: 26 - protein, 113 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGYDYWGQGTLVTVSS

SEQ ID NO: 27 - protein, 115 aa, Homo sapiens
QVQLVQSGAEVKKPGASVKVSCKASGYTFTSYGISWVRQAPGQGLEWMGWISTYNGNTNYAQKVQGRVTMTTDTSTSTAYMELRSLRSDDTAVYYCARGYTRDYWGQGTLVTVSS

SEQ ID NO: 28 - protein, 116 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARNWGAFDVWGQGTMVTVSS

SEQ ID NO: 29 - protein, 119 aa, Homo sapiens
EVQLVESGGGLVQPGGSLRLSCAASGFTFSRYWMSWVRQAPGKGLEWVANIKHDGSEKYYVDSVKGRFTISRDNAKNSLYLQMNSLRAEDTAVYYCARESNWGFAFDVWGHGTMVTVSS

SEQ ID NO: 30 - protein, 116 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARNWGAFDIWGQGTMVTVSS

SEQ ID NO: 31 - protein, 119 aa, Homo sapiens
EVQLVESGGGLVQPGGSLRLSCVVSGFTFSSYWMSWVRQAPGKGLEWVANIKQDGSEKYYVDSVKGRFTISRDNAKNSLYLQMNSLRAEDTAVYYCARESNWGFAFDIWGQGTMVTVSS

SEQ ID NO: 32 - protein, 119 aa, Homo sapiens
EVQLVESGGGLVQPGGSLRLSCAASGLTFSNFWMSWVRQAPGKGLEWVANIKQDGSEKYYVDSVKGRFTISRDNAKNSLYLQMNSLRAEDTAVYSCTRESNWGFAFDIWGQGTMVTVSS

SEQ ID NO: 33 - protein, 123 aa, Homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDYDFWSGYYTAFDVWGQGTMVTVSS

SEQ ID NO: 34 - protein, 123 aa, Homo sapiens
EVQLVESGGGLVKPGGSLRLSCAASGFTFSSYSMNWVRQAPGKGLEWVSSISSSSSYISYADSVKGRFTISRDNAKNSLYLQMNSLRAEDTAVYFCARDYDFWSAYYDAFDVWGQGTMVTVSS

SEQ ID NO: 35 - protein, 112 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKFDYWGQGTLVTVSS

SEQ ID NO: 36 - protein, 117 aa, Homo sapiens
EVQLLESGGGLVQPGGSLRLSCAASGFTFSSYAMSWVRQAPGKGLEWVSTISGSGGRTYYADSVKGRFTISRDNSKNTLYLQMNSLRAEDTAVYYCAKEVGSPFDYWGQGTLVTVSS

SEQ ID NO: 37 - protein, 117 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLMVYADYWGQGTLVTVSS

SEQ ID NO: 38 - protein, 121 aa, Homo sapiens
EVQLLESGGGLVQPGGSLRLSCAASGFTFSSYAMNWVRQAPGKGLEWVSTISGSGDNTYYADSVKGRFTISRDNSKNTLYLQMNSLRAEDTAVYYCAKKFVLMVYAMLDYWGQGTLVTVSS

SEQ ID NO: 39 - protein, 121 aa, Homo sapiens
EVQLLESGGGLVQPGGSLRLSCAASGFTFSSYAMNWVRQAPGKGLEWVSTISGSGGNTYYADSVKGRFTISRDNSKNTLYLQMNSLRAEDTAVYYCAKKFVLMVYAMLDYWGQGTLVTVSS

SEQ ID NO: 40 - protein, 116 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARYYYGMDVWGQGTTVTVSS

SEQ ID NO: 41 - protein, 123 aa, Homo sapiens
QVQLVESGGGVVQPGRSLRLSCAASGFTFSSYGMHWVRQAPGKGLEWVAVIWYDGSDKYYADSVKGRFTISRDNSKNTLYLQMNSLRAEDTAVYYCARETGPLKLYYYGMDVWGQGTTVTVSS

SEQ ID NO: 42 - protein, 116 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARIAAGMDVWGQGTTVTVSS

SEQ ID NO: 43 - protein, 122 aa, Homo sapiens
QVHLVESGGGVVQPGRSLRLSCAASGFTFNSFGMHWVRQAPGKGLEWVALIWSDGSDKYYADSVKGRFTISRDNSKNTLYLQMNSLRAEDTAVYYCARAIAALYYYYGMDVWGQGTTVTVSS

SEQ ID NO: 44 - protein, 122 aa, Homo sapiens
QVQLVESGGGVVQPGRSLRLSCAASGFTFSSFGMHWVRQAPGKGLEWVALIWNDGSNKYYADSVKGRFTISRDNSKNTLYLQMNSLRAEDTAVYYCARAIAALYYYYGMDVWGHGTTVTVSS

SEQ ID NO: 45 - protein, 122 aa, Homo sapiens
QVHLVESGGGVVQPGRSLRLSCAASGFTFSSFGMHWVRQAPGKGLEWVALIWNDGSNKYYADSVKGRFTISRDNSKNTLYLQMNSLRAEDTAVYYCARAIAALYYYYGMDVWGHGTTVTVSS

SEQ ID NO: 46 - protein, 122 aa, Homo sapiens
QVQLVESGGGVVQPGRSLRLSCAASGFTFSSFGMHWVRQAPGKGLEWVALIWNDGSNKYYADSVKGRFTISRDNSKNTLYLQMNSLRAEDTAVYYCARAIAALYYYYGMDVWGQGTTVTVSS

SEQ ID NO: 47 - protein, 122 aa, Homo sapiens
QVHLVESGGGVVQPGRSLRLSCAASGFTFNSFGMHWVRQAPGKGLEWVALIWSDGSDEYYADSVKGRFTISRDNSKNTLYLQMNSLRAEDTAVYYCARAIAALYYYYGMDVWGQGTTVTVSS

SEQ ID NO: 48 - protein, 122 aa, Homo sapiens; identical to SEQ ID NO: 46

SEQ ID NO: 49 - protein, 122 aa, Homo sapiens
QVQLVESGGGVVQPGRSLRLSCAASGFTFSSYGMHWVRQAPGKGLEWVAIIWYDGSNKYYADSVKGRFTISRDNSKNTLYLQMNSLRAEDTAVYYCARRGGLAARPGGMDVWGQGTTVTVSS

SEQ ID NO: 50 - protein, 122 aa, Homo sapiens
QVQLVESGGGVVQPGRSLRLSCAASGFTFSSYGMHWVRQAPGKGLECVAIIWYDGSNKYYADSVKGRFTISRDNSKNTLYLQMNSLRAEDTAVYYCARRGGLAARPGGMDVWGQGTTVTVSS

SEQ ID NO: 51 - protein, 122 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARGIAVAYYYYGMDVWGQGTTVTVSS

SEQ ID NO: 52 - protein, 122 aa, Homo sapiens
QVQLVESGGGVVQPGRSLRLSCAASGFTFRSYGMHWVRQAPGKGLEWVALIWHDGSNTYYVDSVKGRFTISRDNSKNTLYLQMNSLRAEDTAVYYCARGIAVAYYYYGMDVWGQGTTVTVSS

SEQ ID NO: 53 - protein, 117 aa, Homo sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARYYYGMDVWGQGTTVTVSS

SEQ ID NO: 54 - protein, 122 aa, Homo sapiens
QVQLQESGPGLVKPSQTLSLTCTVSGGSISSSDYYWSWIRQHPGKGLEWIGYIYYSGSTYYNPSLKSRITISVDTSKNLFSLKLSSVTAADTAVYYCARGGVTTYYYAMDVWGQGTTVTVSS

SEQ ID NO: 55 - protein, 120 aa, Homo sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCAREDTAMVYFDYWGQGTLVTVSS

SEQ ID NO: 56 - protein, 121 aa, Homo sapiens
QVQLQESGPGLVKPSQTLSLTCTVSGGSISSGGYYWSWIRQHPGKGLEWIGYIYNSGSTYYNPSLKSRVTISVDTSKNQFSLKLSSVTAADTAVYYCAREDTAMVPYFDYWGQGTLVTVSS

SEQ ID NO: 57 - protein, 115 aa, Homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGQLVFDYWGQGTLVTVSS

SEQ ID NO: 58 - protein, 116 aa, Homo sapiens
QVQLQQWGAGLLKPSETLSLTCAVYGGSFSAYYWNWIRQPPGKGLEWIGEINHSGRTDYNPSLKSRVTISVDTSKKQFSLKLNSVTAADTAVYYCARGQLVPFDYWGQGTLVTVSS

SEQ ID NO: 59 - protein, 115 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARFDYWGQGTLVTVSS

SEQ ID NO: 60 - protein, 121 aa, Homo sapiens
QVQLQQSGPGLVKPSQTLSLTCAISGDSVSSNSAAWNWIRQSPSRGLEWLGRTYYRSKWYKNYSVSVKSRITINPDTSKNQFSLQLNSVTPGDTAVYYCARGGPTAAFDYWGQGTLVTVSS

SEQ ID NO: 61 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGPGTKVDIK

SEQ ID NO: 62 - protein, 112 aa, Homo sapiens
DIVMTQSPLSLSVTPGEPPSISCRSSQSLLHSNGYNFLNWYLQKPGQSPQLLIYLGSHRASGVPDRFSGSGSGTDFTLEISRVEAEDVGVYYCMQVLQTPFTFGPGTKVDIK

SEQ ID NO: 63 - protein, 107 aa, Homo sapiens [sequence omitted]

SEQ ID NO: 64 - protein, 107 aa, Homo sapiens
DIQMTQSPSSLSASVGDRVTITCRASQRISNYLSWYLQKPGIAPKLLIYAASSLQSGVPSRFSGSGSGTDFTLTISSLQSEDFATYYCQQSYSTPLIFGGGTKVEIK

SEQ ID NO: 65 - protein, 107 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGQGTRLEIK

SEQ ID NO: 66 - protein, 107 aa, Homo sapiens
DILMTQSPSSLSASVGDRVTITCRASQSISSYLNWYQQKPGKAPKVLIYAASSLQSGVPSRFSGSGSGTDFTLTINSLQPEDFATYYCQQSYSSPITFGQGTRLEIK

SEQ ID NO: 67 - protein, 107 aa, Homo sapiens
DIQMTQSPSSLSASVGDRVTITCRASQSISIYLNWYQQKPGKAPYLLIYAAASLQSGVPSRFSGSGSGTDFTLTISSLQPEDFATYYCQQSYSAPITFGQGTRLEIK

SEQ ID NO: 68 - protein, 111 aa, Homo sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGGGTKLTVL

SEQ ID NO: 69 - protein, 111 aa, Homo sapiens
QSVLTQPPSVSGAPGQRVTISCTGSSSNIGAGYDVHWYQQLPGTAPKLLISGNSNRPSGVPDRFSGSKSGTSASLAITGLQAEDEADYYCQSYDSSLSGSVFGGGTKLTVL

SEQ ID NO: 70 - protein, 111 aa, Homo sapiens
QSVLTQPPSVSGAPGQRVTISCTGSSSNIGAHYDVHWYQQVPGTAPKLLIYGNTYRPSGVPDRFSGSKSGTSASLAITGLQAEDEADYYCQSYDNSLSGVVFGGGTKLTVL

SEQ ID NO: 71 - protein, 108 aa, Homo sapiens
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSVFGGGTKLTVL

SEQ ID NO: 72 - protein, 109 aa, Homo sapiens
QSALTQPASVSGSPGQSITISCTGTSSDVGRYNSVSWYQHHPGKAPKVMIYEVSNRPSGVSTRFSGSKSGNTASLTISGLQAEDEADYYCSSYTSSSVVFGGGTKLTVL

SEQ ID NO: 73 - protein, 109 aa, Homo sapiens
QSALTQPASVSGSPGQSITISCTGTSSDVGGYNSVSWYQQHPGKPPKLMIYEVSNRPSGVSIRFSGSKSGNTASLTISGLQAEDEADYFCSSYTSTSMVFGGGTKLTVL

SEQ ID NO: 74 - protein, 109 aa, Homo sapiens; identical to SEQ ID NO: 73

SEQ ID NO: 75 - protein, 109 aa, Homo sapiens
QSALTQPASVSGSPGQSITISCTGTSSDVGGYNSVSWYQQHPGKPPKLMIYEVSNRPSGVSNRFSGSKSGNTASLTISGLQAEDEADYFCSSYTSTSMVFGGGTKLAVL

SEQ ID NO: 76 - protein, 109 aa, Homo sapiens
QSALTQPASVSGSPGQSITISCTGTSSDVGGYNSVSWYQQYPGKPPKLKIYEVSNRPSGVSNRFSGSKSGNTASLTISGLQAEDEADYFCSSYTSTSMVFGGGTKLTVL

SEQ ID NO: 77 - protein, 109 aa, Homo sapiens
QSALTQPASVSGSPGQSITISCTGTSSDVGGYNSVSWYQQHPGKPPKLMIYEVSNRPSGVSNRFSGSKSGNTASLTISGLQAEDEADYFCSSYTSTSMVFGGGTKLTVL

SEQ ID NO: 78 - protein, 109 aa, Homo sapiens; identical to SEQ ID NO: 77

SEQ ID NO: 79 - protein, 109 aa, Homo sapiens
QSALTQPASVSGSPGQSITISCTGTNSDVGGYNSVSWYQQHPGKPPKLMIYEVSNRPSGISNRFSGSKSGNTASLTISGLQAEDEADYFCSSYTSTSMVFGGGTKLTVL

SEQ ID NO: 80 - protein, 109 aa, Homo sapiens
QSALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVL

SEQ ID NO: 81 - protein, 109 aa, Homo sapiens; identical to SEQ ID NO: 80

SEQ ID NO: 82 - protein, 109 aa, Homo sapiens
QSALTQPASVSGSPGQSITISCTGTSSDVGAYNSVSWYQQHPGKAPKRMIYEVSNRPSGVSNRFSGSKSGNTASLTISGLQAEDEADYYCSSYTSTNMVFGGGTKLTVL

SEQ ID NO: 83 - protein, 108 aa, Homo sapiens
QSALTQPASVSGSPGQSITISCTGTSSDVGRYNSVSWYQHHPGKAPKVMIYEVSNRPSGVSTRFSGSKSGNTASLTISGLQAEDEADYYCSSYTSSSVVFGGGTKLTV

SEQ ID NO: 84 - protein, 108 aa, Homo sapiens
QSALTQPASVSGSPGQSITISCTGTSSDVGGYNSVSWYQQHPGKPPKLMIYEVSNRPSGVSIRFSGSKSGNTASLTISGLQAEDEADYFCSSYTSTSMVFGGGTKLTV

SEQ ID NO: 85 - protein, 107 aa, Homo sapiens
SALTQPASVSGSPGQSITISCTGTNSDVGGYNSVSWYQQHPGKPPKLMIYEVSNRPSGISNRFSGSKSGNTASLTISGLQAEDEADYFCSSYTSTSMVFGGGTKLTV

SEQ ID NO: 86 - protein, 108 aa, Homo sapiens; identical to SEQ ID NO: 71

SEQ ID NO: 87 - protein, 109 aa, Homo sapiens
QSALTQPASVSGSPGQSITISCTGTSSDVGGYNSVSWYQQHPGKAPKLMIYEVTNRPSGVSNRFSGSKSGNTASLTISGLQAEDEADYYCNSYTSTSMVFGGGTKLTVL

SEQ ID NO: 88 - protein, 108 aa, Homo sapiens
QSALTQPASVSGSPGQSITISCTGTSSDVGSYNLVSWYQQHPGKAPKLMIYEGSKRPSGVSNRFSGSKSGNTASLTISGLQAEDEADYYCCSYAGSSTFGGGTKLTVL

SEQ ID NO: 89 - protein, 110 aa, Homo sapiens
LSALTQPASVSGSPGQSITISCTGTSSDVGNYNLVSWYQQYSGKAPKLMIYEVSKRPSGVSNRFSGSKSGNTASLTISGLQAEDEADYYCCSYAGSSTLVFGGGTKLTVL

SEQ ID NO: 90 - protein, 108 aa, Homo sapiens
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNVFGGGTKLTVL

SEQ ID NO: 91 - protein, 109 aa, Homo sapiens
QSVLTQPPSASGTPGQRVTISCSGSSSNIGSKTVNWYQQVPGTAPKLLIYRNNQRPLGVPDRFSGSKSGTSASLAISGLQSEDEADYYCAAWDDSLNWVFGGGTKLTVL

SEQ ID NO: 92 - protein, 109 aa, Homo sapiens
QSVLTQPPSASGPPGQRVTISCSGSSSNIGSNTVNWYQQLPGTAPKLLIYSNNRRPSGVPDRFSGSKSGTSASLAISGLQSEDEADYYCAAWDDSLNWVFGGGTKLTVL

SEQ ID NO: 93 - protein, 110 aa, Homo sapiens
QSVLTQPPSASGTPGQRVTISCSGSSSNIGSNTVNWYQQLPGTAPKLLIYSNNQRPSGVPDRFSGSKSGTSASLAISGLQSEDEADYYCAVWDDSLNGWVFGGGTKLTVL

SEQ ID NO: 94 - protein, 109 aa, Homo sapiens
QSVLTQPPSASGTPGQRVTISCSGSSSNIGSKTVNWYQQFPGTAPKLLIYSNNRRPSGVPDRFSGSKSGTSASLAISGLQSEDEADYYCAAWDDSLNWVFGAGTKLTVL

SEQ ID NO: 95 - protein, 110 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAYVFGTGTKVTVL

SEQ ID NO: 96 - protein, 110 aa, Homo sapiens
QSVLTQPPSVSAAPGQKVTISCSGSSSNIGNNFVSWYQQLPGTAPKLLIYDYNKRPSGIPDRFSGSKSGTSATLGITGLQTGDEADYYCGTWDSSLSAYVFGTGTRVTVL

SEQ ID NO: 97 - protein, 110 aa, Homo sapiens
QSVLTQPPSVSAAPGQKVTISCSGSSSNIGNNFVSWYQQLPGTAPKLLIYDYNKRPSGIPDRFSGSKSGTSATLGITGLQTGDEADYYCGTWDSSLSGYVFGTGTRVTVL

SEQ ID NO: 98 - protein, 110 aa, Homo sapiens
QSVLTQPPSVSAAPGQKVTISCSGSSSNIGNNFVSWYQQFPGTAPKLLIYDYNKRPSGIPDRFSGSKSGTSATLGITGLQTGDEADYYCGTWDSSLSSYVFGTGTRVTVL

SEQ ID NO: 99 - protein, 110 aa, Homo sapiens; identical to SEQ ID NO: 97

SEQ ID NO: 100 - protein, 110 aa, Homo sapiens
QSVLTQPPTVSAAPGQKVTISCSGSSSNIGNNFVSWYQQLPGTAPKLLIYDYNKRPSGIPDRFSGSKSGTSATLGITGLQTGDEADYYCGTWDSSLSGYVFGTGTRVTVL

SEQ ID NO: 101 - protein, 110 aa, Homo sapiens
QSVLTQPPSVSAAPGQKVTISCSGSSSNIGNNFVSWYQQLPGTAPKLLIYDSNKRPSGIPDRFSGSKSGTSATLDITGLQTGDEADYYCGTWDSSLSAYVFGTGTKVTVL

SEQ ID NO: 102 - protein, 110 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVVFGGGTKLTVL

SEQ ID NO: 103 - protein, 110 aa, Homo sapiens
QSVLTQPPSVSAAPGQKVTISCSGSNSNIGNNYVSWYQQLPGTAPKLLIYDNNKRPSGIPDRFSGSNSGTSATLGITGLQTGDEADYYCGTWDSSLSAVVFGGGTKLTVL

SEQ ID NO: 104 - protein, 107 aa, Homo sapiens
SYELTQPPSVSVSPGQTASITCSGDKLGDKYACWYQQKPGQSPVLVIYQDSKRPSGIPERFSGSNSGNTATLTISGTQAMDEADYYCQAWDSSTAVVFGGGTKLTVL

SEQ ID NO: 105 - protein, 106 aa, Homo sapiens
SYELTQPPSVSVSPGQTARITCSGDKLGDKYACWYQQKPGQSPVLVIYQNTKWPLGIPERFSGSKSGNTVTLTISGTQAMDEADYYCQAWDSSTVVFGGGTKLTVL

SEQ ID NO: 106 - protein, 116 aa, Homo sapiens
QPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFVVVFGGGTKLTVL

SEQ ID NO: 107 - protein, 116 aa, Homo sapiens
QPVLTQPLFASASLGASVTLTCTLSSGYSSYEVDWYQQRPGKGPRFVMRVDTGGIVGSKGEGIPDRFSVLGSGLNRYLTIKNIQEEDESDYHCGADHGSGTNFVVVFGGGTKLTVL

SEQ ID NO: 108 - protein, 67 aa, Homo sapiens
VGTGGIVGSKGDGIPDRFSVLGSGLNRYLTIKNIQEEDESDYHCGADHGSGSNFVVVFGGGTKLTVL

SEQ ID NO: 109 - protein, 67 aa, Homo sapiens
VDTGGIVGSKGEGIPDRFSVLGSGLNRYLTIKNIQEEDESDYHCGADHGSGTNFVVVFGGGTKLTVL

SEQ ID NO: 110 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 111 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 112 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 113 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 121 aa, Homo sapiens
QVQLVESGGGVVQPGRSLRLSCAASGFTFSSFGMHWVRQAPGKGLEWVALIWNDGSNKYYADSVKGRFTISRDNSKNTLYLQMNSLRAEDTAVYYCARAIAALYYYYGMDVWGQGTTVTVS